(19) United States
(12) Patent Application Publication
(10) Pub. No.: US 2010/0136026 A1
(43) Pub. Date: Jun. 3, 2010

(54) Ship Inhibition to Direct Hematopoietic Stem Cells and Induce Extramedullary Hematopoiesis

(76) Inventors: William G. Kerr, Syracuse, NY (US); Amy Hazen, Houston, TX (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/670,360

(22) PCT Filed: Sep. 26, 2008
(86) PCT No.: PCT/US08/77940

§ 371 Date: Feb. 10, 2010

Related U.S. Application Data
(60) Provisional application No. 60/975,477, filed on Sep. 26, 2007.

(51) Int. Cl. A61K 39/395; A61K 31/7052; A61P 19/08

(52) U.S. Cl. 424/158.1; 514/44 A

(57) ABSTRACT

The present invention concerns inhibition of SHIP expression and/or function in mammalian subjects to direct HSC from the bone marrow to other sites in the body (such as the spleen and/or liver), where they then re-establish blood cell production. The method of the invention can be employed, for example, to rescue or enhance blood cell recovery during bone marrow impairment due to bone-seeking radioisotope exposure, chemotherapy, radiotherapy, infection, or genetic or congenital defects. Preferably, inhibition of SHIP expression and/or function is achieved by administering an effective amount of one or more SHIP inhibitors to the subject. In one embodiment, the subject is suffering from, or at risk of developing, bone marrow failure (such as an aplastic bone marrow failure syndrome). In another embodiment, the subject is suffering from, or at risk of, exposure to a bone-seeking radioisotope. In another embodiment, the subject is suffering from, or at risk of developing, fibrotic or damaged bone marrow due to chemotherapy and/or radiation therapy for cancer.

### CROSS-REFERENCE TO RELATED APPLICATION

The present application claims the benefit of U.S. Provisional Application Ser. No. 60/975,477, filed Sep. 26, 2007, which is hereby incorporated by reference herein in its entirety, including any figures, tables, nucleic acid sequences, amino acid sequences, and drawings.

### GOVERNMENT SUPPORT

The subject matter of this application has been supported by research grants from the National Institutes of Health under grant number R01 HL72523 and R21 DK071872. Accordingly, the government has certain rights in this invention.

### BACKGROUND OF THE INVENTION

The SH2 domain containing-5′ inositol phosphatase-1 (SHIP) has the potential to modulate multiple signaling pathways downstream of receptors that impact hematopoietic stem cell (HSC) biology. SHIP can catalyze the removal of the 5′ phosphate group from PI_{(3,4,5)}P3 (PIP3) (Damen et al., 1996). In this manner, SHIP regulates survival and proliferation of various hematopoietic cell types. The numbers of myeloid cells and osteoclasts are increased in SHIP-deficient mice due to enhanced activity of the phosphatidyl inositol 3-kinase (PI3K)/Akt signaling pathway that promotes their survival (Takeshita et al., 2002; Helgason et al., 1998; Liu et al., 1999).

Furthermore, the number of natural killer cells are increased in SHIP-deficient mice resulting in an enhancement of engraftment of allogeneic hematopoietic stem cell grafts (Wang et al., 2002). SHIP is also known to influence signaling pathways downstream of receptors for chemokines and cytokines involved in megakaryocytopoiesis and thrombopoiesis, such as Stromal-cell-derived-Factor 1 (SDF-1/CXCL-12) (Wang et al., 1998; Hamada et al., 1998; Hattori et al., 2001; Avecilla et al., 2004; Chemock et al., 2001), interleukin-3 (Liu et al., 1994), and thrombopoietin (TPO) (Lok et al., 1994; Drachman et al., 1997b). SHIP is phosphorylated after TPO binding to its receptor, c-mpl, leading to activation of PI3K that promotes cycling of megakaryocytes (MK) (Drachman et al., 1997b; Drachman et al., 1997a; Geddis et al., 2001). TPO influences MK development by controlling their proliferation, differentiation, survival and endoduplication (Kaushansky et al., 1994). Circulating platelets sequester free TPO, and thereby limit megakaryocytopoiesis during steady-state hematopoiesis (Kaushansky, 1998). Furthermore, SDF-1/CXCL12 induces transendothelial MK migration and platelet production in vitro (Wang et al., 1998; Harnada et al., 1998) and in vivo (Hattori et al., 2001). It has also been shown that it enhances human thrombocytopoiesis in xenotransplanted NOD/SCID mice (Perez et al., 2004). SHIP-deficient myeloid progenitors exhibit enhanced chemotaxis towards SDF-1/CXCL-12, indicating SHIP influences signaling downstream of CXCR-4 (Kim et al., 1999). In addition, SHIP has been shown to regulate PIP3 levels after thrombin or collagen activation of platelets (Giuriato et al., 1997; Giuriato et al., 2003).

Consistent with the hypothesis that SHIP plays an important role in HSC homeostasis and function, it has been observed that HSC proliferation and function are altered in SHIP^{-/-} mice (Desponts et al., 2006a). Despite expansion of the bone marrow stem cell compartment in SHP−/− mice, the cells are dysfunctional.

Bone marrow failure (BMF) represents a failure of the hematopoietic function of the bone marrow, which may occur as the result of bone marrow suppression (suppression of bone marrow activity, resulting in reduction in the number of platelets, red cells, and white cells, such as in aplastic anemia). BMF syndromes (BMFS) typically result from hematopoietic progenitor or hematopoietic stem cell failure within the bone marrow. Aplastic anemia (AA) is the most common type of BMFS. Other types of BMFS include myelodysplastic syndrome (MDS); paroxysmal nocturnal hemoglobinuria (PNH); pure red cell aplasia (PRCA); amegakaryocytic thrombocytopenic purpura (ATP); and large granular lymphocyte leukemia (LGL leukemia).

It would be advantageous to have available an effective clinical strategy for rescue or enhancement of blood cell recovery during bone marrow impairment due to bone-seeking radioisotope exposure, chemotherapy, radiotherapy, infection, or genetic or congenital defects, etc.

### BRIEF SUMMARY OF THE INVENTION

The present inventors have determined that, in SH2 domain containing-5′ inositol phosphatase-1 (SHIP)-deficient mice, hematopoietic stem cells (HSC) leave the bone marrow (BM) and enter the spleen and proceed to produce blood cells there (extramedullary hematopoiesis). The present invention concerns inhibition of SHIP expression and/or function in mammalian subjects to direct HSC from the bone marrow to other sites in the body (such as the spleen and/or liver), where they then re-establish blood cell production (partially or fully restoring hematopoiesis). Preferably, inhibition of SHIP expression and/or function is achieved by administering an effective amount of one or more SHIP inhibitors to the subject. The methods of the invention can be employed, for example, to rescue or enhance blood cell recovery during bone marrow impairment due to bone-seeking radioisotope exposure, chemotherapy, radiotherapy, infection, or genetic or congenital defects.

In one embodiment, the subject is suffering from, or at risk of developing, bone marrow failure (such as an aplastic bone marrow failure syndrome). In another embodiment, the subject is suffering from, or at risk of, exposure to a bone-seeking radioisotope. In another embodiment, the subject is suffering from, or at risk of developing, fibrotic or damaged bone marrow due to chemotherapy and/or radiation therapy for cancer. Optionally, SHIP inhibition can be used with one or more other therapeutic or preventative treatments.

### BRIEF DESCRIPTION OF THE DRAWINGS

FIGS. 1A and 1B show that SHIP-deficient splenocytes are enriched for cells with radioprotective capacity. Splenocytes from SHIP−/− (red step functions) or WT (black step functions) littermates were transplanted (i.v. route) into lethally irradiated (1100Rads) Ly5 congenic recipients. Survival was monitored for 30 days. FIG. 1A shows transplant of 500K splenocytes from SHIP−/− and WT donors into Ly5 congenic hosts (25 mice per transplant group). FIG. 1B shows transplant of 250K splenocytes from SHIP−/− and WT donors into Ly5 congenic hosts (9 mice per transplant group). Notobene: In our hands and others 1100 Rads of radiation from a 137Cs source is uniformly lethal by 14 days post-transplant. The statistical significance of the survival differences between mice transplanted with SHIP−/− and WT cells was determined by the Kaplan Meier log-rank test (p<0.05).

FIGS. 2A-2D show that SHIP-deficient splenocytes contain cells with significant blood cell repopulating capacity. Splenocytes from SHIP−/− or WT littermates were transplanted (i.v. route) into lethally irradiated (1100Rads) Ly5 congenic recipients. At 4 months post-transplant the blood, bone marrow (BM) and spleens of the surviving mice were analyzed by multi-parameter FACS analysis to determine whether SHIP−/− splenocytes contained HSC capable of both global and lineage-specific blood cell reconstitution. This analysis showed that SHIP−/− splenocytes are capable of significant global reconstitution (FIG. 2A), B lymphoid reconstitution (FIG. 2B), T lymphoid reconstitution (FIG. 2C) and myeloid reconstitution (FIG. 2D). In no instance was reconstitution of any cell lineage in any tissue significantly less than that from the residual HSC activity present in mice transplanted with WT splenocytes (p>0.05). Together with the results from FIGS. 1A and 1B, these findings indicate SHIP−/− splenocytes contain significant radioprotective and long-term repopulating activity—two properties of HSC.

FIGS. 3A-3C show that spleens of SHIP-deficient mice contain a cell population capable of renewing the HSC compartment following transplant to lethally irradiated hosts. Splenocytes from SHIP−/− or WT littermates were transplanted (i.v. route) into lethally irradiated (1100Rads) Ly5 congenic recipients. Four months post-transplant the spleens of mice transplanted mice with either SHIP−/− splenocytes (FIG. 3A) or WT splenocytes (FIG. 3B) were analyzed by 6-color flow cytometry to identify and quantitate donor HSC of the LSKCD48− phenotype (Kiel et al, 2005). (FIGS. 3A, 3B) CD48 vs SSC contour plots with CD48− cells indicated by the blue rectangular gate in 6 mice that received SHIP-1− splenocyte transplants (FIG. 3A) and 3 mice that received WT splenocyte transplants (FIG. 3B). Cells were previously gated for the Lin−Sca1+LcKit+ (LSK) phenotype before displaying the CD48 vs. SSC contour plots. (FIG. 3C) Significant numbers of LSKCD48− HSC of the donor Ly5 allotype were detected in all mice receiving SHIP−/− splenocyte transplants. In fact, there were significantly greater numbers of donor LSKCD48− HSC detected in the spleens of mice receiving SHIP−/− splenocytes as compared to mice that survived and received WT splenocytes [p<0.05]. These findings demonstrate that spleens in SHIP-deficient mice contain an HSC population capable of renewing the HSC compartment. This data and our previous findings demonstrate that spleens of SHIP-deficient mice contain a cell capable of radioprotection (FIGS. 1A and 1B), long-term multi-lineage repopulation (FIGS. 2A-2D) and renewal of the HSC compartment (FIGS. 3A-3C). Thus, all the major hallmarks of the long-term repopulating HSC (LT-HSC) are present in the spleens of SHIP-deficient mice and, moreover, that LT-HSC activity is significantly increased in spleens of SHIP-deficient mice. We conclude then that SHIP-deficiency shifts significant LT-HSC activity to the spleen. Thus, induction of SHIP-deficiency could be beneficial for treatment in situations where the bone marrow environment is no longer able to adequately support the LT-HSC and its blood-forming capacity.

FIGS. 4A-4E show that cytokine serum levels are increased in SHIP^{-/-} mice, which is evidence for a disrupted bone marrow microenvironment. Represented are (FIG. 4A) matrix metallopeptidase 9 (MMP-9), (FIG. 4B) Thrombopoietin (TPO), (FIG. 4C) stromal cell-derived factor 1 (SDF-1), (FIG. 4D) Granulocyte Colony Stimulating Factor (G-CSF) and (FIG. 4E) Interleukin 6 (IL-6) levels measured by ELISA in serum of SHIP−/− (black) and WT (gray) mice (n=3). Blood was obtained by sub-mandibular bleed, collected in a regular Eppendorf tube and left at RT for 4 hours to allow coagulation. The blood was then stored at 4 C overnight. The following day, blood clots were removed using a wooden stick and the remaining blood was centrifuged at 4000 RPM for 10 minutes at 4 C. The serum was then isolated by taking the supernatant and ELISA assays were performed in house according to standard protocols or the supernatant was sent for analysis at a custom based service at Charles Rivers Laboratories Inc., where ELISA for multiple cytokines and growth factors was performed. These findings demonstrate that the bone marrow HSC of SHIP-deficient mice reside in a disrupted microenvironment which leads to an increase in mobilized or circulating HSC. The disrupted microenvironment directs normally quiescent bone marrow resident HSC to circulate through the peripheral blood and spleen resulting in extra-medullary hematopoiesis. As demonstrated in previous figures, these spleen resident HSC in SHIP-deficient mice are capable of radioprotection (FIGS. 1A and 1B), long-term multi-lineage repopulation (FIGS. 2A-2D) and renewal of the HSC compartment (FIGS. 3A-3C). Thus, induction of SHIP-deficiency could be beneficial for treatment in situations where the bone marrow environment is no longer able to adequately support the LTHSC and its blood-forming capacity. SHIP-deficiency can be achieved using inhibitors of SHIP enzymatic function (e.g., SHIP expression) such as small molecules, polypeptides, peptidomimetics, and nucleic acid molecules (e.g., interfering RNA molecules, antisense, ribozymes).

FIGS. 5A-5G show that in situ deletion of SHIP does not compromise the capacity of HSC to mediate long-term multi-lineage repopulation. (FIG. 5A) Dual color contour plots for CD45.1 vs. CD45.2 staining that illustrate equal repopulation in PB at 60 days post-transplant prior to polyI:C treatment in two representative MxCreSHIP^{flox/flox}(CD45.2):WT-Ly5.1(CD45.1) BM chimeras. (FIG. 5B) Percentage of donor repopulation in the PB by MxCreSHIP^{flox/flox}(CD45.2) and WT-Ly5.1(CD45.1) HSC in MxCreSHIP^{flox/flox}(CD45.2):WT-Ly5.1(CD45.1) BM chimeras at 60 days post-transplant prior to polyI:C treatment. (FIG. 5C) Representative SHIP Western blot from CD45.1⁺CD45.2⁻ or CD45.2⁺CD45.1⁻ sorted cells isolated from splenocytes from MxCreSHIP^{flox/flox}(CD45.2):WT-Ly5.1(CD45.1) BM chimeras 5 months after polyI:C treatment. (FIG. 5D) Percentage of global repopulation in the PB by MxCreSHIP^{flox/flox}(CD45.2) and WT-Ly5.1(CD45.1) HSC in MxCreSHIP^{flox/flox}(CD45.2):WT-Ly5.1(CD45.1) BM chimeras at the indicated times following polyI:C treatment (n≧0.5). (FIG. 5E) Competitive Repopulating Unit (CRU) activity by MxCreSHIP^{flox/flox}(CD45.2) and WT-Ly5.1(CD45.1) HSC in the MxCreSHIP^{flox/flox}(CD45.2):WT-Ly5.1(CD45.1) BM chimeras 5 months after polyI:C treatment (n≧5). (FIG. 5F) Dual color contour plots for CD45.1 vs. CD45.2 staining that illustrate multi-lineage repopulation in PB 20 weeks after polyI:C treatment in a representative MxCreSHIP^{flox/flox}(CD45.2):WT-Ly5.1(CD45.1) BM chimera. (FIG. 5G) Percentage repopulation of the indicated lymphoid and myeloid cell lineages by MxCreSHIP^{flox/flox}(CD45.2) and WT-Ly5.1(CD45.1) HSC in MxCreSHIP^{flox/flox}(CD45.2):WT-Ly5.1(CD45.1) BM chimeras 5 months after polyI:C treatment (n≧5). Significance was established using the unpaired Student's T test (*p<0.05). Errors shown represent the SEM. [Black bars, cells derived from MxCreSHIP^{flox/flox} BM; Grey bars, cells derived from WT-Ly5.1 BM]

FIGS. 6A-6C show that in situ deletion of SHIP does not compromise homing and secondary repopulating capacity of HSC. (FIG. 6A) Flow cytometric quantitation of the relative contribution to KLSCD48 HSC by MxCreSHIP^{flox/flox}(CD45.2) or WT-Ly5.1(CD45.1) cells in the BM of MxCreSHIP^{flox/flox}(CD45.2):WT-Ly5.1(CD45.1) chimeras 5 months after polyI:C treatment (n≧3). (FIG. 6B) Flow cytometric quantitation of the relative contribution to KLSCD48 HSC by MxCreSHIP^{flox/flox}(CD45.2) cells in the BM of MxCreSHIP^{flox/flox}(CD45.2):WT-Ly5.1(CD45.1) chimeras vs. SHIP^{flox/flox}(CD45.2) cells in SHIP^{flox/flox}(CD45.2):WT-Ly5.1(CD45.1) control chimeras. Both sets of chimeras were analyzed 5 months after polyI:C treatment (n≧3). (FIG. 6C) Percentage global repopulation of PB at the indicated times by MxCreSHIP^{flox/flox}(CD45.2) and WT-Ly5.1(CD45.1) HSC after serial transplantation of 1×10⁶ WBM cells from MxCreSHIP^{flox/flox}(CD45.2):WT-Ly5.1(CD45.1) chimeras 5 months after polyI:C treatment (n≧3). Significance was established using the unpaired Student's T test. Errors shown represent the SEM. [Black bars, cells derived from MxCreSHIP^{flox/flox} BM (Cre+); Grey bars, cells derived from WT-Ly5.1 BM (WT) or SHIP^{flox/flox}(Cre−)]

FIGS. 7A-7F show that systemic induction of SHIP-deficiency compromises the capacity of HSC to mediate long-term multi-lineage repopulation. (FIG. 7A) Flow cytometric quantitation of KLSCD48 HSC in the BM of MxCreSHIP^{flox/flox}(CD45.2) mice and SHIP^{flox/flox}(CD45.2) controls 21 days after polyI:C treatment (n≧3) (FIG. 7B) Percentage of global repopulation in PB at the indicated times post-transplant by WBM cells from SHIP-ablated MxCreSHIP^{flox/flox}(CD45.2) donors and WT-Ly5.1(CD45.1) control donors (n≧9). (FIG. 7C) Competitive Repopulation Unit (CRU) activity from SHIP-ablated MxCreSHIP^{flox/flox}(CD45.2) donors and WT-Ly5.1(CD45.1) control donors (n≧9). (FIG. 7D) Representative dual color contour plots that illustrate the level of PB reconstitution at 24 weeks post-transplant from SHIP-ablated MxCreSHIP^{flox/flox}(CD45.2) BM donors and WT-Ly5.1(CD45.1) BM competitor donors. (FIG. 7E) Percentage PB repopulation for the indicated lymphoid or myeloid lineage by WBM cells in a competitive transplant of SHIP-ablated MxCreSHIP^{flox/flox}(CD45.2) and WT-Ly5.1(CD45.1) BM competitor cells (n≧9). (FIG. 7F) Percentage BM repopulation for the indicated myeloid (Mach, granulocytic (Gr1), megakaryocytic (CD41) or erythroid (Ter119) lineage by WBM cells in a competitive transplant of SHIP-ablated MxCreSHIP^{flox/flox}(CD45.2) and WT-Ly5.1(CD45.1) BM competitor cells (n≧9). Significance was established using the unpaired Student's T test (*p<0.05; **p<0.005; ***p<0.0001). Errors shown represent the SEM. [Black bars, cells derived from MxCreSHIP^{flox/flox} BM; Grey bars, cells derived from SHIP^{flox/flox} (FIG. 7A) or WT-Ly5.1 BM (FIGS. 7B, 7C, 7E, 7F)]

FIGS. 8A-8D show that in situ deletion of SHIP does not alter CXCR4 surface expression, however, systemic induction of SHIP-deficiency significantly alters CXCR4 surface expression. (FIG. 8A) A representative histogram of CXCR4 surface expression on KLSCD48 HSC cells derived from MxCreSHIP^{flox/flox}(CD45.2) or WT-Ly5.1(CD45.1) donors in the BM of MxCreSHIP^{flox/flox}(CD45.2):WT-Ly5.1(CD45.1) chimeras 5 months after polyI:C treatment. (FIG. 8B) Median Fluorescence Intensity (MFI) of CXCR4 staining on KLSCD48 HSC cells derived from MxCreSHIP^{flox/flox}(CD45.2) or WT-Ly5.1(CD45.1) donors in the BM of MxCreSHIP^{flox/flox}(CD45.2):WT-Ly5.1(CD45.1) chimeras 5 months after polyI:C treatment. (FIG. 8C) Representative histograms of CXCR4 surface expression on KLSCD48 HSC in the BM of MxCreSHIP^{flox/flox}(CD45.2) mice or SHIP^{flox/flox}(CD45.2) controls 21 days after polyI:C treatment. (FIG. 8D) Median Fluorescence Intensity (MFI) of CXCR4 staining on KLSCD48 HSC in the BM of MxCreSHIP^{flox/flox}(CD45.2) or SHIP^{flox/flox}(CD45.2) controls 21 days after polyI:C treatment. Significance was established using the unpaired Student's T test (*p<0.05). [Black boxes, cells derived from MxCreSHIP^{flox/flox} BM (Cre+); Grey boxes, cells derived from SHIP^{flox/flox} (Cre−) or WT-Ly5.1 BM (WT)]

FIGS. 9A-9D show that SHIP-deficiency alters production of HSC mobilization and homing factors. (FIG. 9A) Scatter plots indicating the mean and range for the concentration of MMP-9 in the blood plasma of SHIP^{-/-} and WT littennates. (FIG. 9B) Scatter plots indicating the mean and range for the concentration of VCAM-1 in the blood plasma of SHIP^{-/-} and WT littermates. (FIG. 9C) Scatter plots indicating the mean and range for the concentration of SDF-1/CXCL12 in the blood plasma of SHIP^{-/-} and WT littermates. (FIG. 9D) Scatter plots indicating the mean and range for the concentration of SDF-1/CXCL12 in the BM plasma of SHIP^{-/-} and WT littermates. The data is pooled from multiple assays derived from a minimum of 7 different samples for each genotype. Significance was established using the unpaired Student's T test (*p<0.05; **p<0.005; ***p<0.0001). [Black boxes, SHIP^{-/-} samples; Grey boxes, WT samples].

FIGS. 10A-10H show that SHIP-deficiency alters the BM microenvironment. (FIG. 10A) Representative SHIP Western blot from stromal cells cultured from the BM of SHIP^{-/-} and WT littermates. (FIG. 10B) SHIP Western blot from osteoblast cells cultured from the BM of SHIP^{-/-} and WT littermates. (FIG. 10C) pTyr Western blot on a SHIP immunoprecipitation of stromal cells cultured from the BM of SHIP^{-/-} and WT littermates. (FIG. 10D) Scatter plots indicating the mean and range for the concentration of SDF-1/CXCL12 in the cell culture supernatant of SHIP^{-/-} and WT BM stromal cells. Results are representative of independent experiments on stromal cultures prepared from three independent pairs of SHIP^{-/-} and WT mice. (FIG. 10E) Representative SHIP Western blot from CD45⁻Lin⁻ microenvironment cells sorted from the BM of SHIP^{-/-} and WT littermates. (FIG. 10F) SHIP Western blot from CD45⁻Lin⁻ microenvironment cells sorted from the BM of MxCreSHIP^{flox/flox} and SHIP^{flox/flox} littermates. (FIG. 10G) LEFT: Dual color contour plots demonstrating control samples used to determine the gating threshold of CD45⁻Lin⁻ microenvironment cells and RIGHT: dual color contour plots illustrating the CD45⁻Lin⁻ microenvironment cells present in the BM of polyI:C treated MxCreSHIP^{flox/flox} and SHIP^{flox/flox} littermates. (FIG. 10H) Scatter plots indicating the mean and range of CD45⁻Lin⁻ microenvironment cells present in the BM of SHIP^{-/-} and WT (Germline) or MxCreSHIP^{flox/flox} and SHIP^{flox/flox} (MxCre) littermates as indicated. Each lane on the Western blots (FIGS. 10A, 10B, 10C, 10E, 10F) represents a culture lysate derived from an independent mouse. Significance was established using the unpaired Student's T test (*p<0.05; **p<0.005; ***p<0.0001). [Black boxes, SHIP^{-/-} samples (FIGS. 10D, 10H) or MxCreSHIP^{flox/flox} samples (FIG. 10H); Grey boxes, WT samples (FIGS. 10D, 10H) or SHIP^{flox/flox} samples (FIG. 10H)].

FIGS. 11A-11B depict schematic illustration of transplantation models. (FIG. 11A) In Situ SHIP deletion BM transplants: MxCreSHIP^{flox/flox}(CD45.2⁺) 5×10⁵ WBM cells were co-transplanted (i.v.) with 5×10⁵ WBM competitor cells from WT-Ly5.1(CD45.1) mice into lethally irradiated CD45.1⁺45.2⁻ recipients. Prior to transplant the recipients received a split dose of 11-Gy. After 60 days PB was used to monitor donor engraftment. Subsequently recipient mice were treated with three poly I:C injections to delete SHIP in all cells derived from the MxCreSHIP^{flox/flox} graft. (FIG. 11B) Systemic SHIP deletion BM transplants: MxCreSHIP^{flox/flox}(CD45.2⁺) mice were pre-treated with three poly I:C injections to systemically delete SHIP. After 21 days SHIP deletion was confirmed by Western blot analysis of PBMC. Once deletion was confirmed, 5×10⁵ WBM cells from MxCreSHIP^{flox/flox}(CD45.2⁺) mice were co-transplanted (i.v.) with 5×10⁵ WBM competitor cells from WT-Ly5.1(CD45.1⁺) mice into lethally irradiated CD45.1⁺45.2⁺ recipients. The recipients received a split dose of 11-Gy at least 2-hours before transplantation.

FIG. 12 shows that SHIP^{-/-} cultures exhibit elongated and non-randomly oriented protrusions and organized growth along axes. Representative OB cells propagated from SHIP^{-/-} and WT femurs after 11, 13, and 23 days in culture. All images were generated using a Nikon TE2000 inverted microscope with a 10×0.25NA PlanFluor objective lens and a Retiga 1300 12 bit CCD QImaging camera. The images were acquired using IP Lab v3.6 software (Scanalytics of BD Biosciences).

FIG. 13 depicts homologous morphology between SHIP^{-/-} and WT stromal cultures. Representative stromal cells propagated from SHIP^{-/-} and WT femurs after 11 and 28 days in culture. All images were generated using a Nikon TE2000 inverted microscope with a 10×0.25NA PlanFluor objective lens and a Retiga 1300 12 bit CCD QImaging camera. The images were acquired using IP Lab v3.6 software (Scanalytics of BD Biosciences).

### BRIEF DESCRIPTION OF THE SEQUENCES

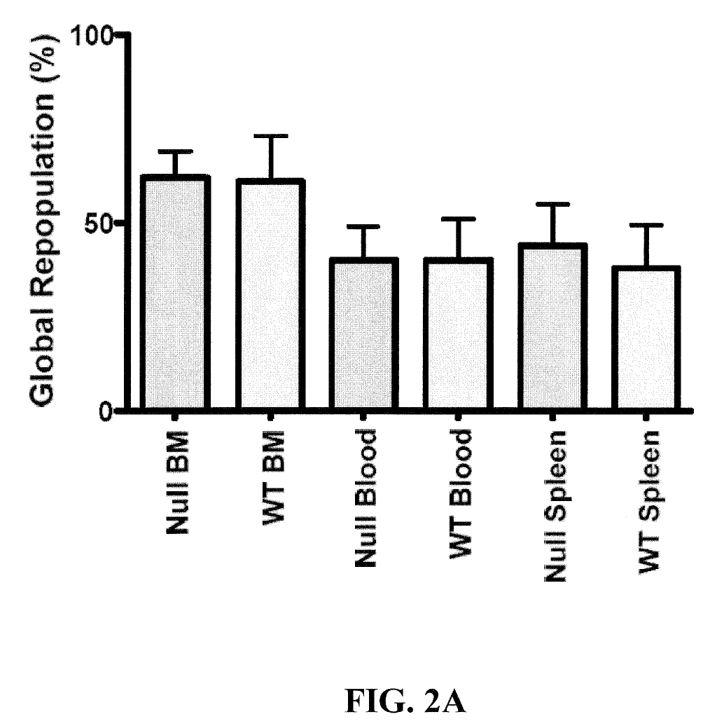

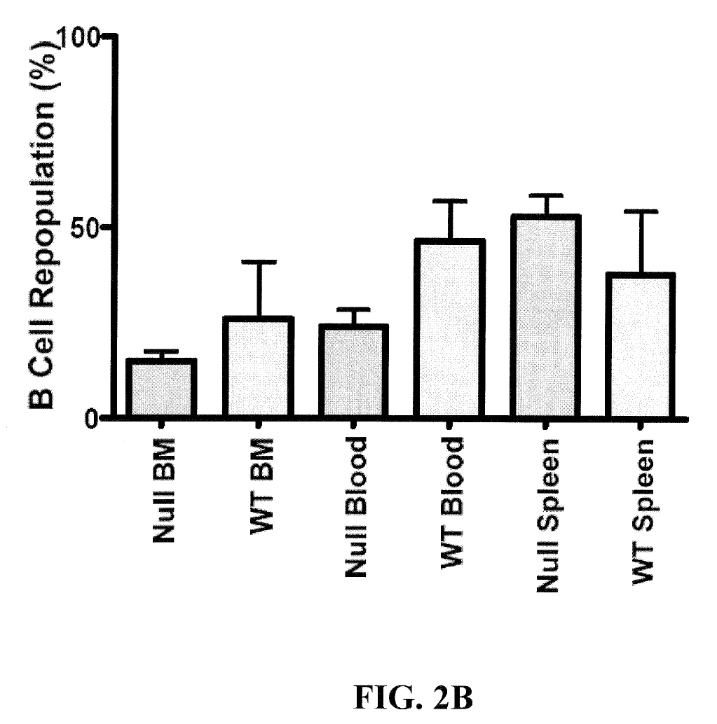

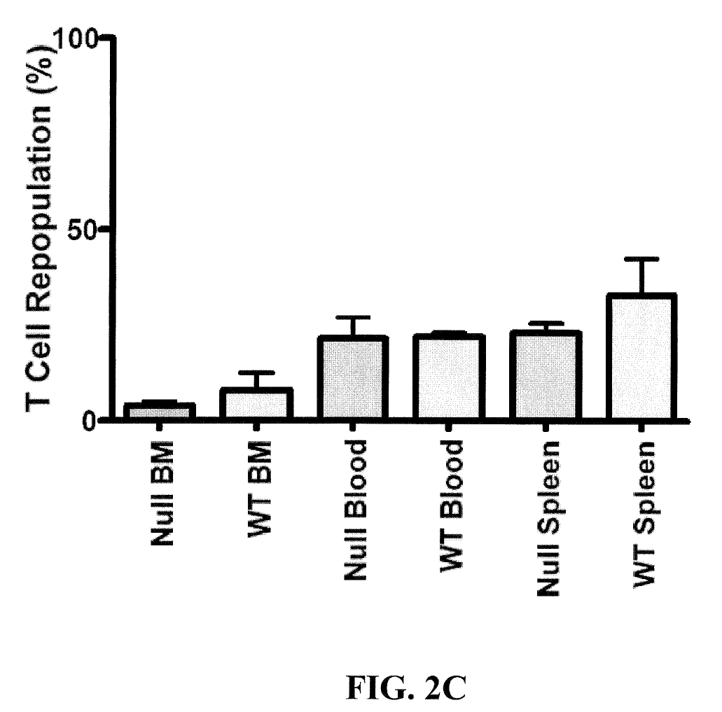

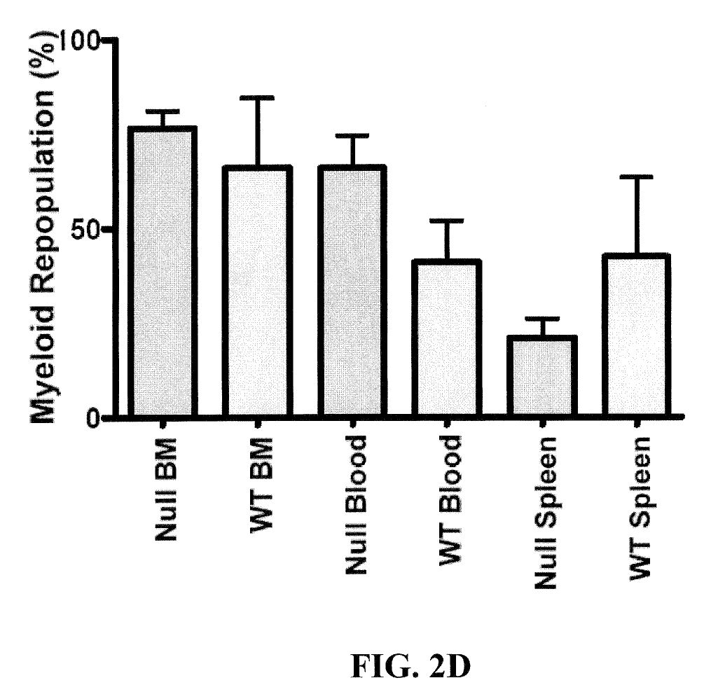

SEQ ID NO:1 is the human SHIP cDNA sequence (GenBank accession nos. NM_{—}005541 and NM_{—}001017915) (FIG. 2).

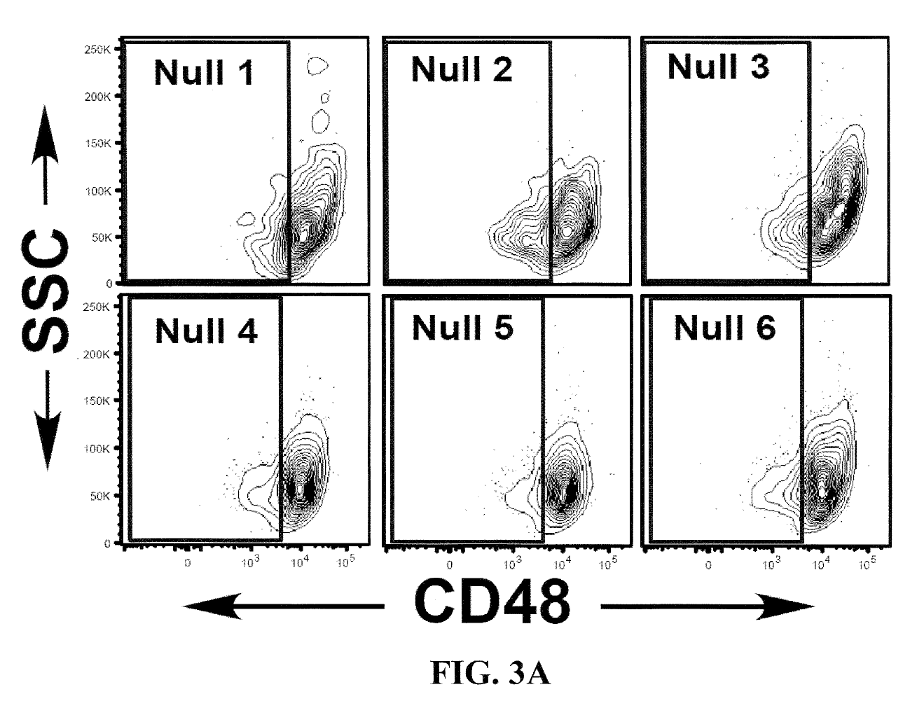

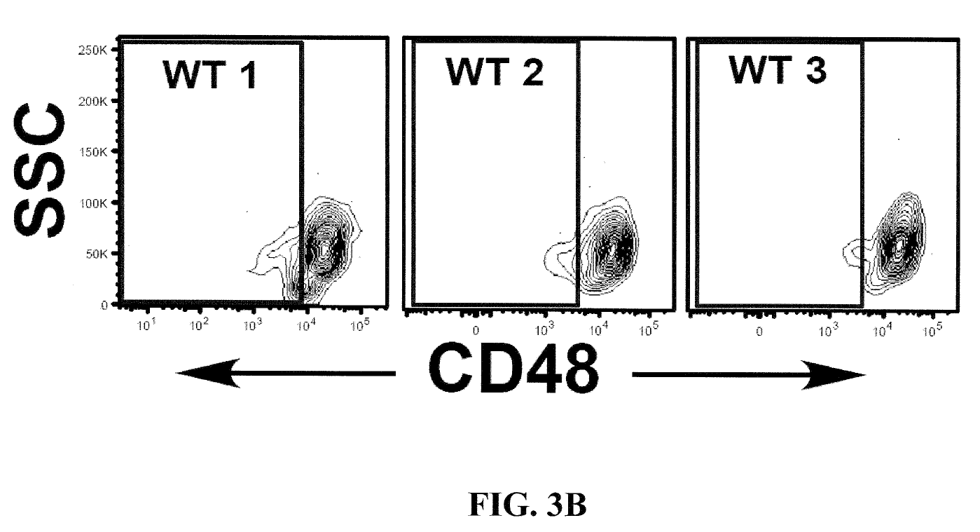

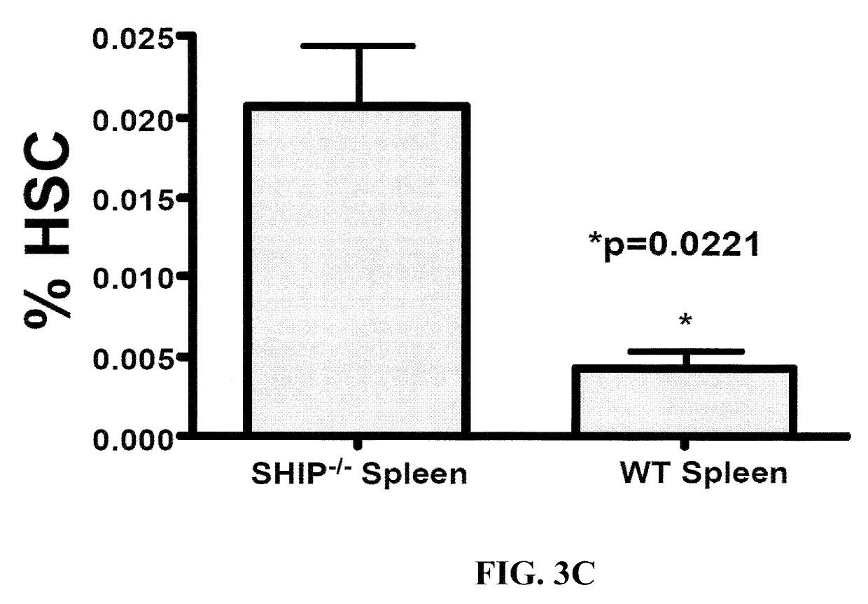

SEQ ID NO:2 is the mouse SHIP cDNA sequence (GenBank accession no. NM_{—}010566) (FIG. 3).

SEQ ID NOs:3-15 are examples of target regions within the human SHIP open reading frame.

SEQ ID NOs:16-17 are siRNA sequences (designated H1 and H2, respectively) that have been identified as effective at SHIP knockdown in human NK cell lines.

SEQ ID NOs:18-19 are shRNA sequences (designated 63332 and 63331, respectively) targeting human SHIP, including the loop and termini portions of the shRNA.

SEQ ID NOs:20-21 are the target sequences within human SHIP for shRNA 6332 (SEQ ID NO:18) and shRNA 63331 (SEQ ID NO:19), respectively.

### DETAILED DESCRIPTION OF THE INVENTION

The present invention concerns inhibition of SHIP expression and/or function in mammalian subjects to direct hematopoietic stem cells (HSC) from the bone marrow to other sites in the body (such as the spleen and/or liver), where they then re-establish blood cell production. Preferably, inhibition of SHIP expression and/or function is achieved by administering an effective amount of one or more SHIP inhibitors to the subject. Preferably, the SHIP inhibitor(s) is administered to the subject intravenously.

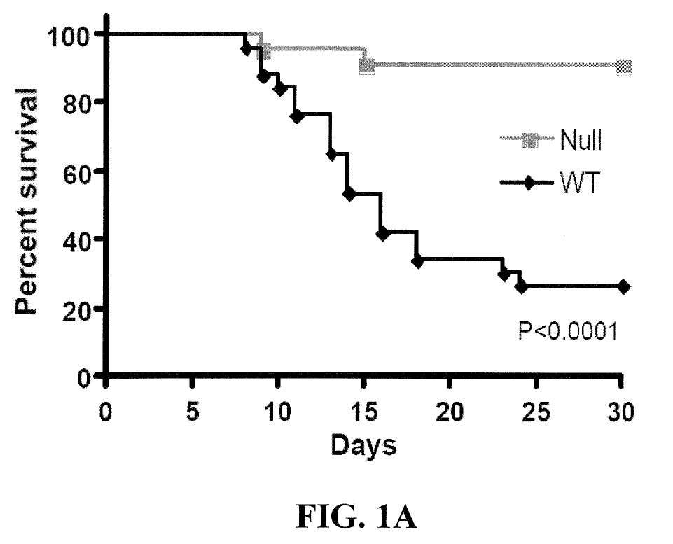

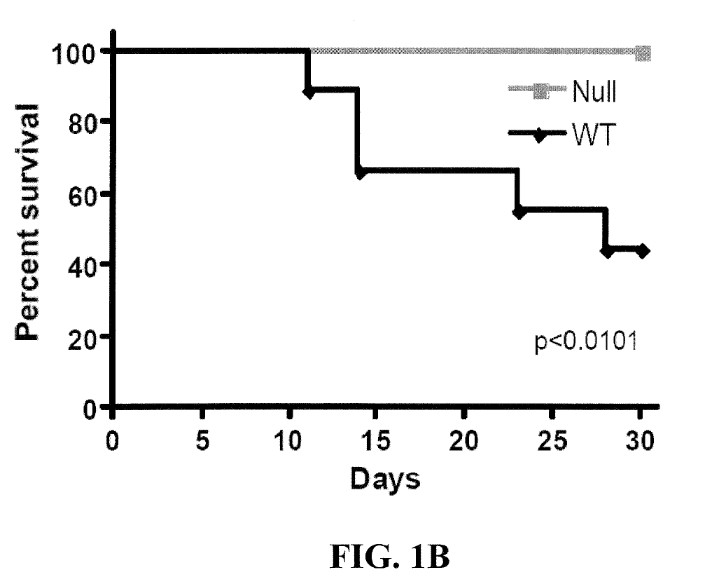

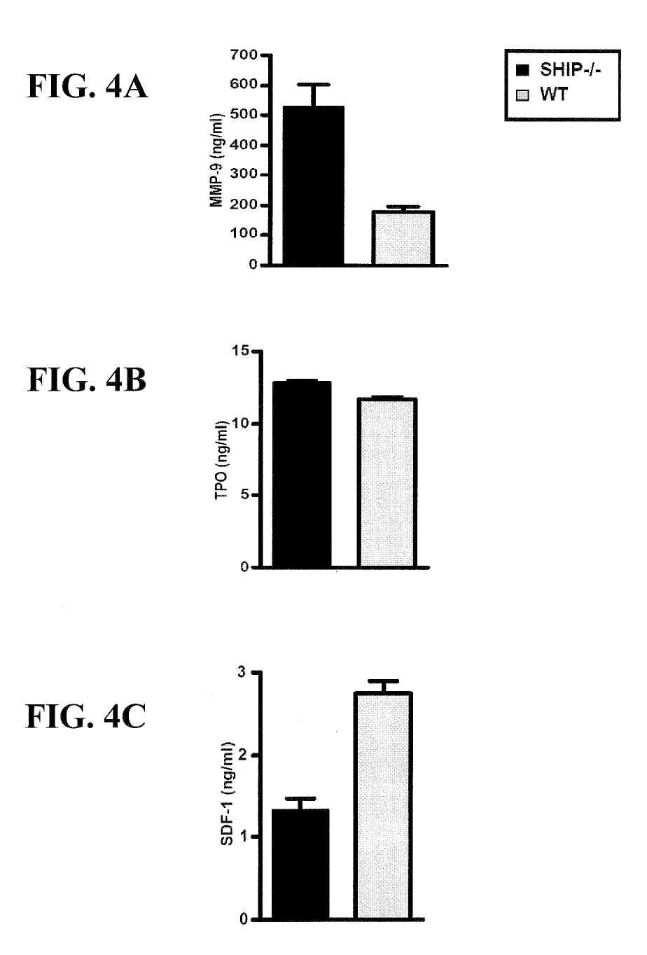

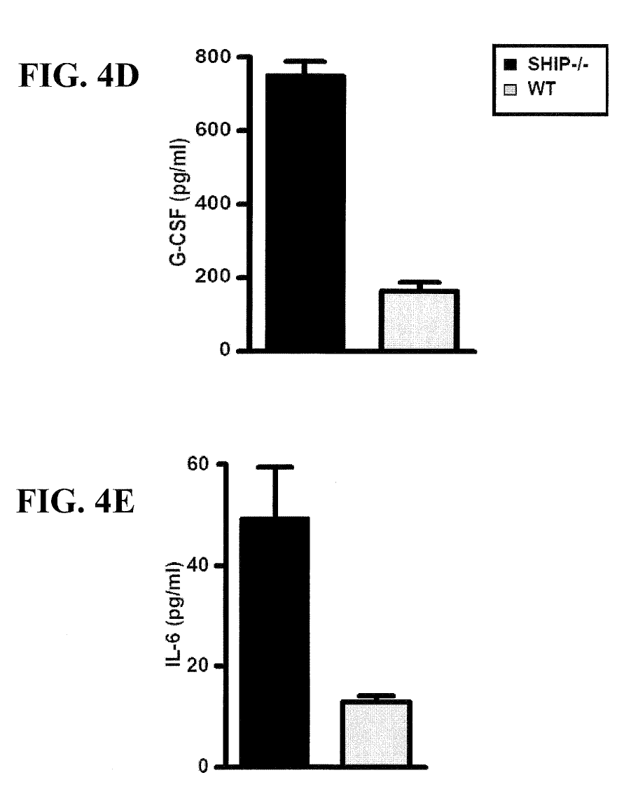

FIGS. 1A and 1B show that cells from the spleen of SHIP-deficient mice are enriched for cells with radioprotective capacity. FIGS. 2A-2D show that HSC SHIP-deficient spleens are capable of long-term blood compartment reconstitution. FIGS. 3A-3C show that spleens of SHIP-deficient mice contain cells capable of renewing the splenic HSC compartment. Thus, SHIP−/− spleens contain significant radioprotective and long-term blood cell repopulating activity—two properties of the HSC. That these activities are transplantable demonstrates that they are present in the spleen of SHIP-deficient mice and, therefore, this can allow individuals rendered SHIP-deficient to support HSC function in their spleen. FIGS. 4A-4E show that cytokine serum levels are increased in SHIP-deficient mice, indicating a disrupted marrow microenvironment. It is this disrupted environment that could be triggering the mobilization of stem cells to the periphery.

The methods of the invention can be employed, for example, to rescue or enhance blood cell recovery during bone marrow impairment (e.g., complete or partial bone marrow failure), such as due to bone-seeking radioisotope exposure, chemotherapy, radiotherapy, infection, or genetic or congenital defects. Bone marrow failure syndromes that may be treated by the methods of the invention include a group of disorders that can be either inherited or acquired. These disorders can involve one or more blood cell lines (erythroid for red cells, myeloid for white blood cells, and/or megakaryocytic for platelets). The pathophysiology of these hematopoietic defects can include a decrease in or damage to the hematopoietic stem cells and their microenvironment, resulting in hypoplastic or aplastic bone marrow; maturation defects (e.g., vitamin B-12 or folate deficiency); and/or differentiation defects (e.g., myelodysplasia). Generally, the HSC are damaged by a congenital defect or exposure to a noxious substance or factor (e.g., acquired stem cell injury from viruses, toxins, or chemicals that lead to a quantitative or qualitative abnormality; abnormal humoral or cellular control of hematopoiesis; abnormal or hostile marrow microenvironment; immunologic suppression of hematopoiesis (e.g., mediated by antibodies, T cells (or cellularly), or lymphokines); and mutations in genes, causing inherited bone marrow failure syndromes); see, for example, Bone Marrow Failure Syndromes, edited by Neal S. Young, Philadelphia, W.B. Saunders, 2000; Chen, 2005; Freedman, 1996; Livingston et al., 2003; and Dritschilo and Sherman, 1981).

Examples of inherited bone marrow failure syndromes that may be treated by the methods of the invention include, for example, Fanconi anemia, dyskeratosis congenital, Diamond-Blackfan anemia, Schwachman-Diamond syndrome, and other genetic disorders (Alter B. P., Inherited bone marrow failure syndromes, In: Nathan D. B. et al., Nathan and Oski's Hematology of Infancy and Childhood, 6^{th} edition, Philadelphia, Pa., WB Saunders, 2003:280-365; and Alter B. P., Inherited forms of aplastic anemia: the inherited bone marrow failure syndromes (IBMFS), In: Burg F. D. et al., Gellis and Kagan's Current Pediatric Therapy, Philadelphia, Elsevier, 2005). Hypoproliferative anemias associated with marrow damage include aplastic anemia, myelodysplasia (MDS), pure red cell aplasia (PRCA), and myelophthisis. In bone marrow failure, pancytopenia-anemia, leucopenia, and/or thrombocytopenia results from deficient hematopoiesis, as distinguished from blood count depression due to peripheral destruction of red cells (hemolytic anemias), platelets, and granulocytes.

In one aspect, the present invention concerns a method for treating or delaying the onset of impaired bone marrow function (including partial or complete bone marrow failure, acute or chronic impairment of bone marrow function, and congenital or acquired impairment of bone marrow function) in a subject, comprising administering an effective amount of a SHIP inhibitor to the subject. In one embodiment, the impaired bone marrow function is an impaired bone marrow function syndrome such an aplastic bone marrow failure syndrome. Optionally, prior to the administration of the SHIP inhibitor, the method can further comprise the step of identifying the subject as one suffering from impaired bone marrow function, or at risk of developing impaired bone marrow function.

In another aspect, the present invention concerns a method for directing HSC from the bone marrow in a subject exposed to, or at risk of exposure to, a bone-seeking radioisotope (such as Strontium 90, the radioisotope of choice for “dirty bombs”). Thus, the method of the invention can be used to treat or delay the onset of radiation injury manifesting as impaired bone marrow function. Optionally, prior to administration of the SHIP inhibitor, the method can further comprise the step of identifying the subject as one suffering from radiation injury, exposure to a bone-seeking radioisotope, and/or suffering from impaired bone marrow function caused by exposure to a bone-seeking radioisotope; or identifying the subject as one at risk of radiation exposure, at risk of exposure to a bone-seeking radioisotope, or at risk of developing impaired bone marrow function caused by exposure to a bone-seeking radioisotope. The exposure to the radioactive material may be accidental (e.g., industrial), or intentional (e.g., terrorism). In addition to Strontium 90, other examples of bone seeking radioisotopes can be found, for example, in Colella et al., 2005; CDC Fact Sheet (2003), Acute Radiation Syndrome, Center for Disease Control and Prevention, U.S. Department of Health and Human Services; Edwards, R. Risk of radioactive “dirty bomb” growing, New Scientist online, June 2004; and Chapter XII-Biological Effects, In: The Effects of Nuclear Weapons, Third Edition, compiled and edited by Glasstone S, and P. J. Dolan, United States Department of Defense and the Energy Research and Development Administration, 1977.

In another aspect, the present invention concerns a method for treating or delaying the onset of fibrotic and/or damaged bone marrow due to chemotherapy and/or radiation therapy for cancer. Optionally, the method of the invention can be utilized with one or more other treatments, such as treatments intended to increase hematopoietic function (e.g., erythropoietin (EPO), filgrastim (Neupogen), lenograstim (Granocyte), and pegylated filgrastim (Neulasta)). Optionally, prior to administration of the SHIP inhibitor, the method of the invention can further comprise identifying the subject as one suffering from fibrotic and/or damaged bone marrow due to chemotherapy and/or radiation therapy, or identifying the subject as one at risk of suffering from fibrotic and/or damaged bone marrow due to chemotherapy and/or radiation therapy.

Desponts C. et al. (2006a) and U.S. Patent Application Publication No. 2006/0223749 (Desponts, et al., “Inhibition of SHIP to enhance stem cell harvest and transplantation”) are each incorporated herein by reference in their entirety.

As used herein, the term “SHIP” refers to hematopoietic-specific SH2-containing inositol-5-phosphatase-1, which catalyzes the removal of the 5′ phosphate group from PI_{(3,4,5)}P3 (PIPS) and inositol 1,3,4,5-tetrakisphosphate (IP4). SHIP, which is also known in the scientific literature as SHIP-1, SHIP1, SHIPI, and SHIP-I was also the subject of Helgason et al., 1998; Huber et al., 1998a; Liu et al., 1999; Liu et al., 1998; Rohrschneider et al., 2000, U.S. Pat. No. 6,090,621 (Kavanaugh et al.), PCT publication WO 9710252A1 (Rohrschneider, L. R.), and PCT publication WO 9712039A2 (Krystal, G.). The nucleotide sequences of mouse SHIP and human SHIP, for example, have been publicly available for several years (GenBank Accession Numbers NM_{—}10566 and NM_{—}005541, respectively, on the National Center for Biotechnology Information (NCBI) database).

The terms “inhibitor of SHIP”, “SHIP inhibitor”, and “SHIP inhibiting substance” are used herein interchangeably to refer to any molecule that decreases the activity of SHIP (inositol phosphatase activity) or decreases the protein level of SHIP. Thus, a SHIP inhibitor can be a small molecule (e.g., having low molecular weight) that decreases activity of SHIP, e.g., by interfering with interaction of the inositol phosphatase with another molecule, e.g., its substrate. The SHIP inhibitor can also be a small molecule that decreases expression of the gene encoding the inositol phosphatase. An inhibitor can also be an interfering RNA molecule, antisense oligonucleotide, a ribozyme, an antibody, or a dominant negative mutant of SHIP. Dominant negative mutants of SHIP have been developed (Gupta et al., 1997; and Tridandapani et al., 2002, which are each incorporated herein by reference in their entirety). A “direct inhibitor” of SHIP is an inhibitor that interacts with the SHIP enzyme, or substrate thereof, or with a nucleic acid encoding SHIP (e.g., the SHIP gene or its mRNA) or its regulatory sequences. An “indirect inhibitor” of SHIP is an inhibitor that interacts upstream or downstream of the SHIP enzyme in the regulatory pathway, and which does not interact with the enzyme or substrate thereof or with a nucleic acid encoding SHIP or its regulatory sequences.

As used herein, the term “polypeptide” refers to any polymer comprising any number of amino acids, and is interchangeable with “protein”, “gene product”, and “peptide”.

As used herein, the term “nucleoside” refers to a molecule having a purine or pyrimidine base covalently linked to a ribose or deoxyribose sugar. Exemplary nucleosides include adenosine, guanosine, cytidine, uridine and thymidine.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms. The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

As used herein, the term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference or otherwise reduce target gene expression.

The terms “operably-linked” or “operatively-linked” are used herein interchangeably to refer to an arrangement of flanking sequences wherein the flanking sequences so described are configured or assembled so as to perform their usual function. Thus, a flanking sequence operably-linked to a coding sequence may be capable of effecting the replication, transcription and/or translation of the coding sequence. For example, a coding sequence is operably-linked to a promoter when the promoter is capable of directing transcription of that coding sequence. A flanking sequence need not be contiguous with the coding sequence, so long as it functions correctly. Thus, for example, intervening untranslated yet transcribed sequences can be present between a promoter sequence and the coding sequence, and the promoter sequence can still be considered “operably-linked” to the coding sequence. Each nucleotide sequence coding for a siRNA will typically have its own operably-linked promoter sequence.

The term “vector” is used to refer to any molecule (e.g., nucleic acid, plasmid, or virus) used to transfer coding information (e.g., a polynucleotide of the invention) to a host cell. The terms “expression vector” and “transcription vector” are used interchangeably to refer to a vector that is suitable for use in a host cell (e.g., a subject's cell) and contains nucleic acid sequences that direct and/or control the expression of exogenous nucleic acid sequences. Expression includes, but is not limited to, processes such as transcription, translation, and RNA splicing, if introns are present.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of endogenous target genes, such as SHIP.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a siRNA having a “sequence sufficiently complementary to a target mRNA sequence to direct target-specific RNA interference (RNAi)” means that the siRNA has a sequence sufficient to trigger the destruction of the target mRNA (e.g., SHIP mRNA) by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

As used herein, the term “cleavage site” refers to the residues, e.g., nucleotides, at which RISC* cleaves the target RNA, e.g., near the center of the complementary portion of the target RNA, e.g., about 8-12 nucleotides from the 5′ end of the complementary portion of the target RNA.

The term “dominant negative mutant” is art-recognized and refers to the mutant form of a wild-type protein that interferes with the function of the wild-type protein (e.g., by interacting with the wild-type protein). Thus, overexpression of the dominant negative mutant can be expected to interfere with the function of the wild-type version of the protein (in this case, SHIP).

As used herein, the term “mismatch” refers to a basepair consisting of noncomplementary bases, e.g., not normal complementary G:C, A:T or A:U base pairs.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

As used herein, the term “in vitro” has its art recognized meaning, e.g., involving purified reagents or extracts, e.g., cell extracts. The term “in vivo” also has its art recognized meaning, e.g., involving living cells in an organism, e.g., immortalized cells, primary cells, and/or cell lines, in an organism.

A gene “involved in” or “associated with” a disorder includes a gene, the normal or aberrant expression or function of which affects or causes a disease or disorder or at least one symptom of the disease or disorder. The methods of the invention are useful in inducing mobilization of hematopoietic stem cells and hematopoietic progenitor cells and extramedullary hematopoiesis. Thus, the methods of the invention are useful in the treatment of human or non-human animal subjects suffering from, or at risk of developing, disorders associated with impaired bone marrow function.

The methods of the invention may include further steps. In some embodiments, a subject with the relevant condition or disease (e.g., disorders associated with impaired bone marrow function (e.g., hematopoiesis)) is identified or a patient at risk for the condition or disease is identified prior to administration of the SHIP inhibitor. A patient may be someone who has not been diagnosed with the disease or condition (diagnosis, prognosis, and/or staging) or someone diagnosed with the disease or condition (diagnosis, prognosis, monitoring, and/or staging), including someone treated for the disease or condition (prognosis, staging, and/or monitoring). Alternatively, the person may not have been diagnosed with the disease or condition but suspected of having the disease or condition based either on patient history or family history, or the exhibition or observation of characteristic symptoms.

As used herein, an “effective amount” of a SHIP inhibitor (such as an interfering RNA, an antisense oligonucleotide, or a ribozyme, which selectively interferes with expression of SHIP) is that amount effective to bring about the physiological changes desired in the cells to which the SHIP inhibitor is administered in vitro (e.g., ex vivo) or in vivo. The term “therapeutically effective amount” as used herein, means that amount of SHIP inhibitor alone or in combination with another agent according to the particular aspect of the invention, that elicits the biological or medicinal response in cells (e.g., tissue(s)) that is being sought by a researcher, veterinarian, medical doctor or other clinician, which includes alleviation and/or prevention of the symptoms of the disease or disorder being treated. Preferably, suppression of SHIP function (e.g., by reduction of SHIP expression) results in mobilization of hematopoietic stem cells and/or hematopoietic progenitor cells from the bone marrow to another site(s) in the body (e.g., spleen and/or liver) and re-establishment of blood cell production at that site or sites.

Various methods of the present invention can include a step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Reduction (suppression) of expression results in a decrease of SHIP mRNA and/or protein. For example, in a given cell, the suppression of SHIP mRNA by administration of a SHIP inhibitor that reduces SHIP function by reducing SHIP expression (such as interfering RNA, antisense oligonucleotide, or ribozyme) results in a decrease in the quantity of SHIP mRNA relative to an untreated cell. Suppression may be partial. Preferred degrees of suppression are at least 50%, more preferably one of at least 60%, 70%, 80%, 85%, or 90%. A level of suppression between 90% and 100% is generally considered a “silencing” of expression. Where shunting of HSC from the bone marrow is desired, the level of suppression is sufficient to cause mobilization of HSC from the bone marrow to another site of the body (e.g., spleen and/or liver) and, preferably, extramedullary hematopoiesis.

SHIP gene expression can be determined before and/or after introduction of the SHIP inhibitor in vitro or in vivo. Reduction in SHIP gene expression can be detected at either the protein or mRNA level. Protein expression analysis can be performed by Western blotting, immunofluorescence, or flow cytometry and cell sorting (FACS). Reduction in SHIP gene expression can be detected at the mRNA level by real-time RT-PCR, microarray analysis, or Northern blotting, for example. Preferably, all expression data is compared with levels of a “house keeping” gene to normalize for variable amounts of RNA in different samples.

### RNA Interference

A naturally occurring gene-silencing mechanism triggered by double-stranded RNA (dsRNA), designated as small interfering RNA (siRNA), has emerged as a very important tool to suppress or knock down gene expression in many systems. RNA interference is triggered by dsRNA that is cleaved by an RNAse-III-like enzyme, Dicer, into 21-25 nucleotide fragments with characteristic 5′ and 3′ termini (Provost et al., 2002). These siRNAs act as guides for a multi-protein complex, including a PAZ/PIWI domain containing the protein Argonaute2, that cleaves the target mRNA (Hammond et al., 2001). These gene-silencing mechanisms are highly specific and potent and can potentially induce inhibition of gene expression throughout an organism. The short interference RNA (siRNA) approach has proven effective in silencing a number of genes of different viruses (Fire, 1999).

RNA interference (RNAi) is a polynucleotide sequence-specific, post-transcriptional gene silencing mechanism effected by double-stranded RNA that results in degradation of a specific messenger RNA (mRNA), thereby reducing the expression of a desired target polypeptide encoded by the mRNA (see, e.g., WO 99/32619; WO 01/75164; U.S. Pat. No. 6,506,559; Fire et al., 1998; Sharp, 1999; Elbashir et al., 2001c; Harborth et al., 2001). RNAi is mediated by double-stranded polynucleotides, such as double-stranded RNA (dsRNA), having sequences that correspond to exonic sequences encoding portions of the polypeptides for which expression is compromised. RNAi reportedly is not effected by double-stranded RNA polynucleotides that share sequence identity with intronic or promoter sequences (Elbashir et al., 2001c). RNAi pathways have been best characterized in Drosophila and Caenorhabditis elegans, but “small interfering RNA” (siRNA) polynucleotides that interfere with expression of specific polynucleotides in higher eukaryotes such as mammals (including humans) have also been considered (e.g., Tuschl, 2001; Sharp, 2001; Bernstein et al., 2001a; Zamore, 2002; Plasterk, 2002; Zamore, 2001; Matzke et al., 2001; Scadden et al., 2001).

According to a current non-limiting model, the RNAi pathway is initiated by ATP-dependent cleavage of long dsRNA into double-stranded fragments of about 18-27 (e.g., 19, 20, 21, 22, 23, 24, 25, 26, etc.) nucleotide base pairs in length, called small interfering RNAs (siRNAs) (see review by Hutvagner et al., 2002; Elbashir et al., 2001c; Nyknen et al., 2001; Zamore et al., 2000). In Drosophila, an enzyme known as “Dicer” cleaves the longer double-stranded RNA into siRNAs; Dicer belongs to the RNase III family of dsRNA-specific endonucleases (WO 01/68836; Bernstein et al., 2001b). Further, according to this non-limiting model, the siRNA duplexes are incorporated into a protein complex, followed by ATP-dependent unwinding of the siRNA, which then generates an active RNA-induced silencing complex (RISC) (WO 01/68836). The complex recognizes and cleaves a target RNA that is complementary to the guide strand of the siRNA, thus interfering with expression of a specific protein (Hutvagner et al., 2002).

In C. elegans and Drosophila, RNAi may be mediated by long double-stranded RNA polynucleotides (WO 99/32619; WO 01/75164; Fire et al., 1998; Clemens et al., 2000; Kisielow et al., 2002; see also WO 01/92513 (RNAi-mediated silencing in yeast)). In mammalian cells, however, transfection with long dsRNA polynucleotides (i.e., greater than 30 base pairs) leads to activation of a non-specific sequence response that globally blocks the initiation of protein synthesis and causes mRNA degradation (Bass, 2001). Transfection of human and other mammalian cells with double-stranded. RNAs of about 18-27 nucleotide base pairs in length interferes in a sequence-specific manner with expression of particular polypeptides encoded by messenger RNAs (mRNA) containing corresponding nucleotide sequences (WO 01/75164; Elbashir et al., 2001c; Elbashir et 2001b; Harborth et al., 2001; Carthew et al., 2001; Mailand et al., 2002).

siRNA may offer certain advantages over other polynucleotides known to the art for use in sequence-specific alteration or modulation of gene expression to yield altered levels of an encoded polypeptide product. These advantages include lower effective siRNA polynucleotide concentrations, enhanced siRNA stability, and shorter siRNA oligonucleotide lengths relative to such other polynucleotides (e.g., antisense, ribozyme or triplex polynucleotides). By way of a brief background, “antisense” polynucleotides bind in a sequence-specific manner to target nucleic acids, such as mRNA or DNA, to prevent transcription of DNA or translation of the mRNA (see, e.g., U.S. Pat. No. 5,168,053; U.S. Pat. No. 5,190,931; U.S. Pat. No. 5,135,917; U.S. Pat. No. 5,087,617; see also, e.g., Clusel et al., 1993, describing “dumbbell” antisense oligonucleotides). “Ribozyme” polynucleotides can be targeted to an RNA transcript and are capable of catalytically cleaving such transcripts, thus impairing translation of mRNA (see, e.g., U.S. Pat. No. 5,272,262; U.S. Pat. No. 5,144,019; and U.S. Pat. Nos. 5,168,053, 5,180,818, 5,116,742 and 5,093,246; U.S. Ser. No. 2002/193579). “Triplex” DNA molecules refers to single DNA strands that bind duplex DNA to form a colinear triplex molecule, thereby preventing transcription (see, e.g., U.S. Pat. No. 5,176,996, describing methods for making synthetic oligonucleotides that bind to target sites on duplex DNA). Such triple-stranded structures are unstable and form only transiently under physiological conditions. Because single-stranded polynucleotides do not readily diffuse into cells and are therefore susceptible to nuclease digestion, development of single-stranded DNA for antisense or triplex technologies often requires chemically modified nucleotides to improve stability and absorption by cells. siRNAs, by contrast, are readily taken up by intact cells, are effective at interfering with the expression of specific polynucleotides at concentrations that are several orders of magnitude lower than those required for either antisense or ribozyme polynucleotides, and do not require the use of chemically modified nucleotides.

Due to its advantages, RNAi has been applied as a target validation tool in research and as a potential strategy for in vivo target validation and therapeutic product development (Novina and Sharp, 2004). In vivo gene silencing with RNAi has been reported using viral vector delivery and high-pressure, high-volume intravenous (i.v.) injection of synthetic iRNAs (Scherr et al., 2003c; Song et al., 2003). In vivo gene silencing has been reported after local direct administration (intravitreal, intranasal, and intrathecal) of siRNAs to sequestered anatomical sites in various models of disease or injury, demonstrating the potential for delivery to organs such as the eye, lungs, and central nervous system (Reich et al., 2003; Zhang et al., 2004; Dorn et al., 2004). Silencing of endogenous genes by systemic administration of siRNAs has also been demonstrated (Soutschek et al., 2004). It has been shown that siRNAs delivered systemically in a liposomal formulation can silence the disease target apolipoprotein B (ApoB) in non-human primates (Zimmermann et al., 2006).

As indicated above, RNAi is an efficient process whereby double-stranded RNA (dsRNA, also referred to herein as siRNAs or ds siRNAs, for double-stranded small interfering RNAs) induces the sequence-specific degradation of targeted mRNA in animal and plant cells (Hutvagner and Zamore, 2002; Sharp, 2001). In mammalian cells, RNAi can be triggered by 21-nucleotide (nt) duplexes of small interfering RNA (siRNA) (Chiu et al., 2002; Elbashir et al., 2001c), or by micro-RNAs (miRNA), functional small-hairpin RNA (shRNA), or other dsRNAs which can be expressed in vivo using DNA templates with RNA polymerase III promoters (Zeng et al., 2002; Paddison et al., 2002; Lee et al., 2002; Paul et al., 2002; Tuschl, 2002; Yu et al., 2002; McManus et al., 2002; Sui et al., 2002), each of which are incorporated herein by reference in their entirety.

The scientific literature contains many reports of endogenous and exogenous gene expression silencing using siRNA, highlighting their therapeutic potential (Gupta et al., 2004; Takaku, 2004; Pardridge, 2004; Zheng, 2004; Shen, 2004; Fuchs et al., 2004; Wadhwa et al., 2004; Ichim et al., 2004; Jana et al., 2004; Ryther et al., 2005; Chae et al., 2004; Fougerolles et al., 2005), each of which is incorporated herein by reference in its entirety. Therapeutic silencing of endogenous genes by systemic administration of siRNAs has been described in the literature (Kim B. et al., 2004; Soutschek et al., 2004; Pardridge, 2004), each of which is incorporated herein by reference in its entirety.

Accordingly, the invention includes such interfering RNA molecules that are targeted to SHIP mRNA. The interfering RNA molecules are capable, when suitably introduced into or expressed within a cell that otherwise expresses SHIP mRNA, of suppressing expression of the SHIP gene by RNAi. The interfering RNA may be a double-stranded siRNA. As the skilled person will appreciate, and as explained further herein, an siRNA molecule may include a short 3′ DNA sequence also. Alternatively, the nucleic acid may be a DNA (usually double-stranded DNA) which, when transcribed in a cell, yields an RNA having two complementary portions joined via a spacer, such that the RNA takes the form of a hairpin when the complementary portions hybridize with each other. In a mammalian cell, the hairpin structure may be cleaved from the molecule by the enzyme Dicer, to yield two distinct, but hybridized, RNA molecules.

In one embodiment, the invention provides an interfering RNA that is capable, when suitably introduced or expressed within a cell that normally expresses SHIP mRNA, suppresses its expression by RNAi, wherein the interfering RNA is generally targeted to the SHIP enzymatic domain (inositol 5′-phosphatase domain), within the human SHIP cDNA (SEQ ID NO:1). Examples of SHIP target sequences include GCCTGTTGTCATCCATTGA (SEQ ID NO:3), ATAAGTTGGTGATCTTGGT (SEQ ID NO:4), GCCACATCTGTACTGACAA (SEQ ID NO:5), AGACAGGCATTGCAAACAC (SEQ ID NO:6), ACATCACTCACCGCTTCAC (SEQ ID NO:7), TCTTAACTACCGTGTGGAT (SEQ ID NO:8), AATACGCCTACACCAAGCA (SEQ ID NO:9), GTACCAGCGACATCATGAC (SEQ ID NO:10), GCGACATCATGACGAGTGA (SEQ ID NO:11), AGGACAGATTGAGTTTCTC (SEQ ID NO:12), GGTGCTATGCCACATTGAA (SEQ ID NO:13), GTTTGGTGAGACTCTTCCA (SEQ ID NO:14), AGACGGAGCGTGATGAATC (SEQ ID NO:15), GCTTCCAGAAGAGCATCTTAT (SEQ ID NO:20), and GCCCATATCACCCAAGAAGTTT (SEQ ID NO:21). In a specific embodiment, the interfering RNA comprises a sequence selected from the group consisting of SEQ ID NO:16, SEQ ID NO:17, SEQ ID NO:18, and SEQ ID NO:19. Preferably, the interfering RNA sequence is within the range of about 19 to 23 nucleotides. For example, in those embodiments in which an shRNA is utilized, that portion of the shRNA targeting SHIP is preferably within the range of about 19 to 23 nucleotides.

It is expected that perfect identity/complementarity between the interfering RNA used in the method of the invention and the target sequence, although preferred, is not essential. Accordingly, the interfering RNA may include a single mismatch compared to the target sequence within the SHIP mRNA. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

siRNA Molecules

Short interfering RNAs (siRNAs) induce the sequence-specific suppression or silencing (i.e., reducing expression which may be to the extent of partial or complete inhibition) genes by the process of RNAi. Thus, siRNA is the intermediate effector molecule of the RNAi process. The interfering RNA that function as SHIP inhibitors include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the SHIP mRNA, and the other strand is identical or substantially identical to the first strand. The dsRNA molecules that function as SHIP inhibitors can be chemically synthesized, or can be transcribed in vitro from a DNA template, or in vivo from, e.g., shRNA. The dsRNA molecules can be designed using any method known in the art, for instance, by using the following protocol:

1. Using any method known in the art, compare the potential targets to the appropriate genome database (human, mouse, rat, etc.) and eliminate from consideration any target sequences with significant homology to other coding sequences. One such method for sequence homology searches is known as BLAST, which is available at the National Center for Biotechnology Information (NCBI) web site of the National Institutes of Health. Also available on the NCBI webs site is the HomoloGene database, which is a publicly available system for automated detection of homologs among the annotated genes of several completely sequenced eukaryotic genomes and is readily utilized by those of ordinary skill in the art.

2. Select one or more sequences that meet your criteria for evaluation. Further general information regarding the design and use of siRNA can be found in “The siRNA User Guide,” available at the web site of the laboratory of Dr. Thomas Tuschl at Rockefeller University (Elbashir et al., 2001a).

3. Negative control siRNAs preferably have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

Initially, basic criteria were defined for identification of efficient siRNA, such as GC content and position of the targeted sequence in the context of the mRNA (Elbashir S M. et al., 2002). Further progress was achieved more recently, when the assembly of the RNAi enzyme complex was described as being dependent on thermodynamic characteristics of the siRNA (Khrvorova et al., 2003; Schwarz et al., 2003). The relative stability of both ends of the duplex was determined to have effects on the extent to which the individual strands enter the RNAi pathway. In addition, certain sequence motifs at defined positions of the siRNA were reported to influence its potency (Amarzguioui and Prydz, 2004; Reynolds et al., 2004). On this basis, sophisticated algorithms have been developed to increase the success rate of siRNA design and are available to those skilled in the art (Amarzguioui and Prydz, 2004; Reynolds et al., 2004; and Ui-Tei et al., 2004, each of which is incorporated herein in its entirety).

Other computational tools that may be used to select siRNAs of the present invention include the Whitehead siRNA selection Web Server from the bioinformatics group at the Whitehead Institute for Biomedical Research in Cambridge, Mass., and other disclosed in Yuan, B. et al. (2004) and Bonetta L. (2004), each of which are incorporated by reference herein in their entirety.

The efficiencies of different siRNAs may differ significantly. However, strategies for rational design of effective interfering RNA exist (Gong and Ferrell Jr., 2004; Schubert et al., 2005; Pancoska et al., 2004; Mittal, 2004, each of which is incorporated herein by reference in its entirety).

Screening for the most efficient siRNAs using cell cultures may be carried out. Several in vitro screening methods based on the use of siRNA mixtures, which may contain a particular efficient siRNA (or several), have been developed. These include the preparation of siRNA mixtures using RNase III or Dicer enzymes to digest longer double-stranded RNAs, such as BLOCK-IT products (INVITROGEN, Carlsbad Calif.) (Yang et al., 2002; Myers et al., 2003). The short RNAs produced as a result of these digestions have been found to be efficient in RNAi. Oligonucleotide arrays can also be used for the efficient preparation of defined mixtures of siRNAs for reducing the expression of exogenous and endogenous genes such as SHIP (Oleinikov et al., 2005).

The SHIP inhibitors of the invention can include both unmodified siRNAs and modified siRNAs as known in the art. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′ OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The SHIP inhibitors of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al. (2001) (describes nucleic acids loaded to polyalkylcyanoacrylate (PACA) nanoparticles); Fattal et al. (1998) (describes nucleic acids bound to nanoparticles); Schwab et al. (1994) (describes nucleic acids linked to intercalating agents, hydrophobic groups, polycations or PACA nanoparticles); and Godard et al. (1995) (describes nucleic acids linked to nanoparticles).

The SHIP inhibitors of the present invention can also be labeled using any method known in the art; for instance, nucleic acids can be labeled with a fluorophore, e.g., Cy3, fluorescein, or rhodamine. The labeling can be carried out using a kit, e.g., the SILENCER siRNA labeling kit (AMBION). Additionally, the siRNA can be radiolabeled, e.g., using ³H, ³²P, or other appropriate isotope.

Because RNAi is believed to progress via at least one single stranded RNA intermediate, the skilled artisan will appreciate that ss-siRNAs (e.g., the antisense strand of a ds-siRNA) can also be designed as described herein and utilized according to the claimed methodologies.

There are a number of companies that will generate interfering RNAs for a specific gene. Thermo Electron Corporation (Waltham, Mass.) has launched a custom synthesis service for synthetic short interfering RNA (siRNA). Each strand is composed of 18-20 RNA bases and two DNA bases overhang on the 3′ terminus. Dharmacon, Inc. (Lafayette, Colo.) provides siRNA duplexes using the 2′-ACE RNA synthesis technology. Qiagen (Valencia, Calif.) uses TOM-chemistry to offer siRNA with high individual coupling yields (Li et al., 2005).

siRNA Delivery for Longer-Term Expression

Synthetic siRNAs can be delivered into cells by methods known in the art, including cationic liposome transfection (LIPOFECTAMINE 2000 reagent, for example) and electroporation, for example. However, these exogenous siRNA generally show short term persistence of the silencing effect (4 to 5 days in cultured cells), which may be beneficial in certain embodiments. To obtain longer suppression of SHIP expression and to facilitate delivery under certain circumstances, one or more siRNA duplexes, e.g., SHIP ds siRNA, can be expressed within cells from recombinant DNA constructs (McIntyre and Fanning, 2006). Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl, 2002) capable of expressing functional double-stranded siRNAs; (Bagella et al., 1998; Lee et al., 2002; Miyagishi et al. 2002; Paul et al., 2002; Yu et al., 2002; Sui et al., 2002). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression (Bagella et al., 1998; Lee et al., 2002; Miyagishi et al., 2002; Paul et al., 2002; Yu et al., 2002; Sui et al., 2002). Constructs containing siRNA sequence(s) under the control of a T7 promoter also make functional siRNAs when co-transfected into the cells with a vector expressing T7 RNA polymerase (Jacque, 2002). A single construct may contain multiple sequences coding for siRNAs, such as multiple regions of SHIP mRNA, and can be driven, for example, by separate PolIII promoter sites.

Animal cells express a range of noncoding RNAs of approximately 22 nucleotides termed micro RNA (miRNAs) which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng, 2002). When expressed by DNA vectors containing polymerase III promoters, micro-RNA designed hairpins can silence gene expression (McManus, 2002). Viral-mediated delivery mechanisms can also be used to induce specific silencing of targeted genes through expression of siRNA, for example, by generating recombinant adenoviruses harboring siRNA under RNA Pol II promoter transcription control (Xia et al., 2002). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. In an animal model, whole-embryo electroporation can efficiently deliver synthetic siRNA into post-implantation mouse embryos (Calegari et al., 2002). In adult mice, efficient delivery of siRNA can be accomplished by the “high-pressure” delivery technique, a rapid injection (within 5 seconds) of a large volume of siRNA containing solution into animal via the tail vein (Liu, 1999; McCaffrey, 2002; Lewis, 2002). Nanoparticles, liposomes and other cationic lipid molecules can also be used to deliver siRNA into animals. It has been shown that siRNAs delivered systemically in a liposomal formulation can silence the disease target apolipoprotein B (ApoB) in non-human primates (Zimmermann et al., 2006). A gel-based agarose/liposome/siRNA formulation is also available (Jiamg et al., 2004).

### Uses of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the SHIP mRNA to be targeted by the siRNA generated from the engineered. RNA precursor will be depleted from the cell or organism, leading to a decrease in the concentration of any translational product encoded by that mRNA in the cell or organism. The RNA precursors are typically nucleic acid molecules that individually encode either one strand of a dsRNA or encode the entire nucleotide sequence of an RNA hairpin loop structure.

### Antisense

An “antisense” nucleic acid sequence (antisense oligonucleotide) can include a nucleotide sequence that is complementary to a “sense” nucleic acid encoding a protein, e.g., complementary to the coding strand of a double-stranded cDNA molecule or complementary to the SHIP mRNA. Antisense nucleic acid sequences and delivery methods are well known in the art (Goodchild, 2004; Clawson et al., 2004), which are incorporated herein by reference in their entirety. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence within the SHIP mRNA. An antisense oligonucleotide can be, for example, about 7, 10, 15, 20, 25, 30, 35, 40, 45, 50, 55, 60, 65, 70, 75, 80, or more nucleotides in length.

An antisense nucleic acid sequence can be designed such that it is complementary to the entire SHIP mRNA sequence, but can also be an oligonucleotide that is antisense to only a portion of the SHIP mRNA. For example, the antisense oligonucleotide can be complementary to a portion of the SHIP enzymatic domain (inositol 5′-phosphatase domain) or a portion of the amino-terminal src-homology domain (SH2).

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., systemically or locally by direct injection at a tissue site such as bone marrow), or generated in situ such that they hybridize with or bind to cellular mRNA and/or genomic DNA encoding the SHIP to thereby inhibit expression of the SHIP gene. Alternatively, antisense nucleic acid molecules can be modified to target selected cells (such as hematopoietic stem cells and/or hematopoietic progenitor cells) and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or poi III promoter can be used.

In yet another embodiment, the antisense oligonucleotide of the invention is an alpha-anomeric nucleic acid molecule. An alpha-anomeric nucleic acid molecule forms specific double-stranded hybrids with complementary RNA in which, contrary to the usual beta-units, the strands run parallel to each other (Gaultier et al., 1987). The antisense nucleic acid molecule can also comprise a 2′-o-methylribonucleotide (Inoue et al., 1987b) or a chimeric RNA-DNA analogue (Inoue et al., 1987a).

SHIP expression can be inhibited by targeting nucleotide sequences complementary to the regulatory region of the SHIP gene to form triple helical structures that prevent expression of SHIP in target cells. See generally, Helene, (1991); Helene (1992); and Maher (1992). The potential sequences that can be targeted for triple helix formation can be increased by creating a so called “switchback” nucleic acid molecule. Switchback molecules are synthesized in an alternating 5°-3′, 3′-5′ manner, such that they base pair with first one strand of a duplex and then the other, eliminating the necessity for a sizeable stretch of either purines or pyrimidines to be present on one strand of a duplex.

### Ribozymes

Ribozymes are a type of RNA that can be engineered to enzymatically cleave and inactivate other RNA targets in a specific, sequence-dependent fashion. Ribozymes and methods for their delivery are well known in the art (Hendry et al., 2004; Grassi et al., 2004; Bagheri et al., 2004; Kashani-Sabet, 2004), each of which are incorporated herein by reference in its entirety. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for SHIP mRNA can include one or more sequences complementary to a nucleotide sequence within the SHIP mRNA, and a sequence having a known catalytic sequence responsible for mRNA cleavage (see U.S. Pat. No. 5,093,246 or Haselhoff and Gerlach, 1988). For example, a derivative of a Tetrahymena L-19 IVS RNA can be constructed in which the nucleotide sequence of the active site is complementary to the nucleotide sequence to be cleaved in the mRNA encoded by a uORF of an extended, overlapping 5′-UTR AS mRNA species (see, e.g., Cech et al. U.S. Pat. No. 4,987,071; and Cech et al. U.S. Pat. No. 5,116,742). Alternatively, SHIP mRNA can be used to select a catalytic RNA having a specific ribonuclease activity from a pool of RNA molecules (see, e.g., Bartel and Szostak, 1993).

### Nucleic Acid Targets

The nucleic acid targets of SHIP inhibitors that are polynucleotides (referred to herein as “polynucleotide SHIP inhibitors” or “nucleic acid SHIP inhibitors”) such as the antisense, interfering RNA molecules, and ribozymes described herein, may be any portion of the SHIP gene or SHIP mRNA. In some embodiments, the nucleic acid target is the SHIP enzymatic domain (inositol 5′-phosphatase) or the amino-terminal src-homology domain (SH2). In other embodiments, the nucleic acid target is a translation initiation site, 3′ untranslated region, or 5′ untranslated region

As indicated above, the nucleotide sequences of mouse SHIP and human SHIP have been publicly available for several years (GenBank Accession Numbers NM_{—}10566 and NM_{—}005541, respectively, on the NCBI database). Pair-wise alignment scoring of orthologues shows high levels of homology among SHIP sequences of humans, mice, and rats. Each sequence has the SHIP enzymatic domain (inositol 5′-phosphatase domain), and the degree of nucleotide homology between human, mouse, and rat is over 85%. Furthermore, mice and humans are believed to have the same five SHIP protein isofolins. In a preferred embodiment, the polynucleotide SHIP inhibitor (e.g., interfering RNA, antisense oligonucleotide, ribozyme) targets an mRNA sequence shared by all known hematopoietic SHIP isoforms in humans. Such target sequence can be readily determined by those skilled in the art due to the extensive amount of sequence overlap between the isoforms.

The target SHIP sequence can be within any orthologue of SHIP, such as sequences substantially identical to human, mouse, rat, or bovine, or a portion of any of the foregoing, including but not limited to GenBank Accession Numbers NM_{—}005541 and NM_{—}001017915 (human), NM_{—}10566 (mouse), and U55192 (rat).

Table 1 lists thirteen target sequences predicted to have good specificity and knockdown potential against the human SHIP cDNA sequence.

TABLE 1

| | | | | |
|---|---|---|---|---|
| Seq. Identifier | Target Sequence | Region | Start (nt) | GC Content |

| | | | | |
|---|---|---|---|---|
| SEQ ID NO: 3 | GCCTGTTGTCATCCATTGA | ORF | 890 | 47.37% |
| SEQ ID NO: 4 | ATAAGTTGGTGATCTTGGT | ORF | 1145 | 36.84% |
| SEQ ID NO: 5 | GCCACATCTGTACTGACAA | ORF | 1589 | 47.37% |
| SEQ ID NO: 6 | AGACAGGCATTGCAAACAC | ORF | 1613 | 47.37% |
| SEQ ID NO: 7 | ACATCACTCACCGCTTCAC | ORF | 1802 | 52.63% |
| SEQ ID NO: 8 | TCTTAACTACCGTGTGGAT | ORF | 1842 | 42.11% |
| SEQ ID NO: 9 | AATACGCCTACACCAAGCA | ORF | 2039 | 47.37% |
| SEQ ID NO: 10 | GTACCAGCGACATCATGAC | ORF | 2156 | 52.63% |
| SEQ ID NO: 11 | GCGACATCATGACGAGTGA | ORF | 2162 | 52.63% |
| SEQ ID NO: 12 | AGGACAGATTGAGTTTCTC | ORF | 2265 | 42.11% |
| SEQ ID NO: 13 | GGTGCTATGCCACATTGAA | ORF | 2285 | 47.37% |
| SEQ ID NO: 14 | GTTTGGTGAGACTCTTCCA | ORF | 2418 | 47.37% |
| SEQ ID NO: 15 | AGACGGAGCGTGATGAATC | ORF | 2687 | 52.63% |

The term “orthologue” as used herein refers to a sequence which is substantially identical to a reference sequence. The term “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that contain a common structural domain having at least about 60%, or 65% identity, likely 75% identity, more likely 85%, 90%, 91%, 92%, 93%, 94%, 95%, 96%, 97%, 98% or 99% identity are defined herein as substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 50%, at least 60%, at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (1970) algorithm, which has been incorporated into the GAP program in the GCG software package (available at the official Accelrys web site), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at the official Accelrys web site), using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other orthologs, e.g., family members or related sequences. Such searches can be performed using the NBLAST and XBLAST programs (version 2.0) of Altschul et al., 1990). BLAST nucleotide searches can be performed with the NBLAST program, score=100, word length=12, to obtain nucleotide sequences homologous to known SHIP nucleic acid sequences. BLAST protein searches can be performed with the XBLAST program, score=50, word length=3, to obtain amino acid sequences homologous to known polypeptide products of the SHIP gene. To obtain gapped alignments for comparison purposes, Gapped BLAST can be utilized as described in Altschul et al., 1997. When utilizing BLAST and Gapped BLAST programs, the default parameters of the respective programs (e.g., XBLAST and NBLAST) can be used (see the National Center for Biotechnology Information web site of the National Institutes of Health).

Orthologs can also be identified using any other routine method known in the art, such as screening a cDNA library, e.g., a human cDNA library, using a probe designed to identify sequences which are substantially identical to a reference sequence.

### Pharmaceutical Compositions and Methods of Administration

The SHIP inhibitors of the subject invention (such as small molecules, interfering RNA molecules, antisense molecules, and ribozymes) can be incorporated into pharmaceutical compositions. Such compositions typically include the SHIP inhibitor and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Formulations (compositions) are described in a number of sources that are well known and readily available to those skilled in the art. For example, Remington's Pharmaceutical Sciences (Martin E. W., Easton Pa., Mack Publishing Company, 19^{th} ed., 1995) describes formulations which can be used in connection with the subject invention.

A pharmaceutical composition comprising a SHIP inhibitor is formulated to be compatible with its intended route of administration. Examples of routes of administration include parenteral, e.g., intravenous, intradermal, subcutaneous, oral (e.g., inhalation), topical, transdermal, transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL (BASF, Parsippany, N.J.) or phosphate buffered saline (PBS). In all cases, the composition must be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can also be included in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polynucleotide of the invention) in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the SHIP inhibitors can be delivered in the form of an aerosol spray from a pressurized container or dispenser which contains a suitable propellant, e.g., a gas such as carbon dioxide, or a nebulizer. Such inhalation methods and inhalant formulations include those described in U.S. Pat. No. 6,468,798.

Systemic administration of SHIP inhibitors can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compound (e.g., polynucleotides of the invention) are formulated into ointments, salves, gels, or creams, as generally known in the art.

The pharmaceutical compositions can also be prepared in the form of suppositories (e.g., with conventional suppository bases such as cocoa butter and other glycerides) or retention enemas for rectal delivery.

The SHIP inhibitors can also be administered by transfection or infection using methods known in the art, including but not limited to the methods described in McCaffrey et al., 2002 (hydrodynamic transfection); Xia et al., 2002 (viral-mediated delivery); or Putnam, 1996.

SHIP inhibitors that are polynucleotides can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in Hamajima et al., 1998. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the polynucleotide SHIP inhibitors are prepared with carriers that will protect the polynucleotide against rapid elimination from, or degradation in, the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions (including liposomes targeted to antigen-presenting cells with monoclonal antibodies) can also be used as pharmaceutically acceptable carriers. Strategies that inhibit members of the RNAse A family of enzymes or can otherwise protect polynucleotide SHIP inhibitors from these enzymes may be utilized. For example, U.S. Pat. No. 6,096,720 (Love et al.) describes oligonucleotides targeted to human raf mRNA, which are entrapped in sterically stabilized liposomes. In one embodiment, the oligonucleotide in Love et al. is a chimeric olignonucleotide containing a first region to enhance target affinity and a second region that is a substrate for RNase. siSHIELD RNAse inhibitor is designed to prevent degradation of siRNA by RNase (MP BIOMEDICALS, Irvine, Calif.). A strategy for the compaction of short oligonucleotides into well-defined condensates may also be used to deliver the polynucleotides of the subject invention (Sarkar T. et al., 2005), which is incorporated herein by reference in its entirety.

In particular, suitable techniques for cellular administration of the polynucleotide SHIP inhibitors, such as interfering RNA, in vivo are disclosed in the following articles:

### General Reviews:

Borkhardt, 2002; Hannon, 2002; McManus and Sharp, 2002; Scherr et al., 2003b; Shuey et al., 2002; Gilmore et al., 2004; Dykxhoorn and Lieberman, 2005.

### Systemic Delivery Using Liposomes:

Lewis et al., 2002; Paul et al., 2002; Song et al., 2003; Sorensen et al., 2003.

### Virus Mediated Transfer:

Abbas-Terki et al., 2002; Barton and Medzhitov, 2002; Devroe and Silver, 2002; Lori et al., 2002; Matta et al., 2003; Qin et al., 2003; Scherr, M. et al., 2003a; Shen et al., 2003; Lee et al., 2005.

### Peptide Delivery:

Morris et al., 2000; Simeoni et al., 2003.

Song E. et al. describe antibody mediated in vivo delivery of siRNAs via cell-surface receptors (Song et al., 2005). This cell-specific delivery technique can be used to target interfering RNA molecules to the cell-surface markers on cells such HSC (CD34, CD59, Thy1).

Other technologies that may be suitable for delivery of polynucleotide SHIP inhibitors, such as interfering RNA, to the target cells are based on nanoparticles or nanocapsules such as those described in U.S. Pat. No. 6,649,192B and 5,843,509B. Recent technologies that may be employed for selecting, delivering, and monitoring interfering RNA molecules include Raab and Stephanopoulos, 2004; Huppi et al., 2005; Spagnou et al., 2004; Muratovska and Eccles, 2004; Kumar et al., 2003; Chen et al., 2005; Dykxhoorn et al., 2006; Rodriguez-Lebron and Paulson, 2005; Pai et al., 2005; Raoul et al., 2005; Manfredsson et al., 2005; Downward, 2004.

A mixture of SHIP inhibitors, of the same type or different types, may be introduced into cells in vivo. For example, a mixture or pool of polynucleotide SHIP inhibitors such as interfering RNA molecules (e.g., 2-4 interfering molecules or more) can be introduced into cells (Oleinikov et al., 2005). Preferably, the interfering RNA molecules target different regions of the SHIP mRNA. Preferably, the interfering RNA molecules have been previously validated as individually functioning to reduce SHIP expression. The individual interfering RNAs of the mixture can be chemically synthesized (Elbashir S. M. et al., 2001b) or introduced as short DNA templates containing RNA polymerase promoter, which are transcribed within the cells in vitro or in vivo (Yu et al., 2002).

Toxicity and therapeutic efficacy of compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions which exhibit high therapeutic indices can be used. While compositions that exhibit toxic side effects can be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosage for use in humans. The dosage of such compositions generally lies within a range of circulating concentrations that include the ED50 with little or no toxicity. The dosage can vary within this range depending upon the dosage form employed and the route of administration utilized. For any composition used in the method of the invention, the therapeutically effective dose can be estimated initially from cell culture assays. A dose can be formulated in animal models to achieve a circulating plasma concentration range that includes the IC50 (i.e., the concentration of the test composition which achieves a half-maximal inhibition of symptoms) as determined in cell culture. Such information can be used to more accurately determine useful doses in humans. Levels in plasma can be measured, for example, by high performance liquid chromatography.

The SHIP inhibitor can be administered on any appropriate schedule, e.g., from one or more times per day to one or more times per week; including once every other day, for any number of days or weeks, e.g., 1 day, 2 days, 3 days, 4 days, 5 days, 6 days, 1 week, 10 days, 2 weeks, 3 weeks, 4 weeks, 5 weeks, 6 weeks, 7 weeks, 8 weeks, 2 months, 3 months, 6 months, or more, or any variation thereon. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a SHIP inhibitor can include a single treatment or can include a series of treatments.

The polynucleotide SHIP inhibitors (e.g., interfering RNA, antisense oligonucleotide, or ribozyme) can be introduced (administered) into cells (such as mammalian cells) in vitro or in vivo using known techniques, as those described herein, to suppress gene expression. Similarly, genetic constructs (e.g., transcription vectors) containing DNA of the invention may be introduced into cells in vitro or in vivo using known techniques, as described herein, for transient or stable expression of RNA, to suppress gene expression. When administered to the cells in vivo, the polynucleotide SHIP inhibitors can be administered to a subject systemically (e.g., intravenously), for example, or administered locally at the site of the cells (such as the peripheral blood, bone marrow, or spleen).

The cells in which the polynucleotide SHIP inhibitors are introduced may be any cell containing SHIP mRNA. Preferably, the target cells are bone marrow cells, such as hematopoietic cells. More preferably, the target cells are HSC and/or HPC. The cells can be primary cells, cultured cells, cells of cell lines, etc. In one embodiment, the cells are from bone marrow.

Mammalian species which benefit from the disclosed methods include, but are not limited to, primates, such as humans, apes, chimpanzees, orangutans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. As used herein, the terms “subject”, “patient”, and “individual” are used interchangeably and intended to include such human and non-human mammalian species. Host cells comprising exogenous polynucleotides of the invention may be administered to the subject, and may, for example, be autogenic (use of one's own cells), allogenic (from one person to another), or transgenic or xenogenic (from one mammalian species to another mammalian species), relative to the subject.

The polynucleotide SHIP inhibitors of the invention can be inserted into genetic constructs, e.g., viral vectors, retroviral vectors, expression cassettes, or plasmid viral vectors, e.g., using methods known in the art, including but not limited to those described in Xia et al., (2002). Genetic constructs can be delivered to a subject by, for example, inhalation, orally, intravenous injection, local administration (see U.S. Pat. No. 5,328,470) or by stereotactic injection (see, e.g., Chen et al., 1994). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the polynucleotide delivery system.

The polynucleotide SHIP inhibitors can be small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides (Brummelkamp et al., 2002; Lee et al., 2002; Miyagishi and Taira, 2002; Paddison et al., 2002; Paul, 2002; Sui, 2002; Yu et al., 2002.

SiRNAs targeting SHIP mRNA may be fused to other nucleotide molecules, or to polypeptides, in order to direct their delivery or to accomplish other functions. Thus, for example, fusion proteins comprising a siRNA oligonucleotide that is capable of specifically interfering with expression of SHIP may comprise affinity tag polypeptide sequences, which refers to polypeptides or peptides that facilitate detection and isolation of the polypeptide via a specific affinity interaction with a ligand. The ligand may be any molecule, receptor, counter-receptor, antibody or the like with which the affinity tag may interact through a specific binding interaction as provided herein. Such peptides include, for example, poly-His or “FLAG” or the like, e.g., the antigenic identification peptides described in U.S. Pat. No. 5,011,912 and in Hopp et al., (1988), or the XPRESS epitope tag (INVITROGEN, Carlsbad, Calif.). The affinity sequence may be a hexa-histidine tag as supplied, for example, by a pBAD/His (INVITROGEN) or a pQE-9 vector to provide for purification of the mature polypeptide fused to the marker in the case of a bacterial host, or, for example, the affinity sequence may be a hemagglutinin (HA) tag when a mammalian host, e.g., COS-7 cells, is used. The HA tag corresponds to an antibody defined epitope derived from the influenza hemagglutinin protein (Wilson et al., 1984).

The present invention also relates to the administration of vectors and constructs that include or encode polynucleotide SHIP inhibitors (e.g., siRNA), and in particular to “recombinant nucleic acid constructs” that include any nucleic acid such as a DNA polynucleotide segment that may be transcribed to yield SHIP-specific siRNA polynucleotides according to the invention as provided above; to host cells which are genetically engineered with vectors and/or constructs of the invention and to the production of siRNA polynucleotides, polypeptides, and/or fusion proteins of the invention, or fragments or variants thereof, by recombinant techniques. siRNA sequences disclosed herein as RNA polynucleotides may be engineered to produce corresponding DNA sequences using well-established methodologies such as those described herein. Thus, for example, a DNA polynucleotide may be generated from any siRNA sequence described herein, such that the present siRNA sequences will be recognized as also providing corresponding DNA polynucleotides (and their complements). These DNA polynucleotides are therefore encompassed within the contemplated invention, for example, to be incorporated into the subject invention recombinant nucleic acid constructs from which siRNA may be transcribed.

According to the present invention, a vector may comprise a recombinant nucleic acid construct containing one or more promoters for transcription of an RNA molecule, for example, the human U6 snRNA promoter (see, e.g., Miyagishi et al., 2002; Lee et al., 2002; Paul et al., 2002; Grabarek et al., 2003; see also Sui et al., 2002). Each strand of a siRNA polynucleotide may be transcribed separately each under the direction of a separate promoter and then may hybridize within the cell to form the siRNA polynucleotide duplex. Each strand may also be transcribed from separate vectors (see Lee et al., 2002). Alternatively, the sense and antisense sequences specific for a SHIP mRNA sequence may be transcribed under the control of a single promoter such that the siRNA polynucleotide forms a hairpin molecule (Paul et al., 2002). In such instance, the complementary strands of the siRNA specific sequences are separated by a spacer that comprises at least four nucleotides, but may comprise at least 5, 6, 7, 8, 9, 10, 11, 12, 14, 16, or 18 or more nucleotides as described herein. In addition, siRNAs transcribed under the control of a U6 promoter that form a hairpin may have a stretch of about four uridines at the 3′ end that act as the transcription termination signal (Miyagishi et al., 2002; Paul et al., 2002). By way of illustration, if the target sequence is 19 nucleotides, the siRNA hairpin polynucleotide (beginning at the 5′ end) has a 19-nucleotide sense sequence followed by a spacer (which as two uridine nucleotides adjacent to the 3′ end of the 19-nucleotide sense sequence), and the spacer is linked to a 19 nucleotide antisense sequence followed by a 4-uridine terminator sequence, which results in an overhang. siRNA polynucleotides with such overhangs effectively interfere with expression of the target polypeptide. A recombinant construct may also be prepared using another RNA polymerase III promoter, the H1 RNA promoter, that may be operatively linked to siRNA polynucleotide specific sequences, which may be used for transcription of hairpin structures comprising the siRNA specific sequences or separate transcription of each strand of a siRNA duplex polynucleotide (see, e.g., Brummelkamp et al., 2002; Paddison et al., 2002). DNA vectors useful for insertion of sequences for transcription of an siRNA polynucleotide include pSUPER vector (see, e.g., Brummelkamp et al., 2002); pAV vectors derived from pCWRSVN (see, e.g., Paul et al., 2002); and pIND (see, e.g., Lee et al., 2002), or the like.

Polynucleotide SHIP inhibitors can be expressed in mammalian cells, yeast, bacteria, or other cells under the control of appropriate promoters, providing ready systems for evaluation of siRNA polynucleotides that are capable of interfering with SHIP expression as provided herein. Appropriate cloning and expression vectors for use with prokaryotic and eukaryotic hosts are described, for example, by Sambrook, et al., Molecular Cloning: A Laboratory Manual, Third Edition, Cold Spring Harbor, N.Y., (2001).

The appropriate DNA sequence(s) may be inserted into the vector by a variety of procedures. In general, the DNA sequence is inserted into an appropriate restriction endonuclease site(s) by procedures known in the art. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described, for example, in Ausubel et al. (1993 Current Protocols in Molecular Biology, Greene Publ. Assoc. Inc. & John. Wiley & Sons, Inc., Boston, Mass.); Sambrook et al. (2001 Molecular Cloning, Third Ed., Cold Spring Harbor Laboratory, Plainview, N.Y.); Maniatis et al. (1982 Molecular Cloning, Cold Spring Harbor Laboratory, Plainview, N.Y.); and elsewhere.

The DNA sequence in the expression vector is operatively linked to at least one appropriate expression control sequence (e.g., a promoter or a regulated promoter) to direct mRNA synthesis. Representative examples of such expression control sequences include LTR or SV40 promoter, the E. coli lac or trp, the phage lambda P_{L} promoter and other promoters known to control expression of genes in prokaryotic or eukaryotic cells or their viruses. Promoter regions can be selected from any desired gene using CAT (chloramphenicol transferase) vectors or other vectors with selectable markers. Examples of Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art, and preparation of certain particularly preferred recombinant expression constructs comprising at least one promoter or regulated promoter operably linked to a polynucleotide of the invention is described herein.

As noted above, in certain embodiments the vector may be a viral vector such as a mammalian viral vector (e.g., retrovirus, adenovirus, adeno-associated virus, lentivirus). For example, retroviruses from which the retroviral plasmid vectors may be derived include, but are not limited to, Moloney Murine Leukemia Virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus.

The viral vector includes one or more promoters. Suitable promoters which may be employed include, but are not limited to, the retroviral LTR; the SV40 promoter; and the human cytomegalovirus (CMV) promoter described in Miller, et al. (1989), or any other promoter (e.g., cellular promoters such as eukaryotic cellular promoters including, but not limited to, the histone, pol III, and beta-actin promoters). Other viral promoters that may be employed include, but are not limited to, adenovirus promoters, adeno-associated virus promoters, thymidine kinase (TK) promoters, and B19 parvovirus promoters. The selection of a suitable promoter will be apparent to those skilled in the art from the teachings contained herein, and may be from among either regulated promoters (e.g., tissue-specific or inducible promoters) or promoters as described above. A tissue-specific promoter allows preferential expression of the polynucleotide SHIP inhibitor in a given target tissue, thereby avoiding expression in other tissues. For example, to target genes specifically in the heart, a number of cardiac-specific regulatory elements can be used. An example of a cardiac-specific promoter is the ventricular form of MLC-2v promoter (see, Zhu et al., 1993; Navankasattusas et al., 1992) or a variant thereof such as a 281 by fragment of the native MLC-2v promoter (nucleotides −264 to +17, GenBank Accession No. U26708). Examples of other cardiac-specific promoters include alpha myosin heavy chain (Minamino et al., 2001) and myosin light chain-2 (Franz et al., 1993). Endothelial cell gene promoters include endoglin and ICAM-2. See Velasco et al., 2001. Liver-specific promoters include the human phenylalanine hydroxylase (PAH) gene promoters (Bristeau et al., 2001), hB1F (Zhang et al., 2001), and the human C-reactive protein (CRP) gene promoter (Ruther et al., 1993). Promoters that are kidney-specific include CLCN5 (Tanaka et al., 1999), renin (Sinn et al., 2000), androgen-regulated protein, sodium-phosphate cotransporter, renal cytochrome P-450, parathyroid hormone receptor and kidney-specific cadherin. See El-Meanawy et al., (2000). An example of a pancreas-specific promoter is the pancreas duodenum homeobox 1 (PDX-1) promoter (Samara et al., 2002). A number of brain-specific promoters may be useful in the invention and include the thy-1 antigen and gamma-enolase promoters (Vibert et al., 1989), the glial-specific glial fibrillary acidic protein (GFAP) gene promoter (Cortez et al., 2000), and the human FGF1 gene promoter (Chiu et al., 2000). The GATA family of transcription factors have promoters directing neuronal and thymocyte-specific expression (see Asnagli et al., 2002).

In another aspect, the present invention relates to the administration of host cells containing the above described recombinant constructs. Host cells are genetically engineered/modified (transduced, transformed or transfected) with the vectors and/or expression constructs of this invention that may be, for example, a cloning vector, a shuttle vector, or an expression construct. The vector or construct may be, for example, in the form of a plasmid, a viral particle, a phage, etc. The engineered host cells can be cultured in conventional nutrient media modified as appropriate for activating promoters, selecting transformants or amplifying particular genes such as genes encoding siRNA polynucleotides or fusion proteins thereof. The culture conditions for particular host cells selected for expression, such as temperature, pH and the like, will be readily apparent to the ordinarily skilled artisan.

The host cell can be a higher eukaryotic cell, such as a mammalian cell, or a lower eukaryotic cell, such as a yeast cell, or the host cell can be a prokaryotic cell, such as a bacterial cell. Representative examples of appropriate host cells according to the present invention include, but need not be limited to, bacterial cells, such as E. coli, Streptomyces, Salmonella typhimurium; fungal cells, such as yeast; insect cells, such as Drosophila S2 and Spodoptera Sf9; animal cells, such as CHO, COS or 293 cells; adenoviruses; plant cells, or any suitable cell already adapted to in vitro propagation or so established de novo.

Various mammalian cell culture systems can also be employed to produce polynucleotide SHIP inhibitors from recombinant nucleic acid constructs of the present invention. The invention is therefore directed in part to a method of producing a polynucleotide, such as an siRNA, by culturing a host cell comprising a recombinant nucleic acid construct that comprises at least one promoter operably linked to a polynucleotide SHIP inhibitor. In certain embodiments, the promoter may be a regulated promoter as provided herein, for example a tetracycline-repressible promoter. In certain embodiments the recombinant expression construct is a recombinant viral expression construct as provided herein. Examples of mammalian expression systems include the COS-7 lines of monkey kidney fibroblasts, described by Gluzman, (1981), and other cell lines capable of expressing a compatible vector, for example, the C127, 3T3, CHO, HeLa, HEK, and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences, for example as described herein regarding the preparation of recombinant polynucleotide constructs. DNA sequences derived from the SV40 splice, and polyadenylation sites may be used to provide the required nontranscribed genetic elements. Introduction of the construct into the host cell can be effected by a variety of methods with which those skilled in the art will be familiar, including but not limited to, for example, liposomes including cationic liposomes, calcium phosphate transfection, DEAE-Dextran mediated transfection, or electroporation (Davis et al., 1986), or other suitable technique.

The expressed polynucleotides may be useful in intact host cells; in intact organelles such as cell membranes, intracellular vesicles or other cellular organelles; or in disrupted cell preparations including but not limited to cell homogenates or lysates, microsomes, uni- and multilamellar membrane vesicles or other preparations. Alternatively, expressed polynucleotides can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Finally, high performance liquid chromatography (HPLC) can be employed for final purification steps.

As used herein, the terms “administer”, “introduce”, “apply”, “treat”, “transplant”, “implant”, “deliver”, and grammatical variations thereof, are used interchangeably to provide SHIP inhibitors to target cells in vitro (e.g., ex vivo) or in vivo, or provide genetically modified (engineered) cells of the subject invention to a subject.

As used herein, the term “co-administration” and variations thereof refers to the administration of two or more agents simultaneously (in one or more preparations), or consecutively. For example, one or more types of genetically modified cells of the invention can be co-administered with other agents.

As used in this specification, including the appended claims, the singular “a”, “an”, and “the” include plural reference unless the contact dictates otherwise. Thus, for example, a reference to “a polynucleotide” includes more than one such polynucleotide. A reference to “a nucleic acid sequence” includes more than one such sequence. A reference to “a cell” includes more than one such cell.

As used herein, the term “or” is inclusive of “both” (i.e., and/or). For example, as used herein, reference to hematopoietic stem cells (HSC) “or” hematopoietic progenitors (HPC) includes “either” or “and” (i.e., and/or).

The terms “comprising”, “consisting of” and “consisting essentially of” are defined according to their standard meaning. The terms may be substituted for one another throughout the instant application in order to attach the specific meaning associated with each term.

In general, the target nucleic acid is DNA or RNA. However, inventive methods may employ, for example, samples that contain DNA, or DNA and RNA, including messenger RNA, wherein DNA or RNA may be single stranded or double stranded, or a DNA-RNA hybrid may be included in the sample. A mixture of nucleic acids may also be employed. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. The nucleic acid-containing sample used for determination of the sensitivity of the target cells to radiation therapy may be extracted by a variety of techniques such as that described by Sambrook, et al. (Molecular Cloning: A Laboratory Manual, Cold Spring Harbor, N.Y., 1989; incorporated in its entirety herein by reference).

Cells expressing the target nucleic acid isolated from a subject can be obtained in a biological specimen from the subject. The cells, or nucleic acid, can be isolated from tumor tissue, blood, plasma, serum, lymph, lymph nodes, spleen, bone marrow, or any other biological specimen containing the target nucleic acid. Tumor tissue, blood, plasma, serum, lymph, spleen, and bone marrow are obtained by various medical procedures known to those of skill in the art.

The inventive methods are useful for producing a clinical response in treatment of various disorders associated with bone marrow function. A cell proliferative disorder as described herein may be a neoplasm. Such neoplasms are either benign or malignant. The term “neoplasm” refers to a new, abnormal growth of cells or a growth of abnormal cells that reproduce faster than normal. A neoplasm creates an unstructured mass (a tumor) which can be either benign or malignant. The term “benign” refers to a tumor that is noncancerous, e.g., its cells do not invade surrounding tissues or metastasize to distant sites. The term “malignant” refers to a tumor that is metastatic, invades contiguous tissue or no longer under normal cellular growth control.

As used herein, “a clinical response” includes the response of a subject to modulation of the gene of interest. Criteria for determining a response to therapy are widely accepted and enable comparisons of the efficacy alternative treatments (see Slapak and Kufe, Principles of Cancer Therapy, in Harrisons's Principles of Internal Medicine, 13^{th} edition, eds. Isselbacher et al., McGraw-Hill, Inc. 1994). A complete response (or complete remission) is the disappearance of all detectable malignant disease. A partial response is an approximately 50 percent decrease in the product of the greatest perpendicular diameters of one or more lesions. There can be no increase in size of any lesion or the appearance of new lesions. Progressive disease means at least an approximately 25 percent increase in the product of the greatest perpendicular diameter of one lesion or the appearance of new lesions. The response to treatment is evaluated after the subjects have completed therapy.

SHIP inhibitors (also referred to herein as “active compounds”) can be incorporated into pharmaceutical compositions suitable for administration. Such compositions typically comprise the SHIP-inhibiting small molecule, nucleic acid molecule, protein, or antibody and a pharmaceutically acceptable carrier. As used herein the language “pharmaceutically acceptable carrier” is intended to include any and all solvents, dispersion media, coatings, antibacterial and anti fungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to target cells with monoclonal antibodies) can also be used as pharmaceutically acceptable carriers. These can be prepared according to methods known to those skilled in the art, for example, as described in U.S. Pat. No. 4,522,811.

It is also advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. The pharmaceutical compositions can be included in a container, pack, or dispenser together with instructions for administration.

All patents, patent applications, provisional applications, and publications referred to or cited herein, supra or infra, are incorporated by reference in their entirety, including all figures and tables, to the extent they are not inconsistent with the explicit teachings of this specification.

### Materials and Methods

Mice. The production of SHIP^{-/-} and MxCreSHIP^{flox/flox} mice has been previously described (Wang et al., 2002; Paraiso et al., 2007). In brief, SHIP^{-/-} mice were generated by deletion of the promoter and first exon of SHIP via a Cre-LoxP strategy and backcrossed to C57BL6/J background. MxCreSHIP^{flox/flox} mice were generated using SHIP^{flox/flox} and MxCre mice (Jackson Laboratory, Bar Harbor, Me.). MxCreSHIP^{flox/flox}:WT-Ly5.1 and SHIP^{flox/flox}:WT-Ly5.1 BM chimeras were created by co-transplanting 5×10⁵ WBM cells from MxCreSHIP^{flox/flox}(CD45.2⁺) or SHIP^{flox/flox}(CD45.2⁺) plus 5×10⁵ cells from WT-Ly5.1(CD45.1⁺) mice into lethally irradiated CD45.1⁺45.2⁺ recipients. All animal experiments were conducted with approval of the Univ. of South Florida IACUC.

Cell isolation. Bone marrow (BM) cells were flushed from femurs and tibias and collected in tissue media (TM) consisting of RPMI, 3% Fetal Bovine Serum (FBS), and 10 mM HEPES (Invitrogen, Carlsbad, Calif.). Cells were filtered through a 70 mm strainer (BD Bioscience, San Jose, Calif.) and red blood cell (RBC) lysis was performed at room temperature for 5 minutes. Cells were centrifuged and resuspended in staining media (SM) composed of Dulbecco phosphate-buffered saline (D-PBS), 3% FBS, and 10 mM HEPES. WBM used for transplantation was not RBC lysed, but simply flushed, washed and resuspended. Spleens were crushed with a 10 ml syringe plunger. Splenocytes were then treated like BM cells. Peripheral blood (PB) was obtained by sub-mandibular bleeding and collected in microtainers with K₂EDTA (BD, Franklin Lakes, N.J.). RBC lysis was performed twice to obtain PB mononuclear cells (PBMC).

Conditional deletion of SHIP. MxCreSHIP^{flox/flox}(CD45.2⁺) mice and MxCreSHIP^{flox/flox}:WT-Ly5.1 BM chimeras were conditionally ablated for SHIP expression through i.p. injection of polyinosinic-polycytidylic acid (polyI:C) (Sigma-Aldrich, St. Louis, Mo.). Mice were injected three times with 625 μg of polyI:C on days 1, 4, and 7.

Assessment of donor reconstitution in PBMC. All antibodies were from BD Biosciences (San Jose, Calif.), eBioscience (San Diego, Calif.) or CalTag/Invitrogen (Carlsbad, Calif.). PBMC were treated with CD16/CD32 mouse Fc block(2.4G2) on ice for 20 minutes then stained with a panel containing: CD45.1-PE(A20), CD45.2-FITC(104), B220-AlexaFluor700(RA3-6B2), CD3-PECy7(145-2C11), Mac1-APC(M1/70) and Gr1-APC(RB6-8C5). After 20-minutes on ice the cells were washed and resuspended in SM containing 75 ng/ml of 4′,6-diamidino-2-phenylindole dihydrochloride (DAPI) (Sigma, Saint Louis, Mo.) for dead cell exclusion. All flow cytometry acquisition was done on a FACS LSRII (BD Biosciences) and data was analyzed using FLOWJO software (Tree Star, Inc. Ashland, Oreg.).

In Situ SHIP deletion BM transplants. 5×10⁵ MxCreSHIP^{flox/flox}(CD45.2⁺) or SHIP^{flox/flox}(CD45.2⁺) WBM cells were co-transplanted (i.v.) with 5×10⁵ WBM competitor cells from WT-Ly5.1(CD45.1⁺) mice into lethally irradiated CD45.1⁺45.2⁺ recipients. The recipients were placed on antibiotic water prior to receiving a split dose of 11-Gy at least 2-hours before transplantation. After 60 days recipient mice were treated with the polyI:C series.

Secondary BM transplants from In Situ SHIP Deleted Mice. Five months after polyI:C treatment of MxCreSHIP^{flox/flox}:WT-Ly5.1 and SHIP^{flox/flox}:WT-Ly5.1 BM chimeras, the mice were sacrificed and 1×10⁶ WBM cells were transplanted i.v. into lethally irradiated CD45.1⁺45.2⁺ recipients.

Systemic SHIP Deletion BM Transplants.

MxCreSHIP^{flox/flox}(CD45.2⁺) mice and SHIP^{flox/flox}(CD45.2⁺) mice were pre-treated with the polyI:C series and SHIP deletion was confirmed by Western blot of PBMC. 5×10⁵ WBM cells from. MxCreSHIP^{flox/flox}(CD45.2⁺) or SHIP^{flox/flox}(CD45.2⁺) mice were co-transplanted (i.v.) with 5×10⁵ WBM competitors from WT-Ly5.1(CD45.1⁺) mice into lethally irradiated CD45.1⁺45.2⁺ recipients.

Western blot confirmation of SHIP deletion. Single positive CD45.1⁺ or CD45.2⁺ cells were isolated from splenocytes by cell sorting for Western blot of SHIP expression. Cell lysates were prepared using modified RIPA lysis buffer (Upstate Cell Signaling, Millipore, Billerica, Mass.). Lysate supernatants were loaded onto a 4-12% Bis-tris gel, run for 60 minutes at 200 volts, and transferred to a Hybond-ECL nitrocellulose membrane (Amersham Biosciences, Piscataway, N.J.). The membrane was blocked with Odyssey blocking buffer (LICOR Biosciences, Lincoln, Nebr.) and probed with 1 μg/ml P1C1 (Santa Cruz Biotechnology, Santa Cruz, Calif.) then anti-mouse IR-700 at 1:8,000 (Molecular Probes, Carlsbad, Calif.). SHIP protein was detected using Odyssey detection reagents and an infrared imager.

Assessment of PB multi-lineage reconstitution. At monthly intervals, the level of donor reconstitution was assessed. PB was collected and RBC lysed as previously described. PBMC were treated with CD16/CD32 mouse Fc block(2.4G2) then stained with a panel containing: CD45.1-PE(A20), CD45.2-PercpCy5.5(104), B220-AlexaFluor700(RA3-6B2), CD3-PECy7(145-2C11), Mac1-APCCy7(M1/70), Gr1-FITC(RB6-8C5), and NK1.1-APC(PK136); or CD45.1-PE(A20), CD45.2-PercpCy5.5(104), B220-AlexaFluor700(RA3-6B2), CD3-PECy7(145-2C11), Mac1-APCCy7(M1/70), Gr1-APCCy7(RB6-8C5), Ter119-APC(TER-119), and DAPI.

Assessment of BM multi-lineage reconstitution. At sacrifice, the level of donor BM reconstitution was assessed. BM was processed as previously described. BM was treated with CD16/CD32 mouse Fc block(2.4G2) then stained with: CD45.1-PE(A20), CD45.2-PercpCy5.5(104), B220-AlexaFluor700(RA3-6B2), CD3-PECy7(145-2C11), Mac1-APCCy7(M1/70), Gr1-FITC(RB6-8C5), and NK1.1-APC(PK136); or CD45.1-PE(A20), CD45.2-PercpCy5.5(104), Mac1-APCCy7(M1/70), Gr1-FITC(RB6-8C5), Ter119-APC(TER-119), and DAPI.

HSC compartment and CXCR4 expression analysis. BM cells were treated with CD16/CD32 mouse Fc block(2.4G2) then stained with markers for the Lineage^{-/low}c-Kit⁺Sca1⁺CD48⁻ (KLSCD48) phenotype (Kiel et al., 2005). The HSC stain included CD45.1-Purified(A20) conjugated to Pac Orange using Zenon technology, CD45.2-PerCPCy5.5(104), Sca1-PECy7(D7), c-Kit-APC-AF750(2B8), CD48-APC(HM48-1), CXCR4-PE(2B11/CXCR4), and a lineage (Lin) panel on FITC; CD2(RM2-5), CD3e(145-2C11), CD4(GK1.5), CD5(53-7.3), CD8a(53-6.7), B220(RA3-6B2), Gr-1(RB6-8C5), Mac-1(M1/70), NK1.1(PK136), and Ter119(TER-119). For some experiments the HSC stain included Sca1-FITC(E13-161.7), c-Kit-PECy7(2B8), CD48-APC(HM48-1), CXCR4-biotin(2B11/CXCR4), and a Lin panel on PE; CD2(RM2-5), CD3e(145-2C11), CD4(GK1.5), CD5(53-7.3), CD8a(53-6.7), B220(RA3-6B2), Gr-1(RB6-8C5), Mac-1(M1/70), NK1.1(PK136), and Ter119(TER-119). A secondary stain of Steptavidin-AF700 followed. Cells were resuspended in SM containing DAPI.

Calculation of Repopulation Units. Repopulating units (RU) were calculated as per Harrison et al. (1993): (C*CD45.2 repopulation(%))/(100−CD45.2 repopulation(%)), where 1C=10⁵ competitor cells. Since we used 5×10⁵ competitor cells, we used this equation:

RU=(5*CD45.2 repopulation(%))/(100−CD45.2 repopulation(%)). One RU is defined as the repopulating ability of 1×10⁵ untreated competitor BM cells (Harrison et al., 2003).

ELISA detection of cytokines in blood, BM plasma and cell culture supernatant. PB was collected, by sub-mandibular bleed or cardiac puncture, in 1.5 ml polypropylene tubes and left at RT for 4 hours to coagulate and then stored at 4° C. overnight. The following day, clots were removed and remaining blood was centrifuged at 4000 rpm for 10 minutes at 4° C. Serum was isolated by removing the supernatant to a new tube and storing it at −20° C. until later use or was sent for analysis by a service from Charles Rivers Laboratories Inc., where ELISA for multiple cytokines were performed. BM plasma was assayed for cytokines by flushing cleaned femurs with 5000 PBS. The PBS was then centrifuged at 1800 rpm for 5 minutes at 4° C. The supernatant was removed to a new tube and stored at −20° C. until later use. Cell culture supernatants were harvested at regular intervals and stored at −20° C. until later use. Stored supernatants were then assayed for stromal cell derived factor-1 (SDF-1) using the QUANTIKINE Immunoassay (R&D Systems, Minneapolis, Minn.).

Isolation and culture of stromal cells. Bone marrow was flushed from intact femurs and tibias into tissue culture flasks containing alpha-modified MEM with nucleotides (α-MEM) (Arnett et al. 1998). The α-MEM was supplemented with 15% FBS, 50 units penicillin-G/mL, 50 μg streptomycin/mL, 0.3 μg amphotericin-B/mL, and 50 μg ascorbate/mL. The flasks were kept at 37° C. with 5% CO₂. The cells were cultured undisturbed for three days, after which the media was changed and non-adherent cells removed.

Isolation and culture of osteoblasts. Cleaned and flushed femurs and tibias were scraped using a scalpel to remove remaining soft tissue (Bakker and Klein-Nulend, 2003). The bones were cut into 1-2 mm² pieces and incubated with shaking in a solution of 2 mg collagenase-II/mL in modified DMEM at 37° C. for 2 hours. Complete culture medium (cCM) was added to stop the collagenase action. The cCM consisted of DMEM (pH 7.4) supplemented with 2.2 g NaHCO₃/L, 10% FBS, 50 units penicillin-G/mL, 50 μg streptomycin/mL, 50 μg gentamycin/mL, 1.25 μg amphotericin-B/mL, and 100 μg ascorbate/mL. The bone pieces were washed three times with cCM and transferred to 25 cm² tissue culture flasks, which were kept at 37° C. with 5% CO₂.

Mesenchymal non-hematopoietic cell isolation. BM cells were collected and processed without RBC lysis. The cells were treated with CD16/CD32 mouse Fc block(2.4G2) then stained with: CD2-FITC(RM2-5), CD3e-FITC(145-2C11), CD4-FITC(GK1.5), CD5-FITC(53-7.3), CD8a-FITC(53-6.7), CD41-FITC(MWReg30), B220-FITC(RA3-6B2), Teri 19-FITC(TER-119), NK1.1-FITC(PK136), Mac1-FITC(M1/70), GR1-FITC(RB6-8C5), CD45-PE(30-F11), and DAPI. CD45⁻Lin⁻ cells were sorted using a FACS Aria cell sorter (BD Biosciences).

Western blots for SHIP and β-actin expression in BM niche. Cell lysates were prepared from cultured or sorted BM niche cells using a modified RIPA lysis buffer, then blotted as described above. The membrane was blocked with 5% non-fat milk in PBS with 0.1% Tween-20 (PBS-T) overnight at 4° C. and probed with 1 μg/ml P1C1 or 1 μg/ml Actin:C-11 (Santa Cruz Biotechnology) followed by anti-mouse IgG-HRP or anti-goat IgG-HRP at 1:1,000 (eBioscience). Protein was detected using SuperSignal West Femto Substrate (Thermo Scientific, Rockford, Ill.).

SHIP immunoprecipitation and Western blot for phosphotyrosine. Stromal cells cultured for 34 days were lysed in modified RIPA buffer. Lysate supernatants equivalent to 1×10⁶ cells each were pre-cleared with normal mouse IgG control and protein A/G-agarose beads (Santa Cruz Biotechnology). SHIP-1 was immunoprecipitated overnight with 0.2 μg P1C1 and protein A/G-agarose beads. The immunoprecipitated protein was blotted and the membrane blocked using the protocol above. It was then probed using an anti-phosphotyrosine, HRP-conjugated monoclonal antibody (4G10, Santa Cruz Biotechnology) diluted 1:1000 with 3% milk in PBS-T. Phosphorylated tyrosine was detected using SuperSignal West Femto Substrate diluted 1:5 with Pierce ECL Western Blotting Substrate (Thermo Scientific).

Statistical analysis. All statistical analyses were performed using PRISM (GraphPad, San Diego, Calif.).

### Example 1

### HSC Rendered SHIP-Deficient in a SHIP-Competent Niche Retain Multi-Lineage Repopulation

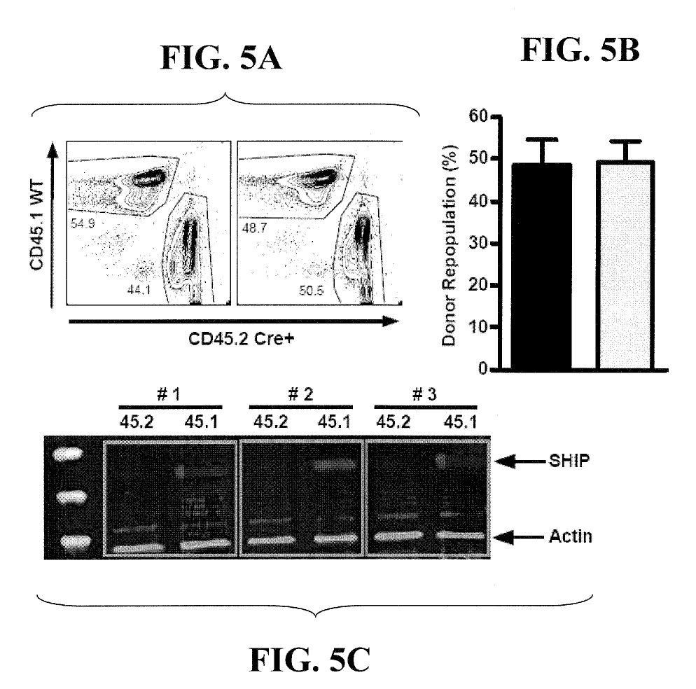

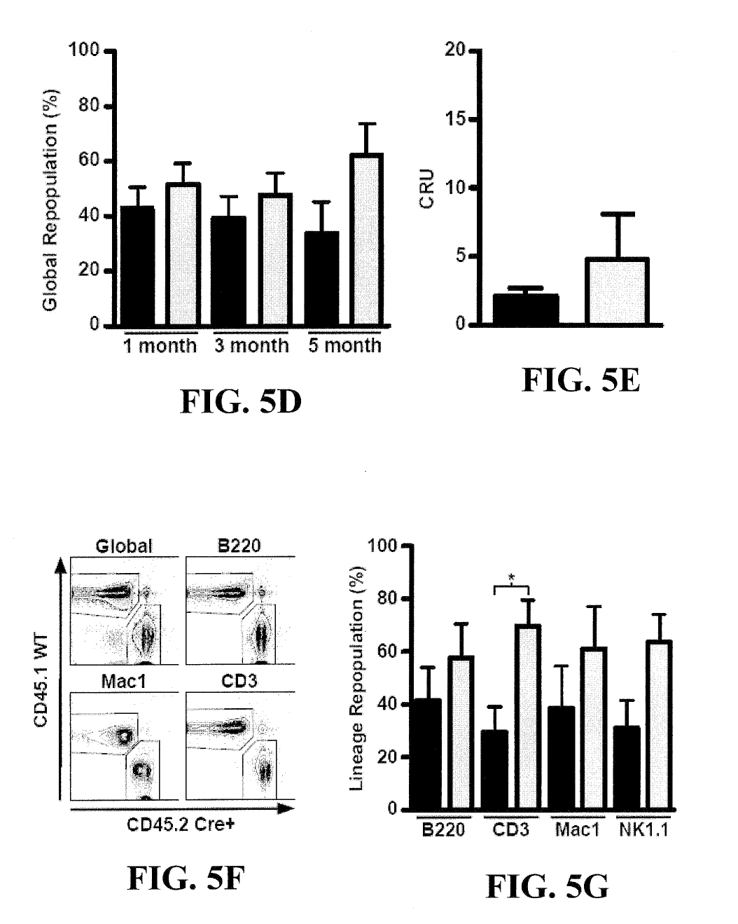

To determine if the repopulation defect we observed previously for SHIP^{-/-} HSC is due to an inability to home efficiently to the BM HSC niche, we developed an in situ SHIP deletion model where SHIP expression is ablated after achieving HSC engraftment, bypassing the requirement of SHIP for BM homing. In this model we co-transplant an equal dose of SHIP-competent BM cells in which induction of Cre-mediated deletion of SHIP is not possible and thus this model enables quantitation of competitive repopulating activity (CRU) for HSC rendered SHIP-deficient in situ. A similar approach was previously used to study the role of Rho family proteins in HSC homing and repopulation (Cancelas et al., 2005). In our in situ SHIP deletion model CD45.1⁺45.2⁺ hemizygous recipients are lethally irradiated and transplanted with equal numbers of CD45.2⁴CD45.1⁻ MxCreSHIP^{flox/flox} and CD45.1⁺CD45.2⁺ SHIP^{-/-} WBM cells. In these chimeras, equivalent repopulation from both the MxCreSHIP^{flox/flox}(CD45.2⁺CD45.1⁻) and SHIP^{+/+}(CD45.1 CD45.2) BM donors was observed at 60 days post-transplant, indicating comparable levels of HSC engraftment from each BM donor (FIGS. 5A and 5B). We then administered three intra-peritoneal polyI:C injections to induce deletion of SHIP in the MxCreSHIP^{flox/flox} portion of these BM chimeras. Two months after polyI:C treatment, CD45.2⁺CD45.1⁻ and CD45.1⁺CD45.2⁻ cells from the PB were isolated by cell sorting and the level of SHIP expression in each sorted population was assessed by Western blot analysis. SHIP deletion is highly efficient in polyI:C-treated MxCreSHIP^{flox/flox} cells as was indicated by the near complete absence of SHIP expression in the CD45.2⁺CD45.1⁻ cells derived from MxCreSHIP^{flox/flox} HSC (data not shown). To further confirm in situ deletion of SHIP in this model, we harvested spleens from all the chimeras upon their termination at 5 months post-transplant. Western blot analysis of CD45.2⁺CD45.1⁻ cells isolated by flow cytometry (FIG. 5C) confirmed nearly complete ablation of SHIP expression in the hematopoietic compartment derived from MxCreSHIP^{flox/flox} HSC present in these BM chimeras.

At monthly intervals we monitored the degree of multi-lineage repopulation by CD45.2⁺CD45.1⁻ (SHIP-deficient) vs. CD45.1⁺CD45.2⁻ (SHIP-competent) cells in these chimeras. We found that SHIP-deleted HSC retain the ability to efficiently perform long-term, multi-lineage repopulation at levels comparable to the competing SHIP-competent HSC present in these chimeras. We observed no significant difference in global hematopoietic repopulation (FIG. 5D) or CRU activity (FIG. 5E) between in situ SHIP-deleted and WT HSC up to 5 months following ablation of SHIP expression. In addition, repopulation of both the lymphoid and myeloid arms of hematopoiesis was observed out to 5 months following induction of SHIP-deficiency (FIG. 5F). Thus, when HSC are established in situ and then rendered SHIP-deficient this does not significantly compromise their potential for long-term, multi-lineage repopulation. However, we did observe a small, but significant, reduction in T lymphocyte reconstitution in the CD45.2⁺CD45.1⁻ SHIP-deficient compartment despite normal repopulation of the B and NK lymphoid lineages (FIG. 5G). Consistent with this finding, a reduction in peripheral T cell numbers is also observed in germline SHIP^{-/-} mice (Helgason et al., 1998). Because decreased T cell production is observed only in the SHIP-deficient portion of these chimeras and not in the other lymphoid lineages, we conclude that SHIP is required for the efficient development of T lymphocytes and this requirement is intrinsic to the T cell lineage.

### Example 2

### SHIP-Deficient HSC Derived from a SHIP-Competent Niche have Normal Self-Renewal Capacity

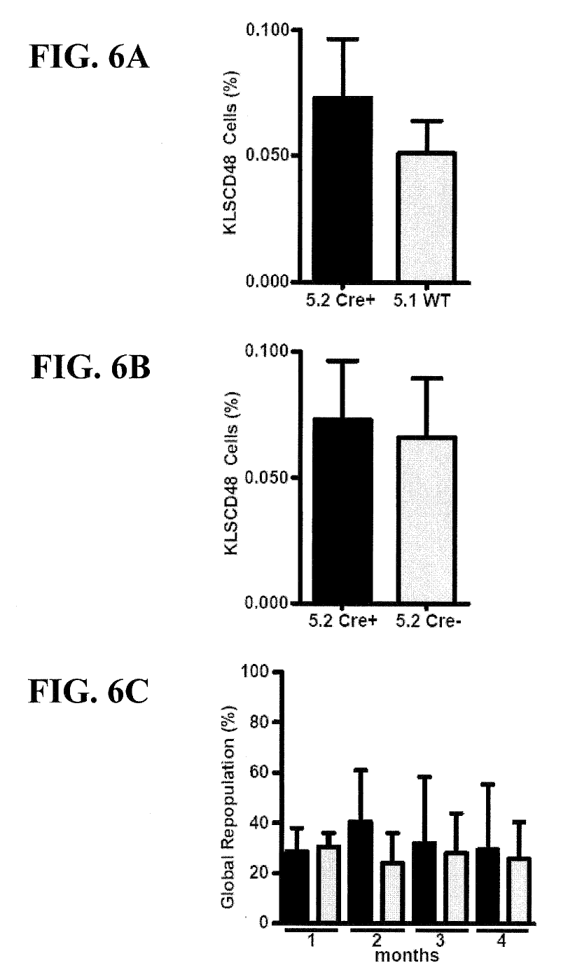

Analysis of the above chimeras demonstrated long-term, multi-lineage repopulation from the SHIP-deficient HSC compartment is intact when HSC are resident in a SHIP-competent niche. This finding suggested that self-renewal capacity might also be intact in the SHIP-deficient HSC. To compare the self-renewal capacity of the SHIP-deficient and SHIP-competent HSC in these chimeras we performed serial BM transfers from the initial chimeras to secondary recipients. Whole BM was harvested from the chimeras described above at 5 months following SHIP deletion and was then transplanted into lethally irradiated CD45.1⁺CD45.2⁺ secondary hosts. Prior to these serial transfers we first compared the proportion of SHIP-deficient (CD45.2⁺CD45.1⁻) to SHIP-competent (CD45.1⁺CD45.2⁻) cells in the HSC compartment by multi-parameter flow cytometric analysis of CD45.1 vs. CD45.2 staining on Kit⁺Lin⁻Sca1⁺CD48⁻ (KLSCD48) cells. This analysis revealed a statistically comparable level of contribution to KLSCD48 HSC by the SHIP-deficient and SHIP-competent HSC within the MxCreSHIP^{flox/flox}:WT-Ly5.1 BM chimeras (FIG. 6A). SHIP-deficient HSC in these MxCreSHIP^{flox/flox}:WT-Ly5.1 BM chimeras were also found to represent a statistically comparable proportion of the HSC compartment as compared to SHIP^{flox/flox} HSC in the SHIP^{flox/flox}:WT-Ly5.1 BM control chimeras analyzed in parallel (FIG. 6B). We then monitored global repopulation in the secondary hosts for a period of four months and found that SHIP-deficient HSC from MxCreSHIP^{flox/flox}:WT-Ly5.1 BM chimeras competed effectively in donor blood cell repopulation as compared to the WT-Ly5.1 HSC present in the same BM inoculum (FIG. 6C), demonstrating that SHIP-deficient HSC can effectively home and engraft upon transplant when they are derived from a SHIP-competent HSC niche. Thus, SHIP expression is not an intrinsic requirement for HSC homing to BM and self-renewal.

### Example 3

### Systemic Induction of Ship-Deficiency in Adult Physiology is Detrimental to the Repopulating Capacity of BM HSC

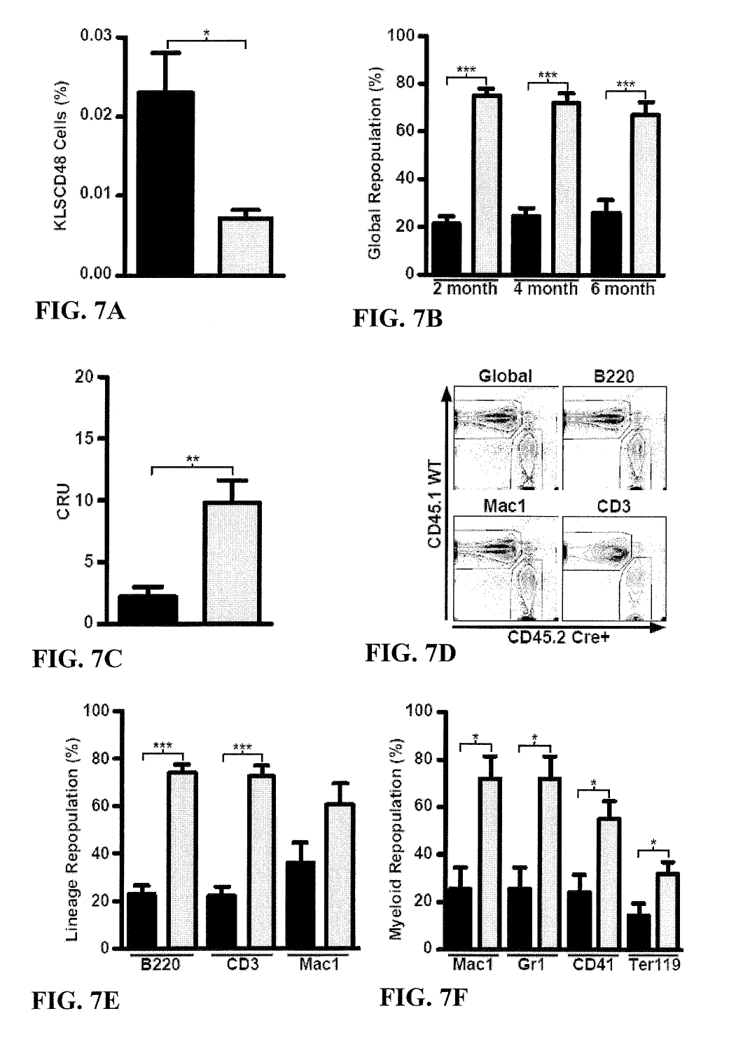

SHIP is expressed in embryonic, fetal and adult tissues, including mesenchymal, endothelial and hematopoietic lineages (Tu et al., 2001; Desponts et al., 2006b; Damen et al., 1996; Geier et al., 1997). Cells from all three of these tissues participate directly or indirectly in the HSC niche in BM and thus their development may be impaired or altered during ontogeny in mice with germline SHIP-deficiency. Therefore, the HSC disruptions we observe could be due to developmental alterations of the niche by SHIP-deficiency. Alternatively, SHIP could be required in adult physiology for the normal function of the niche. To test the latter possibility, we induced systemic SHIP-deficiency in adult mice and then asked whether this also impairs HSC function. Induction of SHIP-deficiency in the previous chimeras showed no intrinsic requirement of SHIP for BM horning, repopulation or self-renewal by HSC and thus, in the setting of induced, but systemic SHIP-deficiency we are testing whether SHIP-deficiency impairs the HSC niche in BM. For these studies, BM was harvested from MxCreSHIP^{flox/flox} mice that had undergone systemic SHIP-ablation by three polyI:C injections. Induction of SHIP-deficiency was confirmed by Western blot analysis of PBMC (data not shown). Prior to transplant we observed that the KLSCD48 HSC compartment in mice with induced SHIP-deficiency is significantly expanded relative to SHIP^{flox/flox} controls that underwent an equivalent regimen of polyI:C injections (FIG. 7A). Furthermore, the absolute number of KLSCD48 HSC in mice with induced SHIP-deficiency is significantly expanded compared to SHIP^{flox/flox} controls (data not shown). This expansion seems to be restricted to more primitive KLSCD48 and KLS progenitor cells and was not found in cKit⁺Lineage⁻ cells (data not shown). Because we observed no significant expansion of the SHIP-deficient HSC compartment in chimeras with a SHIP-competent niche (FIGS. 5A-5G), we conclude then that SHIP expression is required by the adult BM niche to limit HSC compartment size. To analyze the repopulating ability of BM HSC in mice with systemic induction of SHIP deficiency we co-transplanted equal numbers of WBM cells from SHIP-deleted (CD45.2⁺CD45.1⁻) and SHIP-competent(CD45.1⁺CD45.2⁻) donors into lethally irradiated WT(CD45.2⁺CD45.1⁺) recipients. As before, monthly peripheral blood monitoring was used to assess engraftment. Using both global repopulation (FIG. 7B) and CRU (FIG. 7C) as measures of engraftment, the HSC from donors with induced SHIP-deficiency did not compete effectively against HSC from SHIP-competent donors. Furthermore, multi-lineage reconstitution appears to be significantly compromised for all lymphoid lineages in the PB (FIGS. 7D, 7E) and in myelo-erythroid lineages in the BM (FIG. 7F). However, despite significantly decreased myelopoiesis, megakaryopoiesis and erythropoiesis in the BM by HSC from mice with induced SHIP-deficiency (FIG. 7F), we observed statistically comparable myeloid reconstitution in the peripheral blood and spleen from these HSC (FIG. 7E and data not shown). This sustained peripheral myelopoiesis by the SHIP-deficient BM graft that occurs in spite of significantly compromised central myelopoiesis is likely due to robust extramedullary myelopoiesis that we and others observe in the spleens of SHIP^{-/-} mice (Helgason et al., 1998) (Hazen & Kerr, unpublished data).

### Example 4

### A SHIP-Deficient BM Microenvironment Expands HSC and Reduces Surface Expression of CXCR4

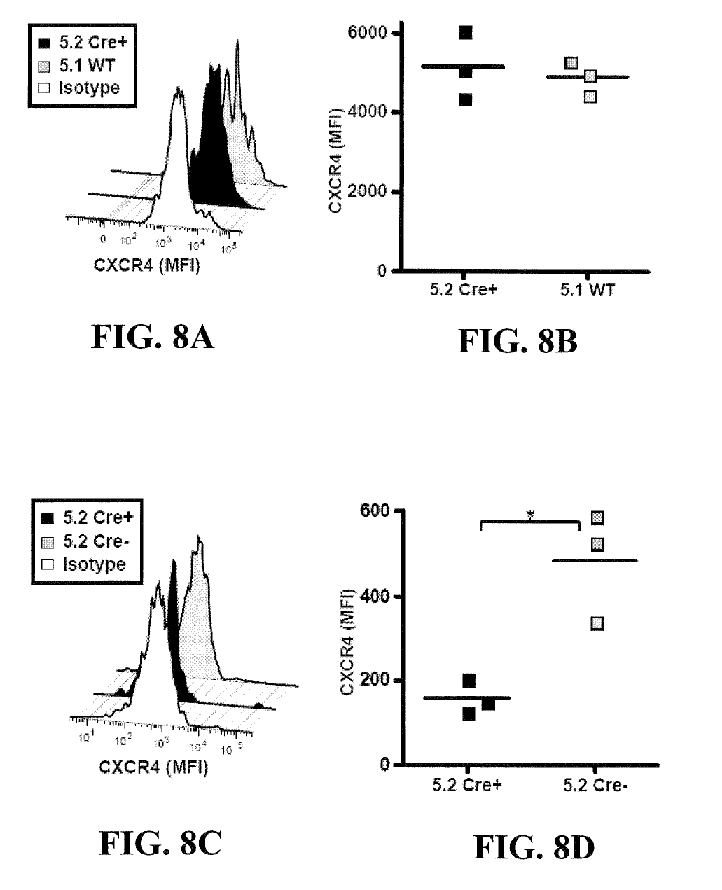

Expansion and functional impairment of HSC in mice following the induction of systemic SHIP-deficiency described above was reminiscent of our previous findings with HSC from germline SHIP^{-/-} mice (Desponts et al., 2006a). To examine whether CXCR4 surface expression on HSC might also be altered by the SHIP status of the BM niche, we assessed CXCR4 surface expression for in situ SHIP-deleted HSC where the niche remains SHIP-competent and in HSC from mice following induction of systemic SHIP-deficiency where the BM microenvironment is also rendered SHIP-deficient. In the in situ SHIP-deleted model described in FIGS. 5A-5G, we found that the SHIP-deficient HSC do not show significant reductions in CXCR4 surface expression as compared to SHIP-competent HSC present in the same mice (FIGS. 8A, 8B). However, when we analyzed the surface expression of CXCR4 on HSC in mice with induced systemic SHIP-deficiency, we found that the CXCR4 surface expression is significantly reduced relative to SHIP^{flox/flox} controls that received an equivalent regimen of polyI:C (FIGS. 8C, 8D). As shown in FIGS. 7A-7F, KLSCD48 HSC are also significantly expanded in mice when systemic SHIP-deficiency is induced. Expansion of the HSC compartment and down-modulation of CXCR4 are triggered by SHIP-deficiency, demonstrating regulation of these HSC properties by the BM niche during adult physiology.

### Example 5

### Production of Soluble Factors that Influence HSC Behavior are Altered in SHIP-Deficient Mice

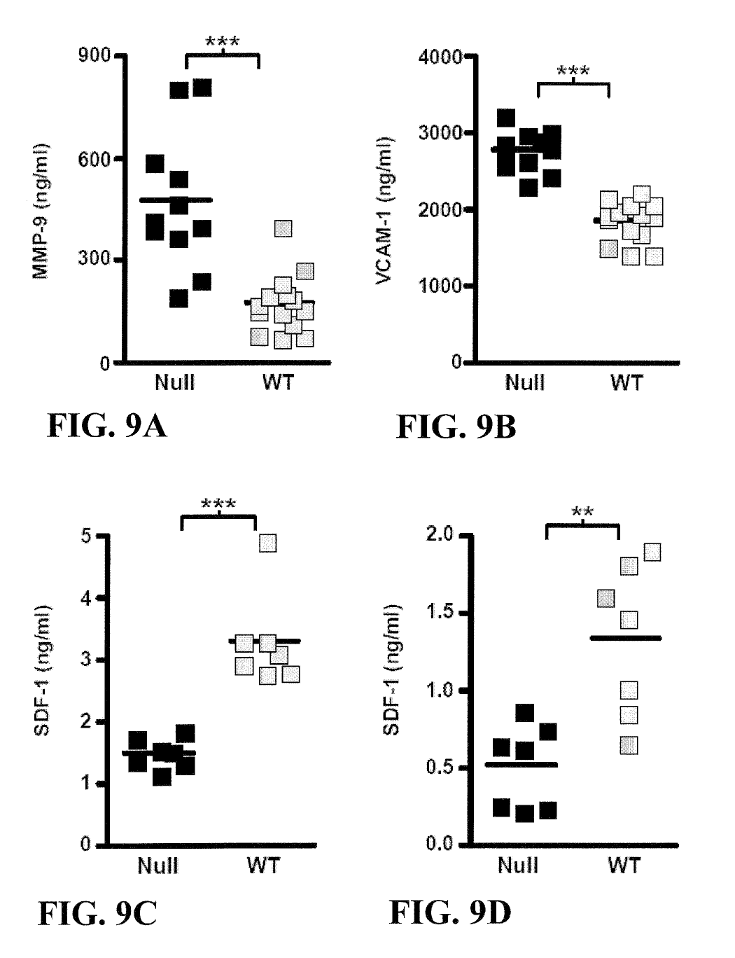

Although the BM niche can influence the behavior of HSC by elaborating cell hound ligands for HSC receptors, it also mediates effects through the production of soluble factors that influence HSC proliferation, survival, mobilization, and BM homing. Consistent with our proposed disruption of niche function by SHIP-deficiency, SHIP^{-/-} mice exhibit significantly increased plasma levels of several growth and survival factors for HSC (TPO, G-CSF, IL-6) (Table 2). The increase in these factors and others may contribute to the increased number of HSC present in both the medullary and extramedullary compartments of SHIP^{-/-} mice. We also observe increased concentrations of soluble factors that promote HSC mobilization, including G-CSF, IL-5 (Table 2) MMP-9 (FIG. 9A) and soluble VCAM-1 (FIG. 9B). In addition, homing and retention of HSC by the SHIP-deficient BM niche may be compromised by reduced production of SDF-1/CXCL12. We find significantly reduced SDF-1/CXCL12 in both the blood and BM plasma of SHIP-deficient mice (FIGS. 9C, 9D). SDF-1/CXCL12 is produced by both the osteoblastic and vascular niche in BM (Ara et al., 2003; Petit et al., 2002). In fact, decreased SDF-1 production in these SHIP-deficient BM niches could be triggered by increased G-CSF production, as G-CSF is known to reduce SDF-1 expression in BM niche cells (Semerad et al., 2005). Taken together, these findings provide evidence for several significant perturbations in the SHIP-deficient BM microenvironment.

TABLE 2
Disrupted cytokine distribution in peripheral blood of SHIP-deficient mice

| Cytokine | SHIP −/− (SEM, N) | WT (SEM, N) | P value |
|---|---|---|---|
| TPO (ng/ml) | 13.36 (±0.3636, N = 11) | 11.79 | 0.0007 |
| | | (±0.2143, N = 14) | |
| G-CSF | 749.1 (±39.13, N = 4) | 161.9 | <0.0001 |
| (pg/ml) | | (±24.62, N = 4) | |
| IL-6 (pg/ml) | 39.82 (±6.201, N = 11) | 13.67 | 0.0001 |
| | | (±0.5205, N = 13) | |
| IL-5 (ng/ml) | 0.2147 (±0.02350, N = 11) | 0.1511 | 0.0314 |
| | | (±0.01634, N = 14) | |

Various factors were measured in serum obtained from PB by ELISA. The data is pooled from multiple assays derived from at least 4 different plasma samples for each genotype. Significance was established using the unpaired Student's T test.

### Example 6

### A Role for Ship in BM Microenvironment Signaling and Function

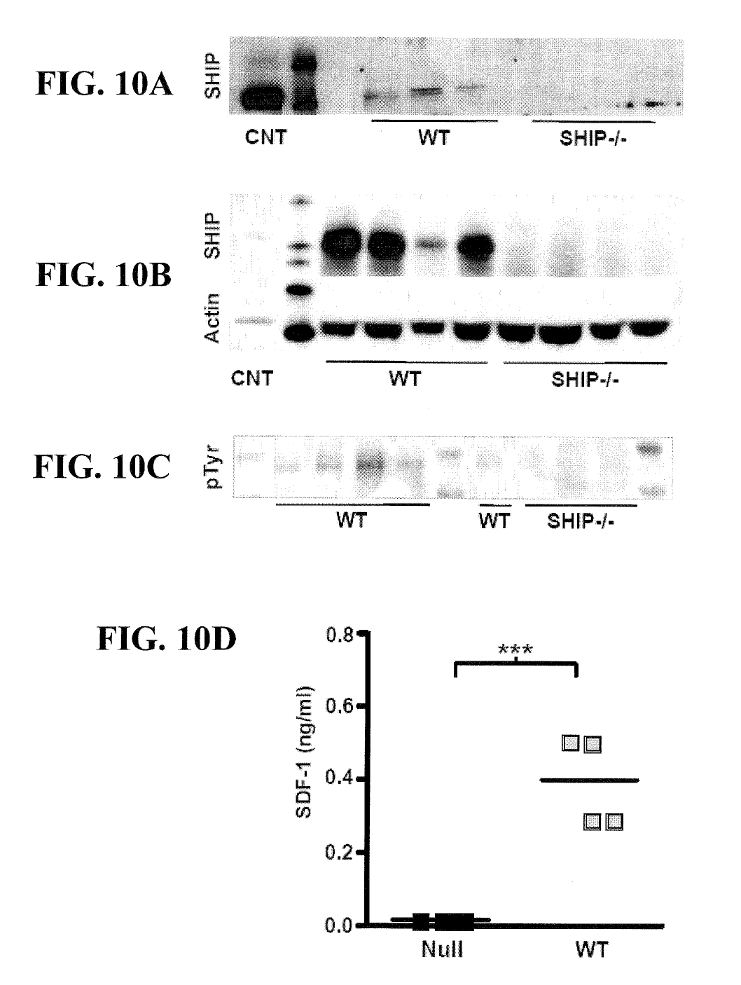

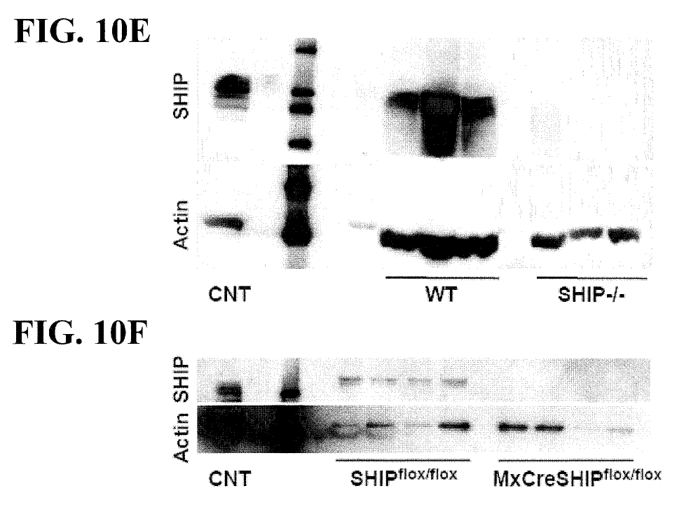

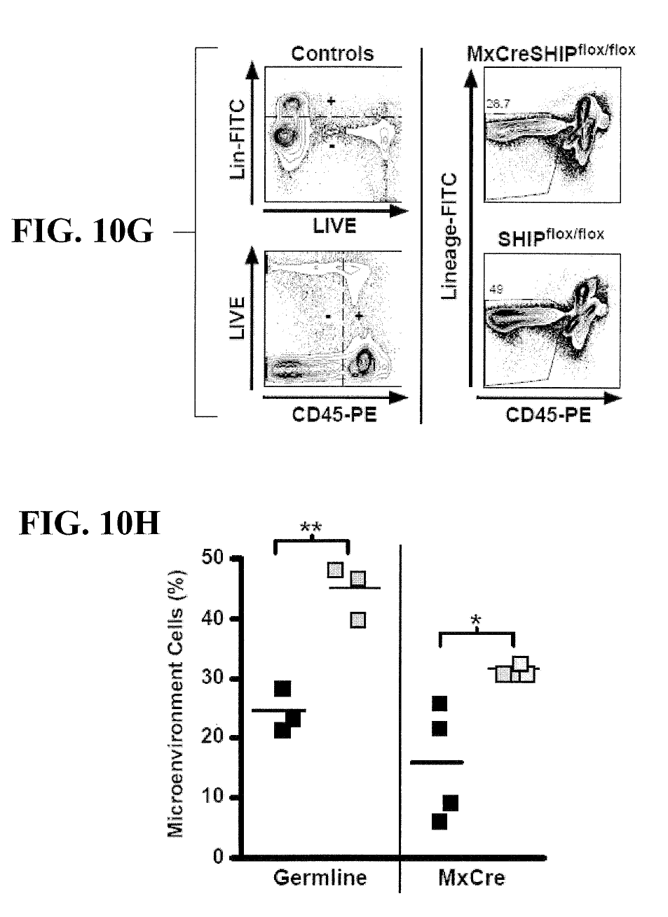

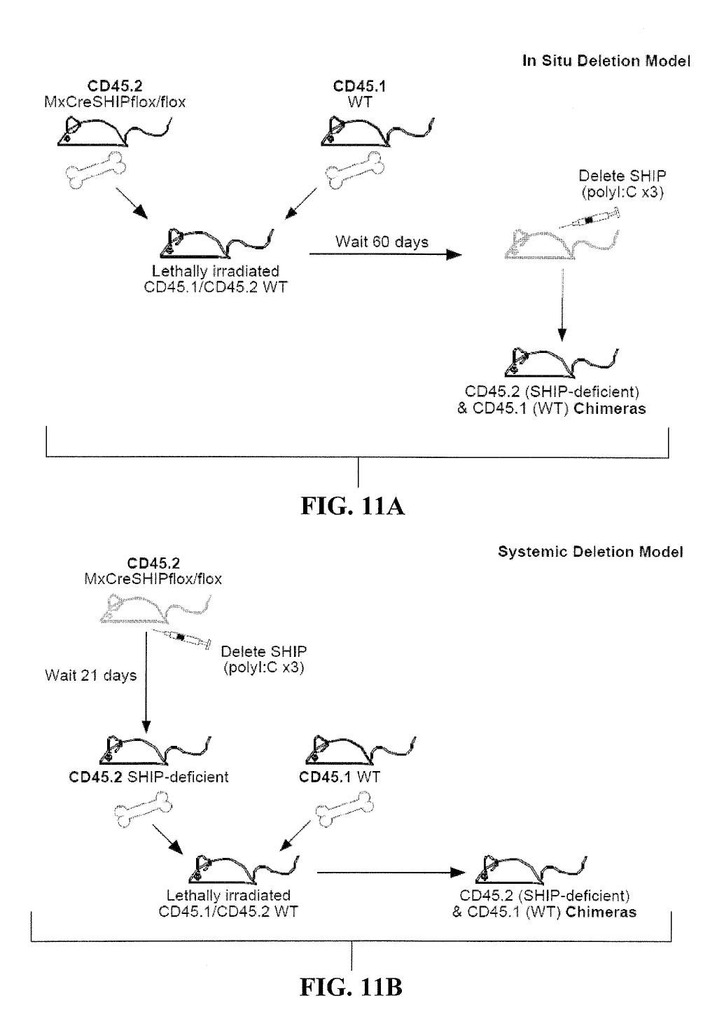

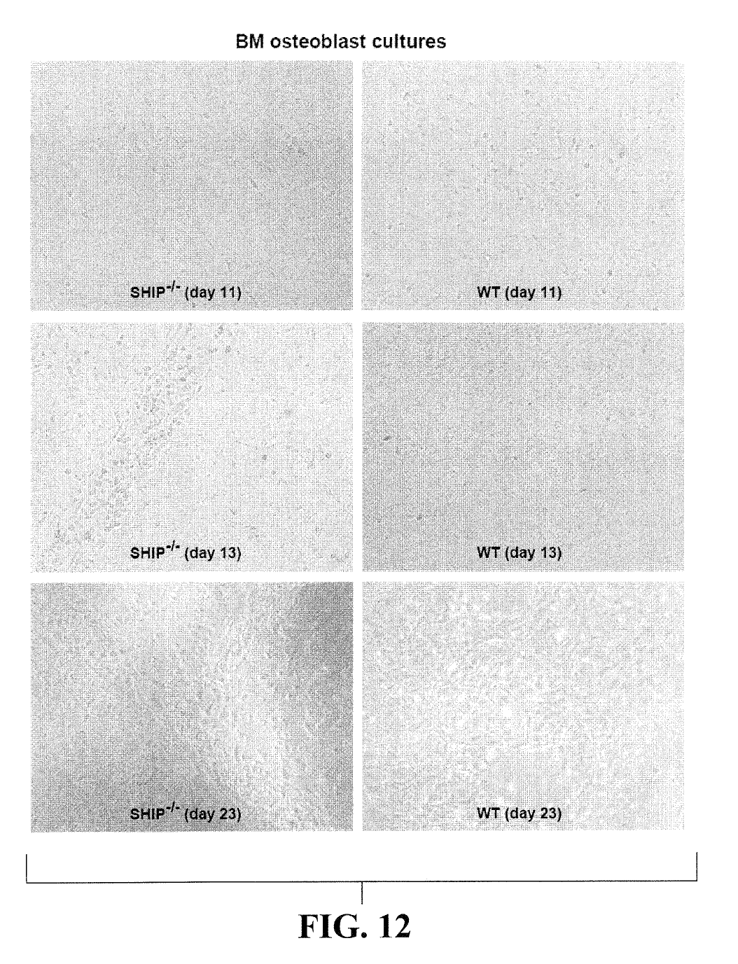

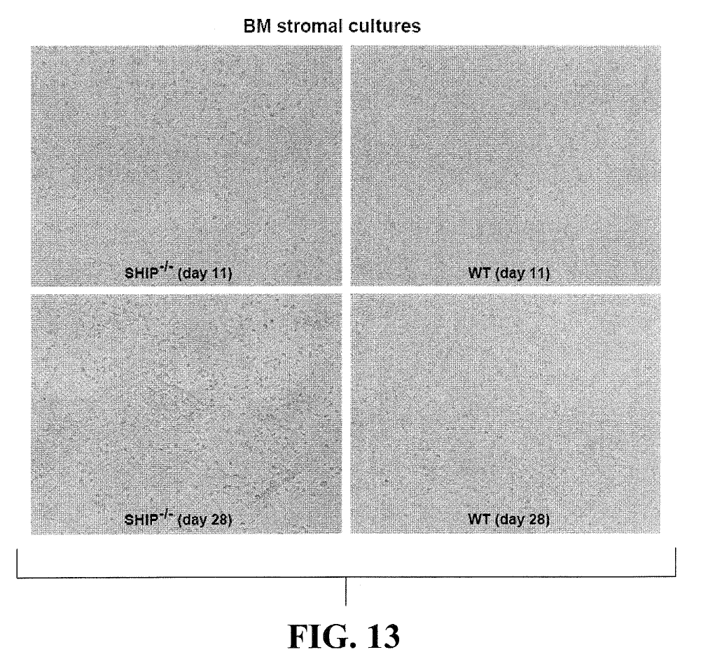

Our transplantation studies, described above, indicated SHIP deficiency disrupts the BM niche that supports normal HSC function and BM homing. SHIP is expressed primarily in hematopoietic tissues in both humans and mice; however, direct analysis of SHIP expression in cells that constitute BM niche cell types was not directly tested (Damen et al., 1996; Geier et al., 1997; Lioubin et al., 1996; Kerr et al., 1996; Ware et al., 1996). We then sought more direct evidence of a role for SHIP in the BM microenvironment by assessing whether it is expressed in cells that constitute key non-hematopoietic cell types in the BM niche. Thus, we prepared osteoblast (OB) and stromal cell cultures from SHIP^{-/-} and WT BM (FIGS. 12 and 13). There were no substantive differences in morphology of stromal cells between the two genotypes (FIG. 13). However, in OB cultures we consistently observe readily apparent differences with SHIP^{-/-} OB exhibiting elongated and non-randomly oriented protrusions (FIG. 12: day 11) and organized growth along axes (FIG. 12: days 13 and 23). We readily detect SHIP protein expression in WT stromal (FIG. 10A) and OB cultures (FIG. 10B). In addition, we detect tyrosine phosphorylation of SHIP in WT BM stromal cells indicating it participates in signaling pathways active in cells of the BM microenvironment (FIG. 10C). To assess a key BM microenvironment function, we then analyzed the supernatants of multiple independent SHIP^{-/-} and WT stromal cultures for SDF-1 production and found profoundly compromised SDF-1 production in all SHIP^{-/-} cultures analyzed as compared to WT cultures analyzed in parallel (FIG. 10D). Thus, SDF-1 production by BM niche cells is significantly impaired by SHIP-deficiency. As SDF-1 is pivotal for BM homing and retention of HSC, the impaired production of SDF-1 by SHIP^{-/-} stroma is consistent with increased peripheralization of HSC to the blood and spleen of SHIP^{-/-} mice (Desponts et al., 2006a). These results demonstrate a direct requirement by BM microenvironment cells for SHIP in a niche function required for BM recruitment and retention of HSC.

A lack of CD45 and hematopoietic lineage markers is used to delineate mesenchymal stem/progenitor cells and endothelial cells in the BM microenvironment (Sudres et al., 2006). Consistent with our detection of SHIP expression in cultures of BM niche cells shown above, we confirmed that SHIP protein expression is also readily detected in freshly isolated CD45⁻Lin⁻ cells in the BM compartment of both WT and SHIP^{flox/flox} (FIGS. 10E, 10F). In addition, we find there are significantly less CD45⁻Lin⁻ cells in the BM of mice that have germline or induced SHIP deficiency, relative to their SHIP competent counterparts (FIGS. 10G, 10H). Thus, SHIP expression is also required for the normal production of cells in the non-hematopoietic BM compartment during adult physiology.

### Example 7

### SHIP Knockdown Using Interfering RNA

Four potential interfering RNA sequences targeting human SHIP were obtained from Open Biosystems:

| | |
|---|---|
| siRNA sequence H1: | |
| AAGGAAUUGCGUUUACACUUA | (SEQ ID NO: 16) |
| siRNA sequence H2: | |
| AAAAUUGCGUUUACACUUACA | (SEQ ID NO: 17) |

shRNA Sequence 63332:

(SEQ ID NO: 18)

TGCTGTTGACAGTGAGCGAGGCTTCCAGAAGAGCATCTTATAGTGAAG
CCACAGATGTATAAGATGCTCTTCTGGAAGCCCTGCCTACTGCCTCGGA

shRNA Sequence 63331:

(SEQ ID NO: 19)

TGCTGTTGACAGTGAGCGAGCCCATATCACCCAAGAAGTTTAGTGAAG
CCACAGATGTAAACTTCTTGGGTGATATGGGCGTGCCTACTGCCTCGGA

The underlined portion of shRNA 63332 (SEQ ID NO:18): GCTTCCAGAAGAGCATCTTAT (SEQ ID NO:20), and shRNA 63331 (SEQ ID NO:19): GCCCATATCACCCAAGAAGTTT (SEQ ID NO:21), represent the target sequences in human SHIP. The T nucleotides in shRNA 63332 and 63331 can also be replaced by U nucleotide. The other non-underlined portions are the loop and termini of the shRNA.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 4,522,811
U.S. Pat. No. 4,987,071
U.S. Pat. No. 5,011,912
U.S. Pat. No. 5,087,617
U.S. Pat. No. 5,093,246
U.S. Pat. No. 5,116,742
U.S. Pat. No. 5,135,917
U.S. Pat. No. 5,144,019
U.S. Pat. No. 5,168,053
U.S. Pat. No. 5,176,996
U.S. Pat. No. 5,180,818
U.S. Pat. No. 5,190,931
U.S. Pat. No. 5,272,262
U.S. Pat. No. 5,328,470
U.S. Pat. No. 5,843,509
U.S. Pat. No. 6,090,621
U.S. Pat. No. 6,096,720
U.S. Pat. No. 6,168,587
U.S. Pat. No. 6,194,389
U.S. Pat. No. 6,468,798
U.S. Pat. No. 6,471,996
U.S. Pat. No. 6,472,375
U.S. Pat. No. 6,506,559
U.S. Pat. No. 6,649,192
U.S. Published Application No. 2002/193579
U.S. Published Application No. 2006/0223749
WO 01/68836
WO 01/75164
WO 01/92513
WO 97/10252
WO 97/12039
WO 99/32619
Abbas-Terki, T. et al. Hum Gene Ther., 2002, 13:2197-201
Alter B. P., Inherited bone marrow failure syndromes, In: Nathan D. B. et al., Nathan and Oski's Hematology of Infancy and Childhood, 6^{th} edition, Philadelphia, Pa., WB Saunders, 2003:280-365
Alter B. P., Inherited forms of aplastic anemia: the inherited bone marrow failure syndromes (IBMFS), In: Burg F. D. et al., Gellis and Kagan's Current Pediatric Therapy, Philadelphia, Elsevier, 2005
Altschul et al., Nucleic Acids Res. 25:3389-3402 (1997)
Altschul, et al. J. Mol. Biol. 215:403-10 (1990)
Amarzguioui M. and H. Prydz, Biochem. Biophys. Res. Commun., 2004, 316:1050-1058
Ara T, Tokoyoda K, Sugiyama T, Egawa T, Kawabata K, Nagasawa T. Long-term hematopoietic stern cells require stromal cell-derived factor-1 for colonizing bone marrow during ontogeny. Immunity. 2003; 19:257-267.
Arnett T, Henderson B eds. Methods in Bone Biology. London: Chapman and Hall; 1998.
Asnagli et al., J. Immunol. 168:4268-4271, 2002
Avecilla, S. T. et al., Nat Med., 2004, 10:64-71
Bagella et al., J. Cell. Physiol. 177:206-213 (1998)
Bagheri S. et al., Curr. Mol. Med., 2004, August, 4(5):489-506
Bakker A, Klein-Nulend J. Osteoblast isolation from murine calvariae and long bones. Methods Mol Med. 2003; 80:19-28.
Bartel, D. and Szostak, J. W. Science 261:1411-1418 (1993)
Barton, G. M. and Medzhitov, R. Proc Natl Acad Sci USA, 2002, 99:14943-5
Bass, Nature 411:428-29 (2001)
Bernstein et al., (2001a) RNA 7:1509
Bernstein et al., (2001b) Nature 409:363-66
Bonetta L. “RNAi: Silencing never sounded better”, Nature Methods, October, 2004, 1(1):79-86
Borkhardt, A. Cancer Cell, 2002, 2:167-8
Brauweiler A, Tamir I, Dal Porto J, et al. Differential regulation of B cell development, activation, and death by the src homology 2 domain-containing 5′ inositol phosphatase (SHIP). J Exp Med. 2000; 191:1545-1554.
Bristeau et al., Gene 274:283-291, 2001
Brummelkamp et al., Science 296:550-553 (2002)
Calegari et al., Proc. Natl. Acad. Sci. USA 99(22):14236-40 (2002)
Calvi L M, Adams G B, Weibrecht K W, et al. Osteoblastic cells regulate the haematopoietic stem cell niche. Nature. 2003; 425:841-846.
Cancelas J A, Lee A W, Prabhakar R, Stringer K F, Zheng Y, Williams D A. Rac GTPases differentially integrate signals regulating hematopoietic stem cell localization. Nat Med. 2005; 11:886-891.
Carthew et al., Curr. Opin. Cell Biol. 13:244-48 (2001)
CDC Fact Sheet (2003), Acute Radiation Syndrome, Center for Disease Control and Prevention, U.S. Department of Health and Human Services; Edwards, R. Risk of radioactive “dirty bomb” growing, New Scientist online, June 2004
Chae, S-S. et al., J. Clin. Invest., 2004, 114:1082-1089
Chapter XII-Biological Effects, In: The Effects of Nuclear Weapons, Third Edition, compiled and edited by Glasstone S, and P. J. Dolan, United States Department of Defense and the Energy Research and Development Administration, 1977
Chen et al., Proc. Natl. Acad. Sci. USA 91:3054-3057 (1994)
Chen J., Clinical Medicine & Research, 2005, 3(2):102-108
Chen, A. A. et al. Nucleic Acids Res., 2005, 33:e190
Chemock, R. D. et al. Blood, 2001, 97:608-615
Chiu et al., Mol. Cell. 10:549-561 (2002)
Chiu et al., Oncogene 19:6229-6239, 2000
Clawson G. A. et al., Gene Ther., 2004, September, 11(17):1331-1341
Clemens et al., Proc. Natl. Acad. Sci. USA 97:6499-6503 (2000)
Clement S, Krause U, Desmedt F, et al. The lipid phosphatase SHIP2 controls insulin sensitivity. Nature. 2001; 409:92-97.
Clusel et al., 1993 Nucl. Acids Res. 21:3405-11
Colella M., et al., An introduction to radiological terrorism, Australian Journal of Emergency Management, May, 2005, 20(2):9-17
Cortez et al., J. Neurosci. Res. 59:39-46, 2000
Damen J E, Liu L, Rosten P, et al. The 145-kDa protein induced to associate with She by multiple cytokines is an inositol tetraphosphate and phosphatidylinositol 3,4,5-triphosphate 5-phosphatase. Proc Natl Acad Sci USA. 1996; 93:1689-1693.
Davis et al., 1986 Basic Methods in Molecular Biology
Desponts C, Hazen A L, Paraiso K H, Kerr W G. (2006a) SHIP deficiency enhances HSC proliferation and survival but compromises homing and repopulation. Blood. 107:4338-4345.
Desponts C, Ninos J M, Kerr W G. (2006b) s-SHIP associates with receptor complexes essential for pluripotent stem cell growth and survival. Stem Cells Dev.; 15:641-646.
Devroe, E. and Silver, P. A. BMC Biotechnol., 2002, 2:15
Dorn, G. et al. Nucleic Acids Res., 2004, 32, e49
Downward, J. BMJ, 2004, 328:1245-1248
Drachman, J. G. et al. (1997a) Blood, 89:483-492
Drachman, J. G. et al. (1997b) Proc Natl Acad Sci USA, 94:2350-2355
Dritschilo, A. and Sherman, D. Environ. Health Perspee, 1981, 39:59-64
Dykxhoorn, D. M. and Lieberman J., Annu. Rev. Med., 2005, 56:401-423
Dykxhoorn, D. M. et al. Gene Ther., 2006, epub ahead of print
Elbashir et al., (2001a) EMBO 1, 20:6877-6888
Elbashir et al., (2001b) Genes Dev. 15:188-200
Elbashir et al., (2001c) Nature 411:494-498
Elbashir S M. et al., (2002) Methods, 26:199-213
El-Meanawy et al., Am. J. Physiol. Renal Physiol. 279:F383-392, 2000
Fattal et al., J. Control Release 53(1-3):137-43 (1998)
Fire et al., Nature 391:806-11 (1998)
Fire, A. Trends Genet., 1999, 15:358-363
Fougerolles, A. et al., Methods Enzymol., 2005, 392:278-296
Franz et al., Circ. Res. 73:629-638, 1993
Freedman M. H., Oncologist, 1996, 1:354-360
Fuchs, U. et al. Curr. Mol. Med., 2004, 4:507-517
Gaultier et al., Nucleic Acids. Res. 15:6625-6641 (1987)
Geddis, A. E. et al. J Biol Chem., 2001, 276:34473-34479
Geier S J, Algate P A, Carlberg K, et al. The human SHIP gene is differentially expressed in cell lineages of the bone marrow and blood. Blood. 1997; 89:1876-1885.
Ghansah T, Paraiso K H, Highfill S, et al. Expansion of myeloid suppressor cells in SHIP-deficient mice represses allogeneic T cell responses. J. Immunol. 2004; 173:7324-7330.
Gilmore, I. R. et al., J. Drug Target., 2004, 12(6):315-340
Giuriato, S. et al. Biochem J., 2003, 376:199-207
Giuriato, S. et al. J Biol Chem., 1997, 272:26857-26863
Gluzman, Cell 23:175 (1981)
Godard et al., Eur. J. Biochem. 232(2):404-10 (1995)
Gong D. and J. E. Ferrell Jr., TRENDS in Biotechnology, 2004, 22(9):451
Goodchild J., Curr. Opin. Mol. Ther., 2004, April, 6(2):120-128
Grabarek et al., BioTechniques 34:73544 (2003)
Grassi G. et al., Curr. Pharm. Biotechnol., 2004, August, 5(4):369-386
Gupta N. et al., J. Exp. Med., 1997, 186(3):473-478
Gupta, S. et al. PNAS, 2004, 101:1927-1932
Hamada, T. et al. J Exp Med., 1998, 188:539-548
Hamajima et al., Clin. Immunol. Immunopathol. 88(2):205-10 (1998)
Hammond, S. M. et al. Science, 2001, 293:1146-1150
Hannon, G. J. Nature, 2002, 418:244-51
Harborth et al., J. Cell Sci. 114:4557-65 (2001)
Harrison D E, Jordan C T, Zhong R K, Astle C M. Primitive hemopoietic stem cells: direct assay of most productive populations by competitive repopulation with simple binomial, correlation and covariance calculations. Experimental Hematology. 1993; 21:206-219.
Haselhoff and Gerlach Nature 334:585-591 (1988)
Hattori, K. et al. Blood, 2001, 97:3354-3360
Helene, C. Ann. N.Y. Acad. Sci. 660:27-36 (1992)
Helene, C. Anticancer Drug Des. 6:569-84 (1991)
Helgason C D, Antonchuk J, Bodner C, Humphries R K. Homeostasis and regeneration of the hematopoietic stem cell pool are altered in SHIP-deficient mice. Blood. 2003; 102:3541-3547.
Helgason C D, Damen J E, Rosten P, et al. Targeted disruption of SHIP leads to hemopoietic perturbations, lung pathology, and a shortened life span. Genes Dev. 1998; 12:1610-1620.
Helgason C D, Kalberer C P, Damen J E, et al. A dual role for Src homology 2 domain-containing inositol-5-phosphatase (SHIP) in immunity: aberrant development and enhanced function of b lymphocytes in ship −/− mice. J Exp Med. 2000; 191:781-794.
Hendry P. et al., BMC Chem. Biol., 2004, December, 4(1):1
Hopp et al., Bio/Technology 6:1204, 1988
Huber et al., (1998a) Proc. Natl. Aca. Sci. USA, 95(19):11330-11335
Huber M, Helgason C D, Scheid M P, Duronio V, Humphries R K, Krystal G. (1998b) Targeted disruption of SHIP leads to Steel factor-induced degranulation of mast cells. Embo J.; 17:7311-7319.
Huppi, K. et al. Mol. Cell, 2005, 17:1-10
Hutvagner and Zamore, Curr. Opin. Genet. Dev.: 12, 225-232 (2002)
Ichim, T. E. et al. Am. J. Transplant, 2004, 4:1227-1236
Inoue et al. (1987a) FEBS Lett., 215:327-330
Inoue et al. (1987b) Nucleic Acids Res. 15:6131-6148
Jacque (2002)
Jana, S. et al. Appl. Microbiol. Biotechnol., 2004, 65:649-657
Jiamg M. et al., Oligonucleotides, 2004
Karlsson M C, Guinamard R, Bolland S, Sankala M, Steinman R M, Ravetch J V. Macrophages control the retention and trafficking of B lymphocytes in the splenic marginal zone. J Exp Med. 2003; 198:333-340.
Kashani-Sabet M., Expert Opin. Biol. Ther., 2004, November, 4(11):1749-1755
Kaushansky, K. et al. Nature, 1994, 369:568-571
Kaushansky, K. N Engl J Med., 1998, 339:746-754
Kerr J F, Wyllie A H, Currie A R. Apoptosis: a basic biological phenomenon with wide-ranging implications in tissue kinetics. British Journal of Cancer. 1972; 26:239-257.
Kerr W G, Heller M, Herzenberg L A. Analysis of lipopolysaccharide-response genes in B-lineage cells demonstrates that they can have differentiation stage-restricted expression and contain SH2 domains. Proceedings of the National Academy of Sciences of the United States of America. 1996; 93:3947-3952.
Khrvorova A. et al., Cell, 2003, 115:209-216
Kiel M J, Yilmaz O H, Iwashita T, Yilmaz O H, Terhorst C, Morrison S J. SLAM family receptors distinguish hematopoietic stem and progenitor cells and reveal endothelial niches for stem cells. Cell. 2005; 121:1109-1121.
Kim B. et al., American Journal of Pathology, 2004, 165:2177-2185
Kim, C. H. et al. J Clin Invest., 1999, 104:1751-1759
Kisielow et al., Biochem. J. 363:1-5 (2002)
Kumar, R. et al. Genome Res., 2003, 13:2333-2340
Lambert et al., Drug Deliv. Rev. 47(1): 99-112 (2001)
Lee et al., Nature Biotechnol. 20:500-505 (2002)
Lee S. K. et al., Blood, 2005, 106(3):818-826, epub Apr. 14, 2005
Lewis, D. L. et al. Nat Genet., 2002, 32:107-8
Li, B. et al., Nat. Med., 2005, 11(9), 944-951
Lioubin M N, Algate P A, Tsai S, Carlberg K, Aebersold A, Rohrschneider L R. p 150Ship, a signal transduction molecule with inositol polyphosphate-5-phosphatase activity. Genes Dev. 1996; 10:1084-1095.
Liu et al. Mol Cell Biol., 1994, 14:6926-6935
Liu et al., Genes Dev., 1999, 13(7):789-791
Liu et al.; J. Exp. Med., 1998, 188(7):1333-1342
Liu Y, Jenkins B, Shin J L, Rohrschneider L R. Scaffolding protein Gab2 mediates differentiation signaling downstream of Fms receptor tyrosine kinase. Mol Cell Biol. 2001; 21:3047-3056.
Livingston D. H. et al., Ann. Surg., 2003, 238(5):748-753
Lok, S. et al. Nature, 1994, 369:565-568
Lori, F. et al. Am J Pharmacogenomics, 2002, 2:245-52
Maher, Bioassays 14:807-15 (1992)
Mailand et al., 2002 Nature Cell Biol. 4:317
Manfredsson, F. P. et al. Gene Ther., 2005, epub ahead of print
Matta, H. et al. Cancer Biol Ther., 2003, 2:206-10
Matzke et al., 2001 Science 293:1080
McCaffrey et al., Nature 418(6893):38-39 (2002)
McIntyre G. J. and G. C. Fanning, BMC Biotechnology, 2006, 6:1-8
McManus et al., RNA 8:842-850 (2002)
McManus, M. T. and Sharp, P. A. Nat Rev Genet., 2002, 3:737-47
Meyers and Miller CABIOS, 4:11-17 (1989)
Miller, et al., Biotechniques 7:980-990 (1989)
Minamino et al., Circ. Res. 88:587-592, 2001
Mittal V., Nat. Rev. Genet., 2004, 5(5):355-365
Miyagishi and Taira, Nature Biotechnol. 20:497-500 (2002)
Morris, M. C. et al. Curr Opin Biotechnol., 2000, 11:461-6
Muratovska, A. and Eccles, M. R. FEBS Lett., 2004, 558:63-68
Myers J. W. et al., Nat. Biotechnol., 2003, 21:324-328
Navankasattusas et al., Mol. Cell Biol. 12:1469-1479, 1992
Needleman and Wunsch J. Mol. Biol. 48:444-453 (1970)
Novina, C. D. and Sharp, P. A., Nature, 2004, 430:161-164
Nyknen et al., Cell 107:309-21 (2001)
Oleinikov A. V. et al., Nucleic Acids Research, 2005, 33(10):e92
Paddison et al., Genes Dev. 16:948-958 (2002)
Pai, S. I. et al. Gene Ther., 2005, epub ahead of print
Pancoska P. et al., Nucleic Acids Research, 2004, 32(4):1469-1479
Paraiso K H, Ghansah T, Costello A, Engelman R W, Kerr W G. Induced SHIP deficiency expands myeloid regulatory cells and abrogates graft-versus-host disease. J Immunol. 2007; 178:2893-2900.
Pardridge, W. M. Expert Opin. Biol. Ther., 2004, 4:1103-1113
Paul et al., Nature Biotechnol. 20:505-508 (2002)
Perez, L. E. et al. Exp Hematol., 2004, 32:300-307
Pesesse X, Deleu S, De Smedt F, Drayer L, Erneux C. Identification of a second SH2-domain-containing protein closely related to the phosphatidylinositol polyphosphate 5-phosphatase SHIP. Biochem Biophys Res Commun. 1997; 239:697-700.
Pesesse X, Moreau C, Drayer A L, Woscholski R, Parker P, Emeux C. The SH2 domain containing inositol 5-phosphatase SHTP2 displays phosphatidylinositol 3,4,5-trisphosphate and inositol 1,3,4,5-tetrakisphosphate 5-phosphatase activity. FEBS Letters. 1998; 437:301-303.
Petit I, Szyper-Kravitz M, Nagler A, et al. G-CSF induces stem cell mobilization by decreasing bone marrow SDF-1 and up-regulating CXCR4. Nat Immunol. 2002; 3:687-694.
Plasterk, 2002 Science 296:1263
Provost, P. D. et al. Embo J, 2002, 21:5864
Putnam, Am. J. Health Syst. Pharm. 53(2):151-160 (1996), erratum at Am. J. Health Syst. Pharm. 53(3):325 (1996)
Qin, X. F. et al. Proc Natl Acad Sci USA, 2003, 100:183-8
Raab, R. M. and Stephanopoulos, G. Biotechnol. Bioeng., 2004, 88:121-132
Raoul, C. et al. Gene Ther., 2005, epub ahead of print
Rauh M J, Ho V, Pereira C, et al. SHIP represses the generation of alternatively activated macrophages. Immunity. 2005; 23:361-374.
Reich, S. J. et al. Mol. Vis., 2003, 9:210-216
Reynolds A. et al., Nature Biotechnol., 2004, 22:326-330
Rodriguez-Lebron, E. and Paulson, H. L. Gene Ther., 2005, epub ahead of print
Rohrschneider et al., Genes & Development, 2000, 14:505-520
Ruther et al., Oncogene 8:87-93, 1993
Ryther, R. C. et al. Gene Ther., 2005, 12:5-11
Samara et al., Mol. Cell Biol. 22:4702-4713, 2002
Sarkar T. et al., Nucleic Acids Research, 2005, 33(1):143-151
Sattler M, Verma S, Pride Y B, Salgia R, Rohrschneider L R, Griffin J D. SHIP1, an SH2 domain containing polyinositol-5-phosphatase, regulates migration through two critical tyrosine residues and forms a novel signaling complex with DOK1 and CRKL. J Biol Chem. 2001; 276:2451-2458.
Scadden et al., 2001 EMBO Rep. 2:1107
Scherr, M. et al. (2003a) Cell Cycle, 2:251-7
Scherr, M. et al. (2003b) Curr Med. Chem., 10:245-56
Scherr, M. et al. (2003c) Oligonucleotides, 13:353-363
Schubert S. et al., J. Mol. Biol., 2005, 348:883-893
Schwab et al., Ann. Oncol. 5 Suppl. 4:55-8 (1994)
Schwarz D. S. et al., Cell, 2003, 115:199-208
Semerad C L, Christopher M J, Liu F, et al. G-CSF potently inhibits osteoblast activity and CXCL12 mRNA expression in the bone marrow. Blood. 2005; 106:3020-3027.
Sharp, Genes Dev. 13:139-41 (1999)
Sharp, Genes Dev., 15:485-490 (2001)
Shen, C. et al. FEBS Lett., 2003, 539:111-4
Shen, W. G. Chin. Med. J. (Engl), 2004, 117:1084-1091
Shuey, D. J. et al. Drug Discov Today, 2002, 7:1040-6
Simeoni, F. et al. Nucleic Acids Res., 2003, 31:2717-24
Sinn et al., Physical Genomics 3:25-31, 2000
Sly L M, Rauh M J, Kalesnikoff J, Song C H, Krystal G. LPS-induced upregulation of SHIP is essential for endotoxin tolerance. Immunity. 2004; 21:227-239.
Song E. et al., Nat. Biotechnol., 2005, 23(6):709-717, epub May 22, 2005
Song, E. et al. Nat Med., 2003, 9:347-51
Sorensen, D. R. et al. J Mol Biol., 2003, 327:761-6
Soutschek J. et al., Nature, 2004, 432:173-178
Spagnou, S. et al. Biochemistry, 2004, 43:13348-13356
Sudres M, Norol F, Trenado A, et al. Bone marrow mesenchymal stem cells suppress lymphocyte proliferation in vitro but fail to prevent graft-versus-host disease in mice. J Immunol. 2006; 176:7761-7767.
Sui et al., Proc. Natl. Acad. Sci. USA 99(6):5515-5520 (2002)
Takaku, H. Antivir Chem. Chemother, 2004, 15:57-65
Takeshita S, Namba N, Zhao J J, et al. SHIP-deficient mice are severely osteoporotic due to increased numbers of hyper-resorptive osteoclasts. Nat Med. 2002; 8:943-949.
Tanaka et al., Genomics 58:281-292, 1999
Tridandapani S. et al., J. Immunol., 2002, 169(8):4370-4378
Tu Z, Ninos J M, Ma Z, et al. Embryonic and hematopoietic stem cells express a novel SH2-containing inositol 5′-phosphatase isoform that partners with the Grb2 adapter protein. Blood. 2001; 98:2028-2038.
Tuschl, 2001 Chembiochem. 2:239-245
Tuschl, T., Nature Biotechnol. 20:440-448 (2002)
Ui-Tei K. et al., Nucl. Acids Res., 2004, 32:936-948
Velasco et al., Gene Ther. 8:897-904, 2001
Vibert et al., Eur. J. Biochem. 181:33-39, 1989
Wadhwa, R. et al. Mutat. Res., 2004, 567:71-84
Wahle J A, Paraiso K H, Costello A L, Goll E L, Sentman C L, Kerr W G. Cutting edge: dominance by an MHC-independent inhibitory receptor compromises NK killing of complex targets. J. Immunol. 2006; 176:7165-7169.
Wahle J A, Paraiso K H, Kendig R D, et al. Inappropriate Recruitment and Activity by the Src Homology Region 2 Domain-Containing Phosphatase 1 (SHP1) Is Responsible for Receptor Dominance in the SHIP-Deficient NK Cell. J Immunol. 2007; 179:8009-8015.
Wang J W, Howson J M, Ghansah T, et al. Influence of SHIP on the NK repertoire and allogeneic bone marrow transplantation. Science. 2002; 295:2094-2097.
Wang, J. F. et al. Blood, 1998, 92:756-764
Ware M D, Rosten P, Damen J E, Liu L, Humphries R K, Krystal G. Cloning and characterization of human SHIP, the 145-kD inositol 5-phosphatase that associates with SHC after cytokine stimulation. Blood. 1996; 88:2833-2840.
Wilson et al., 1984 Cell 37:767
Winter, 14(4):239-48
Wright D E, Wagers A J, Gulati A P, Johnson F L, Weissman I L. Physiological migration of hematopoietic stem and progenitor cells. Science. 2001; 294:1933-1936.
Wunsch J. Mol. Biol. 48:444-453 (1970)
Xia et al., Nature Biotechnol. 20(10):1006-10 (2002)
Yang D. et al., Proc. Natl. Acad. Sci. USA, 2002, 99:9942-9947
Yilmaz O H, Valdez R, Theisen B K, et al. Pten dependence distinguishes haematopoietic stem cells from leukaemia-initiating cells. Nature. 2006; 441:475-482.
Yu et al., Proc. Natl. Acad. Sci. USA 99(9):6047-6052 (2002)
Yuan, B. et al. “siRNA Selection Server: an automated siRNA oligonucleotide prediction server”, Nucleic Acids Research, 2004, Vol. 32, W130-W134, Web Server issue
Zamore 2001 Nat. Struct. Biol. 8:746
Zamore et al., Cell 101:25-33 (2000)
Zamore, 2002 Science 296:1265
Zeng et al., Mol. Cell 9:1327-1333 (2002)
Zhang et al., Gene 273:239-249, 2001
Zhang J, Grindley J C, Yin T, et al. PTEN maintains haematopoietic stem cells and acts in lineage choice and leukaemia prevention. Nature. 2006; 441:518-522.
Zhang J, Niu C, Ye L, et al. Identification of the haematopoietic stem cell niche and control of the niche size. Nature. 2003; 425:836-841.
Zhang, X. et al. J. Biol. Chem., 2004, 279:10677-10684
Zheng, B. J. Antivir. Ther., 2004, 9:365-374
Zhu et al., Mol. Cell Biol. 13:4432-4444, 1993
Zimmermann T. S. et al., Nature, 2006, 441:111-114
Zippo A, De Robertis A, Bardelli M, Galvagni F, Oliviero S. Identification of Flk-1 target genes in vasculogenesis: Pim-1 is required for endothelial and mural cell differentiation in vitro. Blood. 2004; 103:4536-4544.

1. A method for directing hematopoietic stem cells (HSC) from bone marrow to another site in the body of a person or animal, wherein said method comprises inhibiting SHIP expression and/or SHIP function or activity in a cell of the person or animal, such that the HSC are directed to another site in the body.

2. The method according to claim 1, wherein SHIP expression is inhibited by RNA interference, or antisense inhibition, or ribozyme inactivation, or an antibody or other molecule that inhibits function or activity of SHIP.

3. (canceled)

4. The method according to claim 1, wherein the person or animal has radiation injury manifesting as impaired bone marrow function, or was exposed to, or is at risk of exposure to, a radioisotope that can accumulate in bone.

5. (canceled)

6. The method according to claim 1, wherein the person or animal has, or is at risk of developing, fibrotic and/or damaged bone marrow due to chemotherapy and/or radiation therapy.

7. The method according to claim 1, wherein the person or animal is being treated with one or more other treatments to increase hematopoietic function.

8. (canceled)

9. The method according to claim 1, wherein the HSC are directed from bone marrow to the spleen and/or liver.

10. (canceled)

11. The method according to claim 1, wherein the person or animal has an infection or a genetic or congenital defect that impairs bone marrow function.

12-13. (canceled)

14. The method according to claim 1, wherein the person or animal has bone marrow impairment that results from Fanconi anemia, dyskeratosis congenital, Diamond-Blackfan anemia, Shwachman-Diamond syndrome, aplastic anemia, MDS, PRCA, or myelophthisis.

15-16. (canceled)

17. A method for rescuing or enhancing blood cell recovery during bone marrow impairment in a person or animal, wherein said method comprises inhibiting SHIP expression and/or SHIP function or activity in a cell of the person or animal.

18-19. (canceled)

20. The method according to claim 17, wherein the bone marrow impairment results from Fanconi anemia, dyskeratosis congenital, Diamond-Blackfan anemia, Shwachman-Diamond syndrome, aplastic anemia, MDS, PRCA, or myelophthisis.

21. The method according to claim 17, wherein SHIP expression is inhibited by RNA interference, or antisense inhibition, or ribozyme inactivation, or an antibody or other molecule that inhibits function or activity of SHIP.

22. (canceled)

23. The method according to claim 17, wherein the person or animal has radiation injury manifesting as impaired bone marrow function, or was exposed to, or is at risk of exposure to, a radioisotope that can accumulate in bone.

24. (canceled)

25. The method according to claim 17, wherein the person or animal has, or is at risk of developing, fibrotic and/or damaged bone marrow due to chemotherapy and/or radiation therapy.

26. The method according to claim 17, wherein the person or animal is being treated with one or more other treatments to increase hematopoietic function.

27. (canceled)

28. The method according to claim 17, wherein HSC are directed from bone marrow to the spleen and/or liver.

29. (canceled)

30. The method according to claim 17, wherein the person or animal has an infection or a genetic or congenital defect that impairs bone marrow function.

31-32. (canceled)

33. A method for treating or delaying the onset of impaired bone marrow function in a person or animal, wherein said method comprises administering a therapeutically effective amount of a SHIP inhibitor to a person or animal in need of treatment.

34. The method according to claim 33, wherein SHIP expression is inhibited by RNA interference, or antisense inhibition, or ribozyme inactivation, or an antibody or other molecule that inhibits function or activity of SHIP.

35. (canceled)

36. The method according to claim 33, wherein the person or animal has radiation injury manifesting as impaired bone marrow function, or was exposed to, or is at risk of exposure to, a radioisotope that can accumulate in bone.

37. (canceled)

38. The method according to claim 33, wherein the person or animal has, or is at risk of developing, fibrotic and/or damaged bone marrow due to chemotherapy and/or radiation therapy.

39. The method according to claim 33, wherein the person or animal is being treated with one or more other treatments to increase hematopoietic function.

40. (canceled)

41. The method according to claim 33, wherein the HSC are directed from bone marrow to the spleen and/or liver.

42. (canceled)

43. The method according to claim 33, wherein the person or animal has an infection or a genetic or congenital defect that impairs bone marrow function.

44-45. (canceled)

46. The method according to claim 33, wherein the person or animal has bone marrow impairment that results from Fanconi anemia, dyskeratosis congenital, Diamond-Blackfan anemia, Shwachman-Diamond syndrome, aplastic anemia, MDS, PRCA, or myelophthisis.

47-48. (canceled)


SEQUENCE LISTING (23 sequences)

SEQ ID NO: 1 - DNA, 4925 bp, Homo sapiens
aaacaggaagtcagtcagttaagctggtggcagcagccgaggccaccaagaggcaacgggcggcaggttgcagtggaggggcctccgctcccctcggtggtgtgtgggtcctgggggtgcctgccggcccggccgaggaggcccacgcccaccatggtcccctgctggaaccatggcaacatcacccgctccaaggcggaggagctgctttccaggacaggcaaggacgggagcttcctcgtgcgtgccagcgagtccatctcccgggcatacgcgctctgcgtgctgtatcggaattgcgtttacacttacagaattctgcccaatgaagatgataaattcactgttcaggcatccgaaggcgtctccatgaggttcttcaccaagctggaccagctcatcgagttttacaagaaggaaaacatggggctggtgacccatctgcaataccctgtgccgctggaggaagaggacacaggcgacgaccctgaggaggacacagaaagtgtcgtgtctccacccgagctgcccccaagaaacatcccgctgactgccagctcctgtgaggccaaggaggttcctttttcaaacgagaatccccgagcgaccgagaccagccggccgagcctctccgagacattgttccagcgactgcaaagcatggacaccagtgggcttccagaagagcatcttaaggccatccaagattatttaagcactcagctcgcccaggactctgaatttgtgaagacagggtccagcagtcttcctcacctgaagaaactgaccacactgctctgcaaggagctctatggagaagtcatccggaccctcccatccctggagtctctgcagaggttatttgaccagcagctctccccgggcctccgtccacgtcctcaggttcctggtgaggccaatcccatcaacatggtgtccaagctcagccaactgacaagcctgttgtcatccattgaagacaaggtcaaggccttgctgcacgagggtcctgagtctccgcaccggccctcccttatccctccagtcacctttgaggtgaaggcagagtctctggggattcctcagaaaatgcagctcaaagtcgacgttgagtctgggaaactgatcattaagaagtccaaggatggttctgaggacaagttctacagccacaagaaaatcctgcagctcattaagtcacagaaatttctgaataagttggtgatcttggtggaaacagagaaggagaagatcctgcggaaggaatatgtttttgctgactccaaaaagagagaaggcttctgccagctcctgcagcagatgaagaacaagcactcagagcagccggagcccgacatgatcaccatcttcatcggcacctggaacatgggtaacgccccccctcccaagaagatcacgtcctggtttctctccaaggggcagggaaagacgcgggacgactctgcggactacatcccccatgacatttacgtgatcggcacccaagaggaccccctgagtgagaaggagtggctggagatcctcaaacactccctgcaagaaatcaccagtgtgacttttaaaacagtcgccatccacacgctctggaacatccgcatcgtggtgctggccaagcctgagcacgagaaccggatcagccacatctgtactgacaacgtgaagacaggcattgcaaacacactggggaacaagggagccgtgggggtgtcgttcatgttcaatggaacctccttagggttcgtcaacagccacttgacttcaggaagtgaaaagaaactcaggcgaaaccaaaactatatgaacattctccggttcctggccctgggcgacaagaagctgagtccctttaacatcactcaccgcttcacgcacctcttctggtttggggatcttaactaccgtgtggatctgcctacctgggaggcagaaaccatcatccagaaaatcaagcagcagcagtacgcagacctcctgtcccacgaccagctgctcacagagaggagggagcagaaggtcttcctacacttcgaggaggaagaaatcacgtttgccccaacctaccgttttgagagactgactcgggacaaatacgcctacaccaagcagaaagcgacagggatgaagtacaacttgccttcctggtgtgaccgagtcctctggaagtcttatcccctggtgcacgtggtgtgtcagtcttatggcagtaccagcgacatcatgacgagtgaccacagccctgtctttgccacatttgaggcaggagtcacttcccagtttgtctccaagaacggtcccgggactgttgacagccaaggacagattgagtttctcaggtgctatgccacattgaagaccaagtcccagaccaaattctacctggagttccactcgagctgcttggagagttttgtcaagagtcaggaaggagaaaatgaagaaggaagtgagggggagctggtggtgaagtttggtgagactcttccaaagctgaagcccattatctctgaccctgagtacctgctagaccagcacatcctcatcagcatcaagtcctctgacagcgacgaatcctatggcgagggctgcattgcccttcggttagaggccacagaaacgcagctgcccatctacacgcctctcacccaccatggggagttgacaggccacttccagggggagatcaagctgcagacctctcagggcaagacgagggagaagctctatgactttgtgaagacggagcgtgatgaatccagtgggccaaagaccctgaagagcctcaccagccacgaccccatgaagcagtgggaagtcactagcagggcccctccgtgcagtggctccagcatcactgaaatcatcaaccccaactacatgggagtggggccctttgggccaccaatgcccctgcacgtgaagcagaccttgtcccctgaccagcagcccacagcctggagctacgaccagccgcccaaggactccccgctggggccctgcaggggagaaagtcctccgacacctcccggccagccgcccatatcacccaagaagtttttaccctcaacagcaaaccggggtctccctcccaggacacaggagtcaaggcccagtgacctggggaagaacgcaggggacacgctgcctcaggaggacctgccgctgacgaagcccgagatgtttgagaaccccctgtatgggtccctgagttccttccctaagcctgctcccaggaaggaccaggaatcccccaaaatgccgcggaaggaacccccgccctgcccggaacccggcatcttgtcgcccagcatcgtgctcaccaaagcccaggaggctgatcgcggcgaggggcccggcaagcaggtgcccgcgccccggctgcgctccttcacgtgctcatcctctgccgagggcagggcggccggcggggacaagagccaagggaagcccaagaccccggtcagctcccaggccccggtgccggccaagaggcccatcaagccttccagatcggaaatcaaccagcagaccccgcccaccccgacgccgcggccgccgctgccagtcaagagcccggcggtgctgcacctccagcactccaagggccgcgactaccgcgacaacaccgagctcccgcatcacggcaagcaccggccggaggaggggccaccagggcctctaggcaggactgccatgcagtgaagccctcagtgagctgccactgagtcgggagcccagaggaacggcgtgaagccactggaccctctcccgggacctcctgctggctcctcctgcccagcttcctatgcaaggctttgtgttttcaggaaagggcctagcttctgtgtggcccacagagttcactgcctgtgagacttagcaccaagtgctgaggctggaagaaaaacgcacaccagacgggcaacaaacagtctgggtccccagctcgctcttggtacttgggaccccagtgcctcgttgagggcgccattctgaagaaaggaactgcagcgccgatttgagggtggagatatagataataataatattaataataataatggccacatggatcgaacactcatgatgtgccaagtgctgtgctaagtgctttacgaacattcgtcatatcaggatgacctcgagagctgaggctctagccacctaaaaccacgtgcccaaacccaccagtttaaaacggtgtgtgttcggaggggtgaaagcattaagaagcccagtgccctcctggagtgagacaagggctcggccttaaggagctgaagagtctgggtagcttgtttagggtacaagaagcctgttctgtccagcttcagtgacacaagctgctttagctaaagtcccgcgggttccggcatggctaggctgagagcagggatctacctggcttctcagttctttggttggaaggagcaggaaatcagctcctattctccagtggagagatctggcctcagcttgggctagagatgccaaggcctgtgccaggttccctgtgccctcctcgaggtgggcagccatcaccagccacagttaagccaagccccccaacatgtattccatcgtgctggtagaagagtctttgctgttgctcccgaaagccgtgctctccagcctggctgccagggagggtgggcctcttggttccaggctcttgaaatagtgcagccttttcttcctatctctgtggctttcagctctgcttccttggttattaggagaatagatgggtgatgtctttccttatgttgctttttcaacatagcagaattaatgtagggagctaaatccagtggtgtgtgtgaatgcagaagggaatgcaccccacattcccatgatggaagtctgcgtaaccaataaattgtgcctttctcactcaaaaaaaaaaa

SEQ ID NO: 2 - DNA, 4938 bp, Mus musculus
agtttcctctccctcgctgagtgcctgaaacaggaagtcagtcagttaagctggtaggagcagcagaggcaatttctgagaggcaacaggcggcaggtctcagcctagagagggccctgaactactttgctggagtgtccgtcctgggagtggctgctgacccagtccaggagacccatgcctgccatggtccctgggtggaaccatggcaacatcacccgctccaaggcagaggagctactttccagagccggcaaggacgggagcttccttgtgcgtgccagcgagtccatcccccgggcctacgcactctgcgtgctgttccggaattgtgtttacacttacaggattctgcccaatgaggacgataaattcactgttcaggcatccgaaggtgtccccatgaggttcttcacgaagctggaccagctcatcgacttttacaagaaggaaaacatggggctggtgacccacctgcagtaccccgtgcccctggaggaggaggatgctattgatgaggctgaggaggacactgtagaaagtgtcatgtcaccacctgagctgcctcccagaaacattcctatgtctgccgggcccagcgaggccaaggaccttcctcttgcaacagagaacccccgagcccctgaggtcacccggctgagtctctccgagacactgtttcagcgtctacagagcatggataccagtgggcttcccgaggagcacctgaaagccatccaggattatctgagcactcagctcctcctggattccgactttttgaagacgggctccagcaacctccctcacctgaagaagctgatgtcactgctctgcaaggagctccatggggaagtcatcaggactctgccatccctggagtctctgcagaggttgtttgaccaacagctctccccaggccttcgcccacgacctcaggtgcccggagaggccagtcccatcaccatggttgccaaactcagccaattgacaagtctgctgtcttccattgaagataaggtcaagtccttgctgcacgagggctcagaatctaccaacaggcgttcccttatccctccggtcacctttgaggtgaagtcagagtccctgggcattcctcagaaaatgcatctcaaagtggacgttgagtctgggaaactgatcgttaagaagtccaaggatggttctgaggacaagttctacagccacaaaaaaatcctgcagctcattaagtcccagaagtttctaaacaagttggtgattttggtggagacggagaaggagaaaatcctgaggaaggaatatgtttttgctgactctaagaaaagagaaggcttctgtcaactcctgcagcagatgaagaacaagcattcggagcagccagagcctgacatgatcaccatcttcattggcacttggaacatgggtaatgcaccccctcccaagaagatcacgtcctggtttctctccaaggggcagggaaagacacgggacgactctgctgactacatcccccatgacatctatgtgattggcacccaggaggatccccttggagagaaggagtggctggagctactcaggcactccctgcaagaagtcaccagcatgacatttaaaacagttgccatccacaccctctggaacattcgcatagtggtgcttgccaagccagagcatgagaatcggatcagccatatctgcactgacaacgtgaagacaggcatcgccaacaccctgggaaacaagggagcagtgggagtgtccttcatgttcaatggaacctccttggggttcgtcaacagccacttgacttctggaagtgaaaaaaagctcaggagaaatcaaaactatatgaacatcctgcggttcctggccctgggagacaagaagctaagcccatttaacatcacccaccgcttcacccacctcttctggcttggggatctcaactaccgcgtggagctgcccacttgggaggcagaggccatcatccagaagatcaagcaacagcagtattcagaccttctggcccacgaccaactgctcctggagaggaaggaccagaaggtcttcctgcactttgaggaggaagagatcaccttcgcccccacctatcgatttgaaagactgacccgggacaagtatgcatacacgaagcagaaagcaacagggatgaagtacaacttgccgtcctggtgcgaccgagtcctctggaagtcttacccgctggtgcatgtggtctgtcagtcctatggcagtaccagtgacatcatgacgagtgaccacagccctgtctttgccacgtttgaagcaggagtcacatctcaattcgtctccaagaatggtcctggcactgtagatagccaagggcagatcgagtttcttgcatgctacgccacactgaagaccaagtcccagactaagttctacttggagttccactcaagctgcttagagagttttgtcaagagtcaggaaggagagaatgaagagggaagtgaaggagagctggtggtacggtttggagagactcttcccaagctaaagcccattatctctgaccccgagtacttactggaccagcatatcctgatcagcattaaatcctctgacagtgacgagtcctatggtgaaggctgcattgcccttcgcttggagaccacagaggctcagcatcctatctacacgcctctcacccaccatggggagatgactggccacttcaggggagagattaagctgcagacctcccagggcaagatgagggagaagctctatgactttgtgaagacagagcgggatgaatccagtggaatgaaatgcttgaagaacctcaccagccatgaccctatgaggcaatgggagccttctggcagggtccctgcatgtggtgtctccagcctcaatgagatgatcaatccaaactacattggtatggggccttttggacagcccctgcatgggaaatcaaccctgtccccagatcagcaactcacagcttggagttatgaccagctacccaaagactcctccctggggcctgggaggggggagggtcctccaacccctccctcccaaccacctctgtcgccaaagaagttttcatcttccacagccaaccgaggtccctgccccagggtgcaagaggcaagacctggggatctgggaaaggtggaagctctgctccaggaggacctgctgctgacgaagcccgagatgtttgagaacccactgtatggatccgtgagttccttccctaagctggtgcccaggaaagagcaggagtctcccaagatgctgcggaaggagcccccgccctgtccagacccaggaatctcatcacccagcatcgtgctccccaaagcccaagaggtggagagtgtcaaggggacaagcaaacaggcccctgtgcctgtccttggccccacaccccggatccgctcctttacctgttcttcttctgctgagggcagaatgaccagtggggacaagagccaagggaagcccaaggcctcagccagttcccaagccccagtgccagtcaagaggcctgtcaagccttccaggtcagaaatgagccagcagacaacacccatcccagctccacggccacccctgccagtcaagagtcctgctgtcctgcagctgcaacattccaaaggcagagactaccgtgacaacacagaactcccccaccatggcaagcaccgccaagaggaggggctgcttggcaggactgccatgcagtgagctgctggtgatcggagcctggaggaacagcacaaagcagacctgcgcctctctcaggatgcctctctcaggatgcctcttggaggacctcctgctagctcttcttgcctagcttcaagtcccaggctgtgtattttttttcaggaaacggcctcacttctctgtggtccaagaagtgtgctgctggctgccacactgtgcggcagatgctaaagctggatgacaaacgcacgccatacagacagcagacagcggcactgggtctcagaacttggattcctgggccttcttccagtcgccgttttaaagaaaggaactaacggagctgctcatccgagggtgaagatataaataataatattattaataataataacagtcaggtgccatgtgctgtgttaagtgctttatgaacatttgtcgggctggcctccagtgctgaggtgccagtcagcctgaaccctatgcccaggcccactaatcccaaatggtgggtcctgagatgtttttaaaaagcattaaagaaaaccatcggtctcttagagctaaccggccgggctctactgcagggacccgaacagtctgcatggctaagtggcacaaggagcctggccctgtccagcttcagagatccaagctgctttttgctggggttctgtcacaggcctgatcctcttggtttttatggggtttcaagtctgccagagtcagaaatcagctctaactcgccagtgaagagatctggccttaacttaagccagccacgtcaggcccctgctgagcctatggaccaataaatactccccgtgccactggaggtgggcagctatcaccataccctgagttgggccaagcccaccccacccctaccctgcaacatttctgatgtactgaggaagagtctccaccatagtccccaagggctgagttctccagcctgctatcagggaaggtgagcattggtcccaggctctcaaaatagtgcagcctcttcttcccaagctctggggtgcaccctgtgtccttggttaccaggagactagggttgtgatatcttttcttgtcttgctttttgatatatcaggattaatgtaggaaaccagacctagattattcaggagagtaggtatatcccctgtgtttcccagtctgagtgaccaataaaattgtgcctttctaactggg

SEQ ID NO: 3 - DNA, 19 bp, Homo sapiens
gcctgttgtcatccattga

SEQ ID NO: 4 - DNA, 19 bp, Homo sapiens
ataagttggtgatcttggt

SEQ ID NO: 5 - DNA, 19 bp, Homo sapiens
gccacatctgtactgacaa

SEQ ID NO: 6 - DNA, 19 bp, Homo sapiens
agacaggcattgcaaacac

SEQ ID NO: 7 - DNA, 19 bp, Homo sapiens
acatcactcaccgcttcac

SEQ ID NO: 8 - DNA, 19 bp, Homo sapiens
tcttaactaccgtgtggat

SEQ ID NO: 9 - DNA, 19 bp, Homo sapiens
aatacgcctacaccaagca

SEQ ID NO: 10 - DNA, 19 bp, Homo sapiens
gtaccagcgacatcatgac

SEQ ID NO: 11 - DNA, 19 bp, Homo sapiens
gcgacatcatgacgagtga

SEQ ID NO: 12 - DNA, 19 bp, Homo sapiens
aggacagattgagtttctc

SEQ ID NO: 13 - DNA, 19 bp, Homo sapiens
ggtgctatgccacattgaa

SEQ ID NO: 14 - DNA, 19 bp, Homo sapiens
gtttggtgagactcttcca

SEQ ID NO: 15 - DNA, 19 bp, Homo sapiens
agacggagcgtgatgaatc

SEQ ID NO: 16 - RNA, 21 nt, Homo sapiens
aaggaauugcguuuacacuua

SEQ ID NO: 17 - RNA, 21 nt, Homo sapiens
aaaauugcguuuacacuuaca

SEQ ID NO: 18 - DNA, 97 bp, Homo sapiens
tgctgttgacagtgagcgaggcttccagaagagcatcttatagtgaagccacagatgtataagatgctcttctggaagccctgcctactgcctcgga

SEQ ID NO: 19 - DNA, 97 bp, Homo sapiens
tgctgttgacagtgagcgagcccatatcacccaagaagtttagtgaagccacagatgtaaacttcttgggtgatatgggcgtgcctactgcctcgga

SEQ ID NO: 20 - DNA, 21 bp, Homo sapiens
gcttccagaagagcatcttat

SEQ ID NO: 21 - DNA, 22 bp, Homo sapiens
gcccatatcacccaagaagttt

SEQ ID NO: 22 - DNA, 21 bp, Homo sapiens
aaggaattgcgtttacactta

SEQ ID NO: 23 - DNA, 21 bp, Homo sapiens
aaaattgcgtttacacttaca